(19) United States
(12) Patent Application Publication
(10) Pub. No.: US 2011/0195505 A1
(43) Pub. Date: Aug. 11, 2011

(54) BACTERIAL STRAINS FOR BUTANOL PRODUCTION

(75) Inventors: LORI JEAN EULER, WOODBURY, NJ (US); DENNIS FLINT, NEWARK, DE (US); BRIAN JAMES PAUL, WILMINGTON, DE (US); TINA K. VAN DYK, WILMINGTON, DE (US); RICK W. YE, HOCKESSIN, DE (US)

(73) Assignee: BUTAMAX(TM) ADVANCED BIOFUELS LLC, WILMINGTON, DE (US)

(21) Appl. No.: 12/899,760

(22) Filed: Oct. 7, 2010

Related U.S. Application Data
(60) Provisional application No. 61/249,792, filed on Oct. 8, 2009.

(51) Int. Cl. C12N 15/74; C12N 1/21; C12N 1/36

(52) U.S. Cl. 435/471; 435/252.3; 435/252.31; 435/252.32; 435/252.33; 435/252.34; 435/245

(57) ABSTRACT

Bacteria that are not natural butanol producers were found to have increased tolerance to butanol when the saturated fatty acids content in bacterial cell membrane was increased. Methods for increasing the concentration of saturated fatty acids in the membranes of bacteria that are not natural butanol produces are described whereby tolerance of the bacterial cell to butanol is increased. Saturated fatty acids concentration in the bacterial cell membrane increased upon exogenously feeding saturated fatty acids to cells. Bacterial strains useful for production of butanol are described herein having modified unsaturated fatty acid biosynthetic pathway.

### CROSS-REFERENCE TO RELATED APPLICATIONS

This application claims the benefit of priority of U.S. Provisional Application No. 61/249,792, filed on Oct. 8, 2009, the entirety of which is herein incorporated by reference.

### FIELD OF INVENTION

The invention relates to the fields of microbiology and genetic engineering. More specifically altered saturated fatty acid composition was found to play a role in butanol tolerance of bacteria.

### BACKGROUND OF INVENTION

Butanol is an important industrial chemical, useful as fuel additive and feedstock chemical in the plastics industry and as a food grade extractant in the food and flavor industry. About 10 to 12 billion pounds of butanol are produced annually by petrochemical routes. With the market trends shifting away from fossil fuel dependence and the increasing feasibility of butanol production by non-petrochemical routes, growth in future demand for butanol is highly likely.

Acetone-butanol-ethanol (ABE) fermentation by Clostridium acetobutylicum is one of the oldest known industrial fermentations, and the pathways and genes responsible for the production of these solvents have been reported (Girbal et al., Trends in Biotechnology 16:11-16 (1998)). Recombinant microbial production hosts, expressing a 1-butanol biosynthetic pathway (Donaldson et al., U.S. Patent Application Publication No. US20080182308A1), a 2-butanol biosynthetic pathway (Donaldson et al., U.S. Patent Publication Nos. US 20070259410A1 and US 20070292927), and an isobutanol biosynthetic pathway (Maggio-Hall et al., U.S. Patent Publication No. US 20070092957) have been described. Bacteria of the genus Clostridium naturally produce butanol and have some natural tolerance to butanol. Strains of Clostridium that have increased tolerance to 1-butanol have been isolated by chemical mutagenesis (Jain et al. U.S. Pat. No. 5,192,673; and Blaschek et al. U.S. Pat. No. 6,358,717), over-expression of certain stress response genes (Papoutsakis et al. U.S. Pat. No. 6,960,465; and Tomas et al., Appl. Environ. Microbiol. 69(8):4951-4965 (2003)), and by serial enrichment (Quratulain et al., Folia Microbiologica (Prague) 40(5):467-471 (1995); and Soucaille et al., Current Microbiology 14(5):295-299 (1987)). Overexpression in Clostridium of the endogenous gene encoding cyclopropane fatty acid synthase increased the cyclopropane fatty acid content of early log phase cells and initial butanol resistance (Zhao et al. (2003) Appl. and Environ. Microbiology 69:2831-2841).

In United States Patent Application Publication No. 20090203097, screening of fatty acid fed bacteria which are not natural butanol producers identified increased membrane cyclopropane fatty acid as providing improved butanol tolerance. Increasing expression of cyclopropane fatty acid synthase in the presence of the enzyme substrate that is either endogenous to the cell or fed to the cell, increased butanol tolerance. Bacterial strains with increased cyclopropane fatty acid synthase and having a butanol biosynthetic pathway were found to be useful for production of butanol. In general, bacteria and yeast that are not natural producers of butanol are sensitive to butanol in the medium. A need remains therefore, for bacterial host strains which do not naturally produce butanol and can be engineered to express a butanol biosynthetic pathway, to be more tolerant to these chemicals. A need also remains to further improve butanol tolerance of natural butanol producers. In addition there is a need for methods of producing butanol using bacterial host strains engineered for butanol production that are more tolerant to these chemicals.

### SUMMARY OF THE INVENTION

This invention provides a method for increasing the tolerance of a bacterial cell to butanol comprising increasing the concentration of saturated fatty acids in the membrane of the bacterial cell whereby the tolerance of the bacterial cell to butanol is increased as compared with a bacterial cell where the concentration of saturated fatty acids in the membrane has not been increased.

Accordingly, a lactobacillus cell is described having a genetic modification comprising one or more genes selected from the group consisting of fabA, fabM, fabN, fabZ and fabZ1 and having at least about a 10% increase in total cell membrane saturated fatty acids as compared with a wild-type lactobacillus cell.

Also described is a lactobacillus cell comprising:

(i) altered activity for isomerization of β-hydroxyacyl-ACP dehydratase activity and trans-2-decenoyl-ACP to cis-3-decenoyl-ACP isomerization activity; and

(ii) at least 10% increase in total cell membrane saturated fatty acids as compared with a wild-type lactobacillus cell.

Additionally, a bacterial cell is described for the production of butanol comprising:
a) a butanol biosynthetic pathway,
b) a cell membrane having at least about a 10% increase in total cell membrane saturated fatty acid content as compared with a parent bacterial cell;

wherein the butanol biosynthetic pathway comprises at least one gene that is heterologous to the bacterial cell.

The invention further describes a method of increasing the tolerance of a bacterial cell to butanol comprising altering molar ratios of saturated/unsaturated fatty acid composition in the membrane of the bacterial cell by feeding at least one saturated fatty acid.

A method of altering molar ratios of saturated/unsaturated fatty acid composition in the membrane of a bacterial cell by feeding at least one saturated fatty acid is also described.

Additionally, a Lactobacillus plantarum mutant is described lacking activity for isomerization of trans-2-decenoyl-Acyl Carrier Protein to cis-3-decenoyl-Acyl Carrier Protein.

### BRIEF DESCRIPTION OF THE DRAWINGS AND SEQUENCE DESCRIPTIONS

The various embodiments of the invention can be more fully understood from the following detailed description, the figures, and the accompanying sequence descriptions, which form a part of this application.

FIG. 1 shows a graph of the growth rate of BP63 (ΔfabZ1) at various concentrations of isobutanol and oleic acid.

The following sequences conform with 37 C.F.R. 1.821 1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a bis), and Section 208 and Annex C of the Administrative Instructions). The symbols and format used for nucleotide and amino acid sequence data comply with the rules set forth in 37 C.F.R. §1.822.

TABLE 1
Summary of Representative Gene and Protein SEQ ID Numbers
for 1-Butanol Biosynthetic Pathway

| | SEQ ID | |
|---|---|---|
| | NO: | SEQ ID |
| | Nucleic | NO: |
| Description | acid | Peptide |
| Acetyl-CoA acetyltransferase thlA from | 1 | 2 |
| Clostridium acetobutylicum ATCC 824 | | |
| Acetyl-CoA acetyltransferase thlB from | 3 | 4 |
| Clostridium acetobutylicum ATCC 824 | | |
| 3-Hydroxybutyryl-CoA dehydrogenase | 5 | 6 |
| from Clostridium acetobutylicum ATCC | | |
| 824 | | |
| Crotonase from Clostridium | 7 | 8 |
| acetobutylicum ATCC 824 | | |
| Putative trans-enoyl CoA reductase from | 9 | 10 |
| Clostridium acetobutylicum ATCC 824 | | |
| Euglena gracilis butyryl-CoA | 39 | 40 |
| dehydrogenase/trans-2-enoyl-CoA | | |
| reductase codon optimized | | |
| Butyraldehyde dehydrogenase from | 11 | 12 |
| Clostridium beijerinckii NRRL B594 | | |
| 1-Butanol dehydrogenase bdhB from | 13 | 14 |
| Clostridium acetobutylicum ATCC 824 | | |
| 1-Butanol dehydrogenase | 15 | 16 |
| bdhA from Clostridium acetobutylicum | | |
| ATCC 824 | | |

TABLE 2
Summary of Representative Gene and Protein SEQ ID Numbers
for 2-Butanol Biosynthetic Pathway

| | SEQ ID | SEQ ID |
|---|---|---|
| | NO: | NO: |
| Description | Nucleic acid | Peptide |
| budA, acetolactate decarboxylase from | 17 | 18 |
| Klebsiella pneumoniae ATCC 25955 | | |
| budB, acetolactate synthase from Klebsiella | 19 | 20 |
| pneumoniae ATCC 25955 | | |
| budC, butanediol dehydrogenase from | 21 | 22 |
| Klebsiella pneumoniae IAM1063 | | |
| pddA, butanediol dehydratase alpha subun [symbol] | 23 | 24 |
| from Klebsiella oxytoca ATCC 8724 | | |
| pddB, butanediol dehydratase beta subunit | 25 | 26 |
| from Klebsiella oxytoca ATCC 8724 | | |
| pddC, butanediol dehydratase gamma | 27 | 28 |
| subunit from Klebsiella oxytoca ATCC 8724 | | |
| sadH, 2-butanol dehydrogenase from | 29 | 30 |
| Rhodococcus ruber 219 | | |

[symbol]

indicates data missing or illegible when filed

TABLE 3
Summary of Representative Gene and Protein SEQ ID Numbers
for Isobutanol Biosynthetic Pathway

| | | SEQ ID |
|---|---|---|
| | SEQ ID NO: | NO: |
| Description | Nucleic acid | Peptide |
| Klebsiella pneumoniae budB | 19 | 20 |
| (acetolactate synthase) | | |
| Escherichia coli ilvC (acetohydroxy acid | 31 | 32 |
| reductoisomerase) | | |
| B. subtilis ilvC (acetohydroxy acid | 41 | 42 |
| reductoisomerase) | | |
| Escherichia coli ilvD (acetohydroxy acid | 33 | 34 |
| dehydratase) | | |
| Lactococcus lactis kivD (branched-chain | 35 | 36 |
| α-keto acid decarboxylase), codon | | |
| optimized | | |
| Escherichia coli yqhD (branched-chain | 37 | 38 |
| alcohol dehydrogenase) | | |

TABLE 4
Representative Nucleic Acid and Amino Acid Sequences for an
enzyme comprising activity for isomerization of trans-2-decenoyl-Acyl
Carrier Protein to cis-3-decenoyl-Acyl Carrier Protein.

| | SEQ ID | SEQ ID |
|---|---|---|
| | NO: | NO: |
| Description | Nucleic acid | Peptide |
| Lactobacillus plantarum strain WCFS1, | 107 | 94 |
| fabZ1 | | |
| Lactobacillus sakei subsp. sakei 23K, | 108 | 95 |
| fabZ1 | | |
| Lactobacillus plantarum strain JDM1, | 109 | 96 |
| fabZ1 | | |
| Lactococcus lactis subsp. lactis IL1403, | 110 | 97 |
| fabZ1 | | |
| Leuconostoc citreum KM20, fabZ1 | 111 | 98 |
| Lactobacillus plantarum subsp. plantarum | 112 | 99 |
| ATCC 14917, fabZ1 | | |
| Lactobacillus ultunensis DSM 16047, | 113 | 100 |
| fabZ1 | | |
| Lactobacillus delbrueckii subsp. | 114 | 101 |
| bulgaricus ATCC 11842, fabZ1 | | |
| Enterococcus faecalis V583, fabZ1, fabN | 115 | 102 |
| Lactobacillus brevis ATCC 367, fabZ | 116 | 103 |
| Pediococcus pentosaceus ATCC 25745, | 117 | 104 |
| fabZ | | |
| Lactobacillus helveticus DPC 4571, fabZ | 118 | 105 |
| Lactobacillus salivarius UCC118, fabZ | 119 | 106 |
| Escherichia coli BL21, fabA | 121 | 120 |
| Lactobacillus reuteri ATCC 55730, fabA | 123 | 122 |
| (also fabZ) | | |
| Agrobacterium radiobacter K84, fabA | 125 | 124 |
| Streptococcus pneumoniae UA159, fabM | 127 | 126 |
| Lactobacillus plantarum strain PN0512, | 129 | 128 |
| fabZ1 | | |

SEQ ID NOs: 43-46 are primers for amplifying a fusion construct containing genes flanking pyrF, with pyrF deleted.

SEQ ID NOs: 47-51 are primers for identifying and sequencing clones containing pyrF deletion on the integration vector.

SEQ ID NOs: 52-57 are primers for differentiating ΔpyrF double cross over recombinants from the background.

SEQ ID NOs: 58 and 59 are primers for amplifyng pyrF from L. plantarum strain PN0512.

SEQ ID NOs: 60 and 61 are primers for amplifying erm promoter.

SEQ ID NOs: 62 and 63 are primers for amplifying fabZ1 upstream homologous arm.

SEQ ID NOs: 64 and 65 are primers for amplifying fabZ1 downstream homologous arm.

SEQ ID NOs: 66 and 67 are primers for differentiating ΔfabZ1 single cross over recombinants from the background.

SEQ ID NOs: 67 and 68 are primers for differentiating ΔfabZ1 double cross over recombinants from the background.

SEQ ID NOs: 69 and 70 are primers for amplification of fabZ1 gene from L. plantarum strain PN0512.

SEQ ID NOs: 70 and 71 are primers for screening clones expressing fabZ1 gene under the control of clpL promoter.

SEQ ID NO 72 is nucleic acid sequence encoding pFP996 PclpL.

SEQ ID NO 73 is nucleic acid sequence encoding pFP996 PclpL-fabZ1.

SEQ ID NOs: 74 and 75 are primers for amplification of PfabZ1 left homologous arm.

SEQ ID NOs: 76 and 77 are primers for amplification of PfabZ1 right homologous arm.

SEQ ID NOs: 78 and 79 are primers for amplification of PclpL.

SEQ ID NOs: 80-81 are used for confirmation of strain PN0512ΔpyrF_PclpL-fabZ.

SEQ ID NO: 82 encodes cydA promoter region.

SEQ ID NO: 83 encodes atpB promoter region.

SEQ ID NO: 84 encodes agrB promoter region.

SEQ ID NOs: 85 and 86 are primers for amplification for IdhL from L. plantarum.

SEQ ID NO: 87 is nucleic acid sequence encoding pFP988.

SEQ ID NOs: 88 and 89 are primers for amplification of CmR from pC194.

SEQ ID NOs: 90 and 91 are primers for construction of P11.

SEQ ID NOs: 92 and 93 are primers for amplification for IdhL promoter from L. plantarum ATCC BAA-793.

### DETAILED DESCRIPTION OF THE INVENTION

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “concentration” is used to as a measure of how much of a given substance is mixed with another substance. For example the concentration of butanol is expressed as % (weight/volume). In another example the concentration of stearic acid fed in the growth media is expressed as mg/liter. In another example, the concentration of C18:0 fatty acid in the bacterial cell membrane is measured as molar % in comparison to total fatty acid content in the membrane that includes both saturated and unsaturated fatty acids. For comparison purposes internal controls are included and same unit of concentration is used between control and test measurements.

“Tolerance” is defined as the ability of a cell to survive in an environment and may be expressed as a multiplication factor or percentage of a nominal value that reflects baseline environment. The nominal value may be defined in terms of number of cells, rate of cell growth, rate of decline in the rate of cell death, rate of cell division or other measures of cellular viability and survival. In one example, the increased tolerance to butanol in this invention was measured as an increase in growth yield by a factor of 1.57 in Example 2, Table 7.

“Genetic modification” refers to inheritable changes or alterations introduced in the genetic code of a cell. These changes or modifications may be randomly generated or by rational design. The changes may span a minimum of 1 nucleotide or can be a contiguous block of nucleotides or non-contiguous nucleotide regions spanning significant portions of an organism's genome.

The term “gene” refers to a nucleic acid fragment that is capable of being expressed as a specific protein, optionally including regulatory sequences preceding (5′ non-coding sequences) and following (3′ non-coding sequences) the coding sequence. The term “native” refers to a gene of natural occurrence in a cell in contrast to a foreign gene introduced by artificial intervention. “Modified gene” refers to any gene that is not identical to the native gene and may comprise regulatory and coding sequences that are not found in tandem in nature. Accordingly, a modified gene may comprise regulatory sequences and coding sequences that are derived from different sources, or regulatory sequences and coding sequences derived from the same source, but arranged in a manner different than that found in nature. A modified gene may also comprise a coding sequence derived from the native gene but altered by random mutagenesis or rational design. A modified gene may be a chimera (sometimes knows as a mosaic), comprising domains swapped from two or more genes. “Endogenous gene” is of the same cellular origin as “native gene” as opposed to exogenous or foreign gene which is derived from the genome of a genetically distinct cell. A “foreign gene” or “heterologous gene” refers to a gene not normally found in the host organism, but that is introduced into the host organism by genetic modification or manipulation, or is present in a host cell but is modified or manipulated so as to affect its regulation.

The term “down-regulated” describes functional state of a gene, in which the level of expression of gene is reduced. The down regulation may be achieved by modification of the genetic structure or by alteration of environmental conditions.

The term “disruption” means interruption of functional unit of a gene to block gene function.

The term “expression”, as used herein refers to transcription of RNA including antisense RNA, reverse transcription or translation of mRNA into a polypeptide or a combination thereof.

The term “episomal” is descriptive of a genetic element or a nucleotide sequence present on an episome. The episome is an extrachromosomal DNA element. DNA is deoxyribonucleic acid.

The term “coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The terms “plasmid” (or “vector”) refer to an extra chromosomal element often carrying genes which are not part of the central metabolism of the cell, and exist most commonly in the form of circular double-stranded DNA fragments. Such elements may be autonomously replicating sequences or genome integrating sequences; linear or circular; single- or double-stranded DNA or RNA; and may be isolated or synthetically derived from any source in which a number of nucleotide sequences have been joined or recombined into a unique construction which is capable of introducing a promoter fragment and DNA sequence for a selected gene product along with appropriate 3′ downstream regulatory sequence into a cell. “Transformation vector” refers to a specific vector containing a foreign gene and having elements in addition to the foreign gene that facilitates transformation of a particular host cell.

The term “codon-optimized” as it refers to genes or coding regions of nucleic acid molecules for transformation of various hosts, refers to the alteration of codons in a gene or coding regions of the nucleic acid molecules to reflect the typical codon usage of the host organism without altering the polypeptide encoded by the DNA.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences is performed using the Clustal method of alignment (Higgins and Sharp, CABIOS. 5:151-153 (1989)) with default parameters (GAP PENALTY=10, GAP LENGTH PENALTY=10), unless otherwise specified. Default parameters for pairwise alignments using the Clustal method are: KTUPLE 1, GAP PENALTY=3, WINDOW=5 and DIAGONALS SAVED=5.

Contemplated herein are nucleic acid sequences that encode polypeptides that are at least about 70% identical, preferably at least about 75% identical, and more preferably at least about 80% identical to the amino acid sequences reported herein. Preferred nucleic acid fragments encode amino acid sequences that are about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. In embodiments, suitable nucleic acid fragments encode a polypeptide having at least 50 amino acids, preferably at least 100 amino acids, more preferably at least 150 amino acids, still more preferably at least 200 amino acids, and most preferably at least 250 amino acids.

A nucleic acid molecule may hybridize to another nucleic acid molecule, such as a cDNA, genomic DNA, or RNA molecule, when a single-stranded form of the nucleic acid molecule can anneal to the other nucleic acid molecule under the appropriate conditions of temperature and solution ionic strength. Given the nucleic acid sequences described herein, one of skill in the art can identify substantially similar nucleic acid fragments that may encode proteins having similar activity. As used herein substantially similar enzymes will refer to enzymes belonging to a family of proteins in the art known to share similar structures and function. It is well within the skill of one in the art to identify substantially similar proteins given a known structure. Typical methods to identify substantially similar structures will rely upon known sequence information (nucleotide sequence and/or amino acid sequences) and may include PCR amplification, nucleic acid hybridization, and/or sequence identity/similarity analysis (e.g., sequence alignments between partial and/or complete sequences and/or known functional motifs associated with the desired activity).

The term “homology” refers to the structural relationship among genetic elements whereby there is some extent of similarity in the nucleotide and amino acid sequences, typically due to descent from a common ancestral origin. The term “ortholog” or “orthologous sequences” refers herein to a relationship where sequence divergence follows speciation (i.e., homologous sequences in different species arose from a common ancestral gene during speciation). In contrast, the term “paralogous” refers to homologous sequences within a single species that arose by gene duplication. One skilled in the art will be familiar with techniques required to identify homologous, orthologous and paralogous sequences.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein, “default values” will mean any set of values or parameters (as set by the software manufacturer) which originally load with the software when first initialized.

Standard recombinant DNA and molecular cloning techniques used here are well known in the art and are described by Sambrook, J., Fritsch, E. F. and Maniatis, T. Molecular Cloning: A Laboratory Manual, 2^{nd} ed.; Cold Spring Harbor Laboratory: Cold Spring Harbor, N.Y., 1989 (hereinafter “Maniatis”); and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by Greene Publishing and Wiley-Interscience, 1987.

The term “unsaturated fatty acid biosynthetic pathway” refers to a series of steps in which one molecular species is converted to another to serve as starting reactant in the next step resulting ultimately in production of unsaturated fatty acid(s).

The term “UFA” is unsaturated fatty acid. In an unsaturated fatty acid one or more alkenyl functional groups exist along the chain, with each alkene substituting a single-bonded “—CH2-CH2-” part of the chain with a double-bonded “—CH═CH—” portion (that is, a carbon double-bonded to another carbon). Some examples of UFA used in this invention are C16:1 and C18:1.

The term “saturated fatty acids” are fatty acids with saturated “—C—C—” bonds along the chain in their molecular structure.

The term “membrane” refers to the cellular fraction comprising phospholipid bilayers.

The term “FAME” refers to Fatty Acid Methyl Ester analysis.

The term “feeding” refers to providing in the growth medium.

“5-FOA” is a toxic pyrimidine analog that is incorporated via the de novo biosynthetic pathway. Resistance to 5-FOA can be achieved by mutation of pathway genes (Boeke, J., LaCroute, F., and Fink, G., A positive selection for mutants lacking orotidine-5′-phosphate decarboxylase activity in yeast: 5-fluoroorotic acid resistance, 1984, Mol. Gen. Genet. 197:345-346).

The term “fabZ1” refers to a gene that encodes a FabZ1 protein having activity for isomerizationof trans-2-decenoyl-ACP to cis-3-decenoyl-ACP and β-hydroxyacyl-(Acyl Carrier Protein) dehydratase activity.

The term FabZ1 refers herein to bifunctional proteins that catalyze β-hydroxyacyl-(Acyl Carrier Protein) dehydratase activity (which is classified as EC 4.2.1) and isomerization of trans-2-decenoyl-ACP to cis-3-decenoyl-ACP activity.

The term “trans-2-decenoyl-ACP” is same as trans-2-decenoyl-Acyl Carrier Protein. The term “cis-3-decenoyl-ACP” is same as cis-3-decenoyl-Acyl Carrier Protein.

The enzymes catalyzing J3-hydroxyacyl-(Acyl Carrier Protein) dehydratase activity are assigned Enzyme Commission Numbers based on the carbon chain length of the substrate as shown in Table 5.

TABLE 5
A list of EC (Enzyme Commission) numbers that describe
activities catalyzed by the enzyme β-hydroxyacyl-(Acyl Carrier Protein)
dehydratase encoded by any of the genes selected from fabA, fabM, fabN,
fabZ and fabZ1. The recommended names and synonyms are retrieved
from the BRENDA database.

| EC | Biological | Recommended | |
|---|---|---|---|
| Number | Sources | Name | Synonyms |
| 4.2.1.58 | Escherichia coli, | Crotonoyl-[acyl- | 3-Hydroxybutyryl |
| | Shewanella | carrier-protein] | Acyl Carrier Protein |
| | piezotolerans | hydratase | dehydratase |
| | (strain WP3/JCM | | |
| | 13877) | | |
| 4.2.1.59 | Escherichia coli | 3-Hydroxyoctanoyl- | D-3- |
| | | [acyl-carrier-protein] | Hydroxyoctanoyl- |
| | | dehydratase | Acyl Carrier Protein |
| | | | dehydratase |
| 4.2.1.60 | Escherichia coli | 3-Hydroxydecanoyl- | 3-Hydroxydecanoyl- |
| | Brevibacterium | [acyl-carrier-protein] | Acyl Carrier Protein |
| | ammoniagenes, | dehydratase | dehydratase, beta- |
| | Aerobacter | | Hydroxyacyl-Acyl |
| | aerogenes | | Carrier Protein |
| | | | dehydratase |
| 4.2.1.61 | Escherichia coli | 3-Hydroxypalmitoyl- | D-3- |
| | | [acyl-carrier-protein] | Hydroxypalmitoyl- |
| | | dehydratase | [Acyl Carrier |
| | | | Protein] |
| | | | dehydratase |

Proteins having activity for isomerization of trans-2-decenoyl-Acyl Carrier Protein to cis-3-decenoyl-Acyl Carrier Protein and a β-hydroxyacyl-(Acyl Carrier Protein) dehydratase activity are encoded by genes that have been designated by any of the several names for example fabA, fabN, fabM, fabZ and fabZ1.

Escherichia coli produces straight-chain saturated fatty acids (SFA) and monounsaturated fatty acids. In E. coli unsaturated fatty acid (UFA) biosynthesis synthesis requires the action of two gene products, the essential isomerase/dehydratase encoded by fabA and an elongation condensing enzyme encoded by fabB. In E. coli, the gene fabA encodes beta-hydroxydecanoyl-Acyl Carrier Protein dehydratase.

Streptococcus pneumoniae lacks both genes and instead employs a single enzyme with only an isomerase function encoded by the fabM gene. The fabN gene of Enterococcus faecalis, coding for a dehydratase/isomerase, complements the growth of S. pneumoniae fabM mutants.

The products of the genes fabA, fabN, fabM, fabZ and fabZ1 and their respective orthologs comprise at a minimum activity for isomerization of trans-2-decenoyl-Acyl Carrier Protein to cis-3-decenoyl-Acyl Carrier Protein and optionally a β-hydroxyacyl-(Acyl Carrier Protein) dehydratase activity. A biological source of activity for isomerization of trans-2-decenoyl-Acyl Carrier Protein to cis-3-decenoyl-Acyl Carrier Protein may optionally have a β-hydroxyacyl-(Acyl Carrier Protein) dehydratase activity and may include an amino acid sequence of the enzyme or a nucleotide sequence which may be used to express a protein with desired isomerization activity. The biological sources of activity for isomerization of trans-2-decenoyl-Acyl Carrier Protein to cis-3-decenoyl-Acyl Carrier Protein may also be an organism which comprises J3-hydroxyacyl-(Acyl Carrier Protein) dehydratase activity.

Accordingly nucleotide and amino acid sequences associated with activity for isomerization of trans-2-decenoyl-Acyl Carrier Protein but are not limited to the sequences derived from Lactobacillus plantarum (GI:28271195, GI: 254556570, SEQ ID NOs: 94, 96, 99), Lactobacillus sakei (GI: 78610067, SEQ ID NO:95), Lactococcus lactis (GI:12723452, SEQ ID NO: 97), Leuconostoc citreum (GI:170016657, SEQ ID NO: 98), Lactobacillus ultunensis (GI: 227892760, SEQ ID NO: 100) and Enterococcus faecalis (NP_{—}814076, SEQ ID NO: 102), Escherichia coli (GI:242376769, SEQ ID NO: 120), Lactobacillus reuteri (GI:133930504, SEQ ID NO: 122), Agrobacterium radiobacter K84 (GI:221721763, SEQ ID NO: 124), Streptococcus mutans UA159 (GI: 50253369, SEQ ID NO: 124), and orthologs thereof. Refer to Table 4 for more examples (SEQ ID NOs: 94-127). Several other biological sources are described in Table 5 as well.

The term “butanol” as used herein, refers to 1-butanol, 2-butanol, isobutanol, or mixtures thereof.

The terms “butanol tolerant bacterial strain” or “tolerant” when used in reference to a modified bacterial strain of the invention, refers to a modified bacterium that shows better growth in the presence of butanol than the parent strain from which it is derived. Such a strain may also be characterized by enhanced survival (both in numbers and longevity), enhanced production of butanol and intermediates.

As used herein, the term “wild-type” or parent is a relational term, and refers to a cell which has not been modified as opposed to the cell (or strain) that has been modified to prepare a genetic construct of expected outcome. For example in the case of a modified bacterial cell (or strain) that shows increased tolerance to butanol compared to the strain from which it is derived, the latter is wild-type or parent strain with respect to the modified strain. In another example, BP15 is parent or wild-type strain with respect to BP63 strain.

“Biosynthetic pathway” refers to a series of steps in which one molecular species is converted to another to serve as starting reactant in the next step. A biosynthetic pathway in a cell is a part of a highly interconnected network of reactions.

“Butanol biosynthetic pathway” refers to a series of steps in which one molecular species is converted to another to serve as starting reactant in the next step with the ultimate production of butanol. Consistent with this definition, the term “butanol biosynthetic pathway” refers to an enzyme pathway to produce 1-butanol, 2-butanol, or isobutanol.

The term “1-butanol biosynthetic pathway” refers to an enzyme pathway to produce 1-butanol from acetyl-coenzyme A (acetyl-CoA).

The term “2-butanol biosynthetic pathway” refers to an enzyme pathway to produce 2-butanol from pyruvate.

The term “isobutanol biosynthetic pathway” refers to an enzyme pathway to produce isobutanol from pyruvate.

The term “acetyl-CoA acetyltransferase” refers to an enzyme that catalyzes the conversion of two molecules of acetyl-CoA to acetoacetyl-CoA and coenzyme A (CoA). Preferred acetyl-CoA acetyltransferases are acetyl-CoA acetyltransferases with substrate preferences (reaction in the forward direction) for a short chain acyl-CoA and acetyl-CoA and are classified as E.C. 2.3.1.9 [Enzyme Nomenclature 1992, Academic Press, San Diego]; although, enzymes with a broader substrate range (E.C. 2.3.1.16) will be functional as well. Acetyl-CoA acetyltransferases are available from a number of sources, for example, Escherichia coli (GenBank NOs: NP_{—}416728, NC_{—}000913, Clostridium acetobutylicum (GenBank NOs: NP_{—}349476.1 (SEQ ID NO:2), NC_{—}003030; NP_{—}149242 (SEQ ID NO:4), NC_{—}001988), Bacillus subtilis (GenBank Nos: NP_{—}390297, NC_{—}000964), and Saccharomyces cerevisiae (GenBank Nos: NP_{—}015297, NC_{—}001148).

The term “3-hydroxybutyryl-CoA dehydrogenase” refers to an enzyme that catalyzes the conversion of acetoacetyl-CoA to 3-hydroxybutyryl-CoA. 3-Hydroxybutyryl-CoA dehydrogenases may be reduced nicotinamide adenine dinucleotide (NADH)-dependent, with a substrate preference for (S)-3-hydroxybutyryl-CoA or (R)-3-hydroxybutyryl-CoA and are classified as E.C. 1.1.1.35 and E.C. 1.1.1.30, respectively. Additionally, 3-hydroxybutyryl-CoA dehydrogenases may be reduced nicotinamide adenine dinucleotide phosphate (NADPH)-dependent, with a substrate preference for (S)-3-hydroxybutyryl-CoA or (R)-3-hydroxybutyryl-CoA and are classified as E.C. 1.1.1.157 and E.C. 1.1.1.36, respectively. 3-Hydroxybutyryl-CoA dehydrogenases are available from a number of sources, for example, C. acetobutylicum (GenBank NOs: NP_{—}349314 (SEQ ID NO:6), NC_{—}003030), B. subtilis (GenBank NOs: AAB09614, U29084), Ralstonia eutropha (GenBank NOs: ZP_{—}0017144, NZ_AADY01000001, Alcaligenes eutrophus (GenBank NOs: YP_{—}294481, NC_{—}007347), and A. eutrophus (GenBank NOs: P14697, J04987).

The term “crotonase” refers to an enzyme that catalyzes the conversion of 3-hydroxybutyryl-CoA to crotonyl-CoA and H₂O. Crotonases may have a substrate preference for (S)-3-hydroxybutyryl-CoA or (R)-3-hydroxybutyryl-CoA and are classified as E.C. 4.2.1.17 and E.C. 4.2.1.55, respectively. Crotonases are available from a number of sources, for example, E. coli (GenBank NOs: NP_{—}415911 (SEQ ID NO:8), NC_{—}000913), C. acetobutylicum (GenBank NOs: NP_{—}349318, NC_{—}003030), B. subtilis (GenBank NOs: CAB13705, Z99113), and Aeromonas caviae (GenBank NOs: BAA21816, D88825).

The term “butyryl-CoA dehydrogenase”, also called trans-enoyl CoA reductase (TER), refers to an enzyme that catalyzes the conversion of crotonyl-CoA to butyryl-CoA. Butyryl-CoA dehydrogenases may be either NADH-dependent, NADPH-dependent, or flavin-dependent and are classified as E.C. 1.3.1.44, E.C. 1.3.1.38, and E.C. 1.3.99.2, respectively. Butyryl-CoA dehydrogenases are available from a number of sources, for example, C. acetobutylicum (GenBank NOs: NP_{—}347102 (SEQ ID NO:10), NC_{—}003030), Euglena gracilis (GenBank NOs: □5EU90, AY741582), Streptomyces collinus (GenBank NOs: AAA92890, U37135), and Streptomyces coelicolor (GenBank NOs: CAA22721, AL939127).

The term “butyraldehyde dehydrogenase” refers to an enzyme that catalyzes the conversion of butyryl-CoA to butyraldehyde, using NADH or NADPH as cofactor. Butyraldehyde dehydrogenases include those known as E.C. 1.2.1.10 and those with a preference for NADH are known as E.C. 1.2.1.57 and are available from, for example, Clostridium beijerinckii (GenBank NOs: AAD31841 (SEQ ID NO:12), AF157306) and C. acetobutylicum (GenBank NOs: NP_{—}149325, NC_{—}001988).

The term “1-butanol dehydrogenase” refers to an enzyme that catalyzes the conversion of butyraldehyde to 1-butanol. 1-butanol dehydrogenases are a subset of the broad family of alcohol dehydrogenases. 1-butanol dehydrogenase may be NADH- or NADPH-dependent. 1-butanol dehydrogenases are available from, for example, C. acetobutylicum (GenBank NOs: NP_{—}149325, NC_{—}001988; NP_{—}349891 (SEQ ID NO:14), NC_{—}003030; and NP_{—}349892 (SEQ ID NO:16), NC_{—}003030) and E. coli (GenBank NOs: NP_{—}417484, NC_{—}000913).

The term “acetolactate synthase”, also known as “acetohydroxy acid synthase”, refers to a polypeptide (or polypeptides) having an enzyme activity that catalyzes the conversion of two molecules of pyruvic acid to one molecule of alpha-acetolactate. Acetolactate synthase, known as EC 2.2.1.6 (formerly 4.1.3.18) (Enzyme Nomenclature 1992, Academic Press, San Diego) may be dependent on the cofactor thiamin pyrophosphate for its activity. Suitable acetolactate synthase enzymes are available from a number of sources, for example, Bacillus subtilis (GenBank Nos: AAA22222 NCBI (National Center for Biotechnology Information) amino acid sequence, L04470 NCBI nucleotide sequence), Klebsiella terrigena (GenBank Nos: AAA25055, L04507), and Klebsiella pneumoniae (GenBank Nos: AAA25079 (SEQ ID NO:20), M73842 (SEQ ID NO:19).

The term “acetolactate decarboxylase” refers to a polypeptide (or polypeptides) having an enzyme activity that catalyzes the conversion of alpha-acetolactate to acetoin. Acetolactate decarboxylases are known as EC 4.1.1.5 and are available, for example, from Bacillus subtilis (GenBank Nos: AAA22223, L04470), Klebsiella terrigena (GenBank Nos: AAA25054, L04507) and Klebsiella pneumoniae (SEQ ID NO:18 (amino acid) SEQ ID NO:17 (nucleotide)).

The term “butanediol dehydrogenase” also known as “acetoin reductase” refers to a polypeptide (or polypeptides) having an enzyme activity that catalyzes the conversion of acetoin to 2,3-butanediol. Butanediol dehydrogenases are a subset of the broad family of alcohol dehydrogenases. Butanediol dehydrogenase enzymes may have specificity for production of R- or S-stereochemistry in the alcohol product. S-specific butanediol dehydrogenases are known as EC 1.1.1.76 and are available, for example, from Klebsiella pneumoniae (GenBank Nos: BBA13085 (SEQ ID NO:22), D86412. R-specific butanediol dehydrogenases are known as EC 1.1.1.4 and are available, for example, from Bacillus cereus (GenBank Nos. NP_{—}830481, NC_{—}004722; AAP07682, AE017000), and Lactococcus lactis (GenBank Nos. AAK04995, AE006323).

The term “butanediol dehydratase”, also known as “diol dehydratase” or “propanediol dehydratase” refers to a polypeptide (or polypeptides) having an enzyme activity that catalyzes the conversion of 2,3-butanediol to 2-butanone, also known as methyl ethyl ketone (MEK). Butanediol dehydratase may utilize the cofactor adenosyl cobalamin. Adenosyl cobalamin-dependent enzymes are known as EC 4.2.1.28 and are available, for example, from Klebsiella oxytoca (GenBank Nos: BAA08099 (alpha subunit) (SEQ ID NO:24), BAA08100 (beta subunit) (SEQ ID NO:26), and BBA08101 (gamma subunit) (SEQ ID NO:28), (Note all three subunits are required for activity), D45071).

The term “2-butanol dehydrogenase” refers to a polypeptide (or polypeptides) having an enzyme activity that catalyzes the conversion of 2-butanone to 2-butanol. 2-butanol dehydrogenases are a subset of the broad family of alcohol dehydrogenases. 2-butanol dehydrogenase may be NADH- or NADPH-dependent. The NADH-dependent enzymes are known as EC 1.1.1.1 and are available, for example, from Rhodococcus ruber (GenBank Nos: CAD36475 (SEQ ID NO:30), AJ491307 (SEQ ID NO:29)). The NADPH-dependent enzymes are known as EC 1.1.1.2 and are available, for example, from Pyrococcus furiosus (GenBank Nos: AAC25556, AF013169).

The term “acetohydroxy acid isomeroreductase” or “acetohydroxy acid reductoisomerase” refers to an enzyme that catalyzes the conversion of acetolactate to 2,3-dihydroxyisovalerate using NADPH (reduced nicotinamide adenine dinucleotide phosphate) as an electron donor. Preferred acetohydroxy acid isomeroreductases are known by the EC number 1.1.1.86 and sequences are available from a vast array of microorganisms, including, but not limited to, Escherichia coli (GenBank Nos: NP_{—}418222 (SEQ ID NO:32), NC_{—}000913 (SEQ ID NO:31)), Saccharomyces cerevisiae (GenBank Nos: NP_{—}013459, NC_{—}001144), Methanococcus maripaludis (GenBank Nos: CAF30210, BX957220), and Bacillus subtilis (GenBank Nos: CAB14789, Z99118).

The term “acetohydroxy acid dehydratase” refers to an enzyme that catalyzes the conversion of 2,3-dihydroxyisovalerate to α-ketoisovalerate. Preferred acetohydroxy acid dehydratases are known by the EC number 4.2.1.9. These enzymes are available from a vast array of microorganisms, including, but not limited to, E. coli (GenBank Nos: YP_{—}026248 (SEQ ID NO:34), NC_{—}000913 (SEQ ID NO:33)), S. cerevisiae (GenBank Nos: NP_{—}012550, NC_{—}001142), M. maripaludis (GenBank Nos: CAF29874, BX957219), and B. subtilis (GenBank Nos: CAB14105, Z99115).

The term “branched-chain α-keto acid decarboxylase” refers to an enzyme that catalyzes the conversion of α-ketoisovalerate to isobutyraldehyde and CO₂. Branched-chain α-keto acid decarboxylases are known by the EC number 4.1.1.1 or EC number 4.1.1.72 and are available from a number of sources, including, but not limited to, Lactococcus lactis (GenBank Nos: AAS49166, AY548760; CAG34226 (SEQ ID NO:36), AJ746364, Salmonella typhimurium (GenBank Nos: NP_{—}461346, NC_{—}003197), and Clostridium acetobutylicum (GenBank Nos: NP_{—}149189, NC_{—}001988).

The term “branched-chain alcohol dehydrogenase” refers to an enzyme that catalyzes the conversion of isobutyraldehyde to isobutanol. Preferred branched-chain alcohol dehydrogenases are known by the EC number 1.1.1.265, but may also be classified under other alcohol dehydrogenases (specifically, EC 1.1.1.1 or 1.1.1.2). These enzymes utilize NADH (reduced nicotinamide adenine dinucleotide) and/or NADPH as electron donor and are available from a number of sources, including, but not limited to, S. cerevisiae (GenBank Nos: NP_{—}010656, NC_{—}001136; NP_{—}014051, NC_{—}001145), E. coli (GenBank Nos: NP_{—}417484 (SEQ ID NO:38), NC_{—}000913 (SEQ ID NO:37)), and C. acetobutylicum (GenBank Nos: NP_{—}349892, NC_{—}003030).

The present invention provides a method for increasing the tolerance of a bacterial cell to butanol comprising increasing the concentration of saturated fatty acids in the membrane of the bacterial cell. As demonstrated herein, such cells have increased tolerance to butanol as compared with cells that lack the membrane fatty acid composition modification. Such cells may comprise a butanol biosynthetic pathway and butanol produced using the cells described in this invention may be used as an energy source alternative to fossil fuels.

An increase in saturated fatty acid composition of bacterial cell membrane may be accomplished by feeding saturated fatty acids. In one embodiment, cells are grown in media comprising at least one saturated fatty acid. In embodiments, saturated fatty acid is present in the media in an amount ranging from about 30-500 mg/L. In embodiments, saturated fatty acid is present in the media in an amount of at least about 30 mg/L, at least about 50 mg/L, at least about 100 mg/L, at least about 200 mg/L, at least about 400 mg/L, or about 500 mg/L.

An increase in saturated fatty acid composition of bacterial cell membrane relative to unsaturated fatty acid composition may be accomplished by genetically modifying the cell to modulate the expression of at least one gene involved in unsaturated trans fatty acid biosynthesis, such as one encoding activity for isomerization of trans-2-decenoyl-Acyl Carrier Protein to cis-3-decenoyl-Acyl Carrier Protein.

In one embodiment, the cells of present invention are genetically modified and have an increased tolerance to butanol as compared with cells that lack the genetic modification, and may be used to produce butanol, a source of energy alternative to fossil fuels. In embodiments, the genetic modification provides for increased concentration of saturated fatty acids in the cell membrane.

In one embodiment the bacterial cell comprises a genetic modification in a gene of an unsaturated fatty acid biosynthetic pathway. In embodiments, the gene of an unsaturated fatty acid biosynthetic pathway is any one or more of the genes selected from the group consisting of fabA, fabM, fabN, fabZ and fabZ1. In embodiments, the gene of an unsaturated fatty acid biosynthetic pathway encodes a protein that catalyzes isomerization of trans-2-decenoyl-ACP to cis-3-decenoyl-ACP.

In one embodiment the butanol tolerant bacterial cell comprises decreased or eliminated expression of a gene of an unsaturated fatty acid biosynthetic pathway, for example, the fabZ1 gene. In another embodiment the bacterial cell comprises a genetic modification resulting in an increased concentration of saturated fatty acids in the membrane. Suitable genetic modifications include, but are not limited to, deletion of a gene of an unsaturated fatty acid biosynthetic pathway or expression of a gene of an unsaturated fatty acid biosynthetic pathway operably linked to a promoter which provides reduced expression, or a combination thereof.

In embodiments, the bacterial cell comprises a genetic modification whereby a gene of an unsaturated fatty acid biosynthetic pathway, such as, for example, the fabZ1 gene is operably linked to a non-native promoter. In embodiments, the promoter provides for reduced expression of the gene of an unsaturated fatty acid biosynthetic pathway, such as fabZ1, as compared to the parent strain. Suitable promoters are known in the art and include, but are not limited to, clpL, cydA, agrB, or atpB from L. plantarum, The gene of an unsaturated fatty acid biosynthetic pathway operably linked to a promoter that provides for reduced expression of the gene, for example the fabZ1 gene, may be located on an extra-chromosomal element or integrated within the genome. In embodiments, the genetic modification comprises deletion of a gene of an unsaturated fatty acid biosynthetic pathway is deleted. In other embodiments, the genetic modification comprises deletion of the gene of an unsaturated fatty acid biosynthetic pathway from the chromosome, and, in embodiments, the cell further comprises an genetic modification whereby the deleted gene or an alternate gene of an unsaturated fatty acid biosynthetic pathway is expressed on an extra-chromosomal element. The fabZ1 gene may be substituted by any of the genes selected from fabA, fabM, fabN, fabZ and fabZ1 wherein the product of these genes catalyzes β-hydroxyacyl-ACP dehydratase activity.

In one embodiment the butanol tolerant bacterial cell is selected from the group consisting of Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Lactococcus, Pediococcus, and Leuconostoc.

In one specific instance the butanol tolerant bacterial cell is a lactobacillus cell having a genetic modification in a gene selected from the group consisting of fabA, fabM, fabN, fabZ and fabZ1 and having at least about a 10% increase in total cell membrane saturated fatty acids as compared with a wild-type lactobacillus cell.

In one embodiment, the activity of an enzyme with J3-hydroxyacyl-ACP dehydratase activity and trans-2-decenoyl-ACP to cis-3-decenoyl-ACP isomerization activity in a Lactobacillus plantarum cell is decreased. Methods of creating mutants for the purpose of identification of such genes in a desirable organism are described by markerless deletions made through homologous recombination.

Provided herein is a recombinant bacterial cell that does not naturally produce butanol and has been:

(i) modified to have increased molar ratios of saturated fatty acids in total fatty acid composition of the bacterial membranes as compared with the unmodified bacterial cell, and

(ii) engineered to express a butanol biosynthetic pathway.

The butanol tolerant bacterial cells provided herein may be used for the production of butanol, wherein the butanol tolerant bacterial cell comprises:
a) a butanol biosynthetic pathway,
b) a cell membrane having at least about a 10% increase in total cell membrane saturated fatty acid content as compared with a parent bacterial cell;

wherein the butanol biosynthetic pathway comprises at least one gene that is heterologous to the bacterial cell.

This invention also describes a bacterial cell having at least about a 25% increase in total cell membrane saturated fatty acid content as compared with a parent bacterial cell.

Butanol Tolerance In Butanol Non-Producing Bacteria—Membrane Composition

Disclosed herein is the discovery that an increase in the saturated fatty acid content of the membrane of a bacterial cell that does not naturally produce butanol increases butanol tolerance of the cell. Any bacteria that does not naturally produce butanol may have increased butanol tolerance through an increase in membrane saturated fatty acid composition. Examples include, but are not limited to, bacterial cells of Zymomonas, Escherichia, Salmonella, Rhodococcus, Pseudomonas, Bacillus, Lactobacillus, Enterococcus, Pediococcus, Alcaligenes, Klebsiella, Paenibacillus, Arthrobacter, Corynebacterium, Leuconostoc, Clostridium and Brevibacterium. Examples of specific bacterial cells include: Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Zymomonas mobilis, Lactococcus lactis and Bacillus subtilis.

Increasing Membrane Saturated Fatty Acids

Provided herein is a method of increasing the tolerance of a bacterial cell to butanol comprising feeding at least one saturated fatty acid. Also provided is a bacterial cell having at least about 10%, at least about 20%, or at least about 25% increase in total cell membrane saturated fatty acid content as compared with a parent bacterial cell. The amount of saturated fatty acids in the membrane may be increased with respect to the amounts of other types of fatty acids by methods including, but not limited to, A) feeding the cells a saturated fatty acid that will result in an increase in membrane saturated fatty acid, B) genetic modification resulting in (i) increasing the membrane saturated trans fatty acid composition and/or (ii) increasing the saturated/unsaturated fatty acid ratio (Ratio^{SFA/UFA}; see, for example, Example 1), or C) an integrated approach involving both A) and B). Methods applying an integrated approach include, for example feeding saturated fatty acids to a genetically modified strain that has altered expression of unsaturated fatty acid pathway genes such that total unsaturated acid present in the cell membrane is reduced. Suitable methods are described and/or exemplified herein (see Examples). Method of calculating Ratio^{SFN/UFA} is described in Example 1.

Fatty acids that may be fed to cells to increase membrane saturated fatty acid composition include, for example, C14:0 (Trivial Name: Myristic Acid; IUPAC name: Tetradecanoic Acid, CAS Registry Number: 544-63-8), C15:0; (IUPAC name: Pentadecanoic Acid, CAS Registry Number: 5502-94-3), C16:0 (Trivial Name: Palmitic Acid; IUPAC name: Hexadecanoic Acid, CAS Registry Number: 57-10-3), C17:0 (IUPAC name: Heptadecanoic Acid, CAS Registry Number: 506-12-7), C18:0 (Trivial Name: Stearic acid; IUPAC name: Octadecanoic Acid, CAS Registry Number: 57-11-4), C19:0 (IUPAC name: Nonadecanoic Acid, CAS Registry Number: 646-30-0) and C20:0 (Trivial Name: Arachidic Acid; IUPAC name: Icosanoic Acid, CAS Registry Number: 506-30-9).

Availability of Fatty Acids

The fatty acids (saturated and unsaturated) with even- and odd-carbon chains are commercially available, and may be purchased as kits or individually from Sigma-Aldrich. Dihydrosterculic acid (CAS# 4675-61-0, cyc-C19:0, 9-) for membrane fatty acid analysis is commercially available, and may be purchased from INDOFINE Chemical Company (Hillsborough, N.J. 08844).

Molar Ratio of Saturated Fatty Acids to Unsaturated Fatty Acids

The ratio of total saturated fatty acids to unsaturated (C16:0 and C18:0) to (C16:1 and C18:1, cis) may be determined according to the example calculations below:

Ratio^{SFA/UFA}=(Molar % C16:0+Molar % C18:0)÷(Molar % C16:1+Molar % C18:1).

In this example, the C16:0 and C18:0 (Molar %) content of saturated fatty acids was divided by a sum of C16:1 and C18:1, cis content (Molar %) of unsaturated acid in order to calculate saturated/unsaturated fatty acid composition ratios in the membrane. One of skill in the art will readily appreciate the application of the calculation for other saturated fatty acids (e.g. C14:0 or C20:0) and the corresponding unsaturated fatty acids (e.g. C14:1 or C20:1) to determine saturated/unsaturated fatty acid composition ratios.

Altering Fatty Acids in the Membrane by Genetic Manipulation

Contemplated herein is a method to increase saturated fatty acids in the membrane comprising reducing expression of genes encoding proteins responsible for unsaturated fatty acid biosynthesis. In one embodiment of the present invention a previously uncharacterized unsaturated fatty acid biosynthetic pathway in L. plantarum has been genetically modified and successfully manipulated for regulating unsaturated fatty acid biosynthesis.

The pathway of unsaturated fatty acid (UFA) biosynthesis has been described in E. coli (Rock, C. O., and Cronan, J. E. (1996). Escherichia coli as a model for the regulation of dissociable (type II) fatty acid biosynthesis. Biochim Biophys Acta 1302: 1-16) and is considered the paradigm for anaerobic unsaturated fatty acids biosynthesis. Two proteins FabA and FabB are required for generation of a cis double bond during fatty acid elongation. E. coli strains mutated in fabA or fabB require unsaturated fatty acid for growth. Streptococcus mutans has an alternative pathway for unsaturated fatty acid biosynthesis utilizing an enzyme, FabM (Fozo, E. M. and Quivey Jr., R. G. (2004) Journal of Bacteriology, 186(13): 4152-4158). In Streptococcus pneumoniae, FabM is shown to be responsible for the production of monounsaturated fatty acids (Marrakchi et. al. (2002) J. Biol. Chem. 277:44809-44816,). Altabe et al (2007) have shown that the fabN gene of Enterococcus faecalis, which is involved in synthesis of unsaturated fatty acids may be used to complement the function of fabM (Journal of Bacteriology. 189 (22): 8139-8144). Wang and Cronan (2004) have shown that Enterococcus faecalis fabZ1 (fabZ1 of E. faecalis is same as fabN) can functionally replace the E. coli fabZ1 (J. Biol. Chem. 279: 34489-95). Thus it is reasonable that the genes fabA, fabM, fabN, fabZ and fabZ1 all encoding at a minimum activity for isomerization of trans-2-decenoyl-Acyl Carrier Protein to cis-3-decenoyl-Acyl Carrier Protein and optionallyl β-hydroxyacyl-[Acyl Carrier Protein] dehydratase activity can be functionally substituted across diverse bacterial genera for complementing the deficiency for isomerization of trans-2-decenoyl-Acyl Carrier Protein to cis-3-decenoyl-Acyl Carrier Protein.

L. plantarum, like E. faecalis, has two genes encoding proteins closely related to FabZ. One of these, encoded by fabZ1 (SEQ ID NOs: 107 and 94) is somewhat more closely related to the bifunctional FabZ of E. faecalis than the other protein encoded by fabZ2. In one embodiment of this invention, a fabZ1 deletion mutant of L. plantarum PN0512 was designed, constructed and analyzed to show that the L. plantarum FabZ1 contributed to FabA-like activity required for unsaturated fatty acid biosynthesis.

A mutation in Lactobacillus in a gene present in single copy, whose product catalyzes isomerization of trans-2-decenoyl-Acyl Carrier Protein to cis-3-decenoyl-Acyl Carrier Protein will require exogenously added unsaturated fatty acids for growth. The results are shown in Example 4.

In one embodiment of this invention a Lactobacillus plantarum mutant lacking activity for isomerization of trans-2-decenoyl-Acyl Carrier Protein to cis-3-decenoyl-Acyl Carrier Protein is described as produced by the methods described in Example 4. The lack of activity for isomerization of trans-2-decenoyl-Acyl Carrier Protein to cis-3-decenoyl-Acyl Carrier Protein is indicated by auxotrophy for unsaturated fatty acids.

It is to be understood that the fabZ1 activity in Lactobacillus plantarum has been unknown so far, the said fabZ1 gene in this invention was characterized through gene disruption, auxotrophy of the mutant created by gene disruption and complementing the mutant strain for its auxotrophy. As a result the gene comprising nucleotide sequence (SEQ ID NO: 129) is designated fabZ1, and encoded protein FabZ1 with amino acid sequence (SEQ ID NO: 128), the said protein having activity for isomerization of trans-2-decenoyl-Acyl Carrier Protein to cis-3-decenoyl-Acyl Carrier Protein.

Two-Step Homologous Recombination Procedure for Constructing Markerless Gene Deletions

The method described in Example 4 may be applied for lactobacilli (bacteria of genus Lactobacillus) in general for construction of mutants or gene replacements. Any gene of fatty acid pathway may be disrupted or replaced by applying general teachings from this example. Other methods of preparing markerless deletions are described for other bacteria as well in literature. For example, method of generating markerless deletions in the Escherichia coli chromosome are described (Mizoguchi, H et. al., Bioscience, Biotechnology, and Biochemistry (2007), 71(12), 2905-2911). This method consists of two recombination events facilitated by λ Red recombinase. The first recombination replaces a target region with a marker cassette and the second then eliminates the marker cassette. The marker cassette includes an antibiotic resistant gene and a negative selection marker (Bacillus subtilis sacB) that makes E. coli sensitive to sucrose. Thus, a markerless deletion strain is successfully selected using its sucrose-resistant phenotype. To facilitate these recombination events, homologous sequences (left and right arms) flanking the target region are joined to both ends of the marker cassette or connected to each other by PCR. The marker cassette is then replaced with a fragment carrying a deletion by positively selecting for the loss of sacB gene.

In the present invention, the fabZ1 gene knockout construction used a two-step homologous recombination procedure to yield an unmarked gene deletion (Ferain et al., 1994, J. Bact. 176:596). Other genes of the unsaturated fatty acid biosynthetic pathway may also be used to alter the Ratio^{SFA/UFA} in the membrane of bacteria. The procedure in this invention utilized a shuttle vector pFP996pyrFΔerm (constructed in Example 3), derived from pFP996 which contains the pyrF sequence encoding orotidine-5′-phosphate decarboxylase from Lactobacillus plantarum PN0512 in place of the erythromycin coding region in pFP996. For selection purposes with pFP996pyrFEerm constructs, ampicillin was used for transformation in E. coli and growth on minimal medium in the absence of uracil was used in the L. plantarum PN0512ΔpyrF strain. The minimal medium consisted of constituents obtained from Sigma-Aldrich (St. Louis, Mo.): 0.1% Sodium Acetate, 1.92 g/L Yeast Synthetic Drop-Out Media Supplement without Uracil, 0.1% Tween-80, 0.03% L-Glutamic Acid Monosodium Salt Hydrate, 0.2% D(+)-Glucose Monohydrate, 6.7 g/L Yeast Nitrogen Base without Amino Acids.

Two segments of DNA, containing approximately 1200 bp of sequence upstream and downstream of the intended deletion, were cloned into the plasmid to provide the regions of homology for the two genetic cross-overs. Cells were grown for an extended number of generations to allow for the cross-over events to occur. The initial cross-over (single cross-over) integrated the plasmid into the chromosome by homologous recombination through one of the two homology regions on the plasmid. The second cross-over (double cross-over) event yielded either the wild type sequence or the intended gene deletion. A cross-over between the sequences that led to the initial integration event would yield the wild type sequence, while a cross-over between the other regions of homology would yield the desired deletion. The second cross-over event was screened for by a uracil auxotrophy. Single and double cross-over events were analyzed by PCR and DNA sequencing.

Homologous recombination in Lactobacillus plantarum is described by Hols et al. (Appl. Environ. Microbiol. 60:1401-1413 (1994))

Butanol Tolerance of Increased Membrane Saturated Fatty Acid Strain

A bacterial cell of the present invention modified for increased membrane saturated fatty acid composition has improved tolerance to butanol. The increased tolerance may be assessed by assaying growth in concentrations of butanol that are detrimental to growth of the unmodified or parental strain (prior to modification for increased membrane saturated fatty acid composition). Improved tolerance may be to butanol compounds including 1-butanol, isobutanol, 2-butanol or combinations thereof. The amount of tolerance improvement will vary depending on the inhibiting chemical and its concentration, growth conditions and the specific modified cell. For example, as shown in Example 2 herein, cells of L. plantarum having increased membrane saturated fatty acid composition had a growth yield in 2.5% to 3.0% (weight/volume) isobutanol that was between 1.23 and 1.92-fold higher than L. plantarum cells without increased membrane saturated fatty acid composition.

Butanol Biosynthetic Pathway

In the present invention, a modification conferring increased saturated fatty acid in the membrane is made in a bacterial cell that does not naturally produce butanol, but that has been engineered to express butanol biosynthetic pathway. Either modification may take place prior to the other.

The butanol biosynthetic pathway may be a 1-butanol, 2-butanol, or isobutanol biosynthetic pathway. Suitable biosynthetic pathways for production of butanol are known in the art, and certain suitable pathways are described herein. In some embodiments, the butanol biosynthetic pathway comprises at least one gene that is heterologous to the host cell. In some embodiments, the butanol biosynthetic pathway comprises more than one gene that is heterologous to the host cell. In some embodiments, the butanol biosynthetic pathway comprises heterologous genes encoding polypeptides corresponding to every step of a biosynthetic pathway.

Likewise, certain suitable proteins having the ability to catalyze indicated substrate to product conversions are described herein and other suitable proteins are provided in the art. For example, US Patent Application Publication Nos. US20080261230, US20090163376, US20100197519 and U.S. Provisional Patent Application No. 61/246,844, all incorporated herein by reference, describe acetohydroxy acid isomeroreductases; US Patent Application Publication No. 20100081154, incorporated by reference, describes dihydroxyacid dehydratases; alcohol dehydrogenases are described in US Published Patent Application US20090269823 and U.S. Provisional Application No. 61/290,636, both incorporated herein by reference.

Particularly suitable bacterial hosts for the production of butanol and modification for increased butanol tolerance include, but are not limited to, members of the genera Escherichia, Rhodococcus, Pseudomonas, Bacillus, Lactobacillus, and Enterococcus. Preferred hosts include: Escherichia coli, Pseudomonas putida, Lactobacillus plantarum, Enterococcus faecium, and Enterococcus faecalis.

1-Butanol Biosynthetic Pathway

A biosynthetic pathway for the production of 1-butanol is described by Donaldson et al. in co-pending and commonly owned U.S. Patent Application Publication No. US20080182308A1 incorporated herein by reference. This biosynthetic pathway comprises the following substrate to product conversions:

a) acetyl-CoA to acetoacetyl-CoA, as catalyzed for example by acetyl-CoA acetyltransferase encoded by the sequence provided as SEQ ID NO:1 or 3;

b) acetoacetyl-CoA to 3-hydroxybutyryl-CoA, as catalyzed for example by 3-hydroxybutyryl-CoA dehydrogenase encoded by the sequence provided as SEQ ID NO:5;

c) 3-hydroxybutyryl-CoA to crotonyl-CoA, as catalyzed for example by crotonase encoded by the sequence provided as SEQ ID NO:7;

d) crotonyl-CoA to butyryl-CoA, as catalyzed for example by butyryl-CoA dehydrogenase encoded by the sequence provided as SEQ ID NO:9 or 39;

e) butyryl-CoA to butyraldehyde, as catalyzed for example by butyraldehyde dehydrogenase encoded by the sequence provided as SEQ ID NO:11; and

f) butyraldehyde to 1-butanol, as catalyzed for example by 1-butanol dehydrogenase encoded by the sequence provided as SEQ ID NO:13 or 15.

The pathway requires no ATP and generates NAD⁺ and/or NADP⁺, thus, it balances with the central, metabolic routes that generate acetyl-CoA.

In some embodiments, the 1-butanol biosynthetic pathway comprises at least one gene, at least two genes, at least three genes, at least four genes, or at least five genes that is/are heterologous to the yeast cell.

2-Butanol Biosynthetic Pathway

Biosynthetic pathways for the production of 2-butanol are described by Donaldson et al. in co-pending and commonly owned U.S. Patent Application Publication Nos. US20070259410A1 and US 20070292927A1, both incorporated herein by reference. One 2-butanol biosynthetic pathway comprises the following substrate to product conversions:

a) pyruvate to alpha-acetolactate, as catalyzed for example by acetolactate synthase encoded by the sequence provided as SEQ ID NO:19;

b) alpha-acetolactate to acetoin, as catalyzed for example by acetolactate decarboxylase encoded by the sequence provided as SEQ ID NO:17;

c) acetoin to 2,3-butanediol, as catalyzed for example by butanediol dehydrogenase encoded by the sequence provided as SEQ ID NO:21;

d) 2,3-butanediol to 2-butanone, catalyzed for example by butanediol dehydratase encoded by sequence provided as SEQ ID NOs:23, 25, and 27; and

e) 2-butanone to 2-butanol, as catalyzed for example by 2-butanol dehydrogenase encoded by the sequence provided as SEQ ID NO:29.

In some embodiments, the 2-butanol biosynthetic pathway comprises at least one gene, at least two genes, at least three genes, or at least four genes that is/are heterologous to the yeast cell.

Isobutanol Biosynthetic Pathway

Biosynthetic pathways for the production of isobutanol are described by Maggio-Hall et al. in co-pending and commonly owned U.S. Patent Application Publication No. US20070092957 A1, incorporated herein by reference. One isobutanol biosynthetic pathway comprises the following substrate to product conversions:

a) pyruvate to acetolactate, as catalyzed for example by acetolactate synthase encoded by the gene given as SEQ ID NO:19;

b) acetolactate to 2,3-dihydroxyisovalerate, as catalyzed for example by acetohydroxy acid isomeroreductase encoded by the gene given as SEQ ID NO:31 or 41;

c) 2,3-dihydroxyisovalerate to α-ketoisovalerate, as catalyzed for example by acetohydroxy acid dehydratase encoded by the gene given as SEQ ID NO:33;

d) α-ketoisovalerate to isobutyraldehyde, as catalyzed for example by a branched-chain keto acid decarboxylase encoded by the gene given as SEQ ID NO:35; and

e) isobutyraldehyde to isobutanol, as catalyzed for example by a branched-chain alcohol dehydrogenase encoded by the gene given as SEQ ID NO:37.

In some embodiments, the isobutanol biosynthetic pathway comprises at least one gene, at least two genes, at least three genes, or at least four genes that is/are heterologous to the yeast cell.

Construction of Bacterial Strains for Butanol Production

Any bacterial strain that is modified for butanol tolerance as described herein is additionally genetically modified (before or after modification to tolerance) to incorporate a butanol biosynthetic pathway by methods well known to one skilled in the art. The DNA sequences and their protein products comprising enzyme activities described above, or corresponding orthologs may be identified and obtained by commonly used methods well known to one skilled in the art, are introduced into a bacterial host. Representative coding and amino acid sequences for pathway enzymes that may be used are given in Tables 1, 2, and 3, with SEQ ID NOs:1-42. Typically BLAST (described above) searching of publicly available databases with the provided amino acid sequences is used to identify homologs and their encoding sequences that may be used in butanol biosynthetic pathways in the present cells. For example, proteins having amino acid sequence identities of at least about 70-75%, 75%-80%, 80-85%, 85%-90%, 90%-95% or 98% sequence identity to any of the proteins in Tables 1, 2, or 3 and having the noted activities may be identified. Identities are based on the Clustal W method of alignment using the default parameters of GAP PENALTY=10, GAP LENGTH PENALTY=0.1, and Gonnet 250 series of protein weight matrix. In addition to using protein or coding region sequence and bioinformatics methods to identify additional homologs, the sequences described herein or those recited in the art may be used to experimentally identify other homologs in nature as described above for fatty acid cis-trans isomerase.

Methods described in co-pending and commonly owned U.S. Patent Application Publication Nos. US20080182308A1, US20070259410A1, US 20070292927A1, and US20070092957 A1 may be used to engineer bacteria for expression of a butanol biosynthetic pathway. Vectors or plasmids useful for the transformation of a variety of host cells are common and commercially available from companies such as EPICENTRE® (Madison, Wis.), Invitrogen Corp. (Carlsbad, Calif.), Stratagene (La Jolla, Calif.), and New England Biolabs, Inc. (Beverly, Mass.). Typically, the vector or plasmid contains sequences regulating transcription and translation of the relevant gene, a selectable marker, and sequences allowing extrachromosomal autonomous replication or chromosomal integration. Suitable vectors comprise a region 5′ upstream of the gene which harbors transcriptional initiation controls and a region 3′ downstream of the DNA fragment which controls transcriptional termination. Both control regions may be derived from genes homologous to the transformed host cell, although it is to be understood that such control regions may also be derived from genes that are exogenous to the specific species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention including, but not limited to, lac, ara, tet, trp, IPL, IPR, T7, tac, and trc (useful for expression in Escherichia coli and Pseudomonas); the amy, apr, npr promoters and various phage promoters useful for expression in Bacillus subtilis, and Bacillus licheniformis; nisA (useful for expression Gram-positive bacteria, Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)). Termination control regions may also be derived from various genes native to the preferred hosts. Optionally, a termination site may be unnecessary, however, it is most preferred if included.

Certain vectors are capable of replicating in a broad range of host bacteria and can be transferred by conjugation. The complete and annotated sequence of pRK404 and three related vectors-pRK437, pRK442, and pRK442(H) are available. These derivatives have proven to be valuable tools for genetic manipulation in Gram-negative bacteria (Scott et al., Plasmid 50(1):74-79 (2003)). Several derivatives of broad-host-range Inc P4 plasmid RSF1010 are also available with promoters that can function in a range of Gram-negative bacteria. Plasmid pAYC36 and pAYC37, have active promoters along with multiple cloning sites to allow for the heterologous gene expression in Gram-negative bacteria.

Chromosomal gene replacement tools are also widely available. For example, a thermosensitive variant of the broad-host-range replicon pWV101 has been modified to construct a plasmid pVE6002 which can be used to create gene replacement in a range of Gram-positive bacteria (Maguin et al., J. Bacteriol. 174(17):5633-5638 (1992)).

Other suitable modifications are known in the art. For example, U.S. Provisional Patent Application No. 61/246,717, incorporated herein by reference, discloses modifications in lactic acid bacterial cells. Modifications to a host cell that provide for increased carbon flux through an Entner-Doudoroff Pathway or reducing equivalents balance as described in US Patent Application Publication No. 20100120105 (incorporated herein by reference). Other modifications include modifications in an endogenous polynucleotide encoding a polypeptide having dual-role hexokinase activity, described in U.S. Provisional Application No. 61/290,639, integration of at least one polynucleotide encoding a polypeptide that catalyzes a step in a pyruvate-utilizing biosynthetic pathway described in U.S. Provisional Application No. 61/380,563 (both referenced provisional applications are incorporated herein by reference in their entirety).

Additionally, host cells comprising at least one deletion, mutation, and/or substitution in an endogenous gene encoding a polypeptide affecting Fe—S cluster biosynthesis are described in U.S. Provisional Patent Application No. 61/305,333 (incorporated herein by reference), and host cells comprising a heterologous polynucleotide encoding a polypeptide with phosphoketolase activity and host cells comprising a heterologous polynucleotide encoding a polypeptide with phosphotransacetylase activity are described in U.S. Provisional Patent Application No. 61/356,379.

Construction of Lactobacillus Strains for Butanol Production

The Lactobacillus genus belongs to the Lactobacillaceae family and many plasmids and vectors used in the transformation of Bacillus subtilis and Streptococcus may be used for Lactobacillus. Non-limiting examples of suitable vectors include pAMβ1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol. 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Several plasmids from Lactobacillus plantarum have also been reported (van Kranenburg R, Golic N, Bongers R, Leer R J, de Vos W M, Siezen R J, Kleerebezem M. Appl. Environ. Microbiol. 2005 March; 71(3): 1223-1230), which may be used for transformation.

Initiation control regions or promoters, which are useful to drive expression of the relevant pathway coding regions in the desired Lactobacillus host cell, may be obtained from Lactobacillus or other lactic acid bacteria, or other Gram-positive organisms. A non-limiting example is the nisA promoter from Lactococcus. Termination control regions may also be derived from various genes native to the preferred hosts or related bacteria.

The various genes for a butanol biosynthetic pathway may be assembled into any suitable vector, such as those described above. The codons can be optimized for expression based on the codon index deduced from the genome sequences of the host strain, such as for Lactobacillus plantarum or Lactobacillus arizonensis. The plasmids may be introduced into the host cell using methods known in the art, such as electroporation, as described in any one of the following references: Cruz-Rodz et al. (Molecular Genetics and Genomics 224:1252-154 (1990)), Bringel and Hubert (Appl. Microbiol. Biotechnol. 33: 664-670 (1990)), and Teresa Alegre, Rodriguez and Mesas (FEMS Microbiology letters 241:73-77 (2004)). Plasmids can also be introduced to Lactobacillus plantatrum by conjugation (Shrago, Chassy and Dobrogosz Appl. Environ. Micro. 52: 574-576 (1986)). The butanol biosynthetic pathway genes can also be integrated into the chromosome of Lactobacillus using integration vectors (Hols et al. Appl. Environ. Micro. 60:1401-1403 (1990); Jang et al. Micro. Lett. 24:191-195 (2003)).

Fermentation of Butanol Tolerant Bacteria for Butanol Production

The present cells with increased membrane saturated fatty acid composition and having a butanol biosynthesis pathway may be used for fermentation production of butanol.

Fermentation media for the production of butanol must contain suitable carbon substrates. Suitable substrates may include but are not limited to monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Sucrose may be obtained from feedstocks such as sugar cane, sugar beets, cassava, and sweet sorghum. Glucose and dextrose may be obtained through saccharification of starch based feedstocks including grains such as corn, wheat, rye, barley, and oats.

In addition, fermentable sugars may be obtained from cellulosic and lignocellulosic biomass through processes of pretreatment and saccharification, as described, for example, in US Patent Application Publication US20070031918A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure and other biological waste.

Although it is contemplated that all of the above mentioned carbon substrates and mixtures thereof are suitable in the present invention, preferred carbon substrates are glucose, fructose, and sucrose.

In addition to an appropriate carbon source, fermentation media must contain suitable minerals, salts, cofactors, buffers and other components, known to those skilled in the art, suitable for the growth of the cultures and promotion of the enzymatic pathway necessary for butanol production.

Typically cells are grown at a temperature in the range of about 25° C. to about 40° C. in an appropriate medium. Suitable growth media are common commercially prepared media such as Bacto Lactobacilli MRS broth or Agar (Difco), Luria Bertani (LB) broth, Sabouraud Dextrose (SD) broth or Yeast Medium (YM) broth. Other defined or synthetic growth media may also be used, and the appropriate medium for growth of the particular bacterial strain will be known by one skilled in the art of microbiology or fermentation science. The use of agents known to modulate catabolite repression directly or indirectly, e.g., cyclic adenosine 2′:3′-monophosphate, may also be incorporated into the fermentation medium.

Suitable pH ranges for the fermentation are between pH 5.0 to pH 9.0, where pH 6.0 to pH 8.0 is preferred as the initial condition.

Fermentations may be performed under aerobic or anaerobic conditions, where anaerobic or microaerobic conditions are preferred.

Butanol may be produced using a batch method of fermentation. A classical batch fermentation is a closed system where the composition of the medium is set at the beginning of the fermentation and not subject to artificial alterations during the fermentation. A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the media. Batch and fed-batch fermentations are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference.

Butanol may also be produced using continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology and a variety of methods are detailed by Brock, supra.

It is contemplated that the production of butanol may be practiced using either batch, fed-batch or continuous processes and that any known mode of fermentation would be suitable. Additionally, it is contemplated that cells may be immobilized on a substrate as whole cell catalysts and subjected to fermentation conditions for butanol production.

Methods for Butanol Isolation from the Fermentation Medium

Bioproduced butanol may be isolated from the fermentation medium using methods known in the art for ABE fermentations (see for example, Durre, Appl. Microbiol. Biotechnol. 49:639-648 (1998), Groot et al., Process. Biochem. 27:61-75 (1992), and references therein). For example, solids may be removed from the fermentation medium by centrifugation, filtration, decantation, or the like. Then, the butanol may be isolated from the fermentation medium using methods such as distillation, azeotropic distillation, liquid-liquid extraction, adsorption, gas stripping, membrane evaporation, or pervaporation.

### EXAMPLES

The following abbreviations will be used for the interpretation of the specification and the claims.

The meaning of abbreviations used is as follows: “kb” means kilobase(s), “min” means minute(s), “h” or “hr” means hour(s), “sec’ means second(s), “d” means day(s), “nl” means nanoliter(s), “μl” means microliter(s), “ml” means milliliter(s), “L” means liter(s), “nm” means nanometer(s), “mm” means millimeter(s), “cm” means centimeter(s), “μm” means micrometer(s), “μM” means micromolar, “mM” means millimolar, “M” means molar, “mmol” means millimole(s), “μmole” means micromole(s), “g” means gram(s), “ng” means nanogram(s), “μg” means microgram(s), “mg” means milligram(s), “rpm” means revolutions per minute, “w/v” means weight/volume, “Cm” means chloramphenicol, “OD” means optical density, and “OD₆₀₀” means optical density measured at a wavelength of 600 nm.

The present invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various uses and conditions. For example, a variety of bacterial media are known in the literature that may be adapted for fatty acid feeding experiments. Saturated fatty acids may be fed by incorporation in culture media in a concentration range of 10-500 mg per liter culture medium. The lipid fatty acid extraction methods, FAME analysis are methods broadly applicable to all bacterial species. Growth may be analyzed by measuring optical density, cell numbers, cell viability or survival over time and other methods using well known metrics in the art for bacterial growth.

All restriction enzymes, DNA modifying enzymes and Phusion High-Fidelity PCR Master Mix were purchased from NEB Inc. (Ipswich, Ma). DNA fragments were purified using Qiaquick PCR Purification Kit (Qiagen Inc., Valencia, Calif.). Plasmid DNA was prepared with QIAprep Spin Miniprep Kit (Qiagen Inc., Valencia, Calif.). Oligonucleotides were synthesized by Invitrogen Corp (Carlsbad, Calif.). L. plantarum strain PN0512 genomic DNA was prepared with MasterPure DNA Purification Kit (Epicentre, Madison, Wis.).

### General Methods

A semi-synthetic growth medium namely LAB medium, was used. pH7 or pH6, with bovine serum albumin (BSA) used as a carrier. The composition of this medium is:

0.01 M Ammonium Sulfate

0.005 M Potassium Phosphate, pH 7.0 OR pH 6.0

0.05 M MOPS, pH 7.0 OR 0.05M MES, pH 6.0

1% S10 Metal Mix

0.01 M Glucose

0.2% Yeast Extract

0.01% Casamino Acids

5 g/l BSA

The composition of S10 Metal Mix is:

200 mM MgCl₂

70 mM CaCl₂

5 mM MnCl₂

0.1 mM FeCl₃

0.1 mM ZnCl₂

0.2 mM Thiamine Hydrochloride

172 μM CuSO₄

253 μM CoCl₂

242 μM Na₂MoO₄

All ingredients for medium were purchased from Sigma Chemical Company (St. Louis, Mo.) except yeast extract and casamino acids, which were purchased from Beckton, Dickinson and Co (Sparks, Md.). Free fatty acids, added to a final concentration of 50 mg/ml from 1% ethanol stock solutions (stored at −20° C.), were purchased from Sigma Chemical Company (St Louis, Mo.), Isobutanol was purchased from Sigma Chemical Company (St. Louis, Mo.).

A working stock of Lactobacillus plantarum PN0512 (ATCC # PTA-7727) was prepared to use as a consistent source of inoculum. Cultures were grown in MRS medium (Acumedia Manufacturers, Inc. Lansing, Mich.) at 30° C. overnight. Glycerol was added to a final concentration of 12.5% and aliquots were frozen at −80° C. One aliquot was thawed at room temperature and used to inoculate all tubes in an experiment and then discarded.

Growth Analysis

For growth yield experiments, 5 ml of medium with test fatty acids (10-500 mg per liter) and varying concentrations of isobutanol in 15 ml screw cap tubes was inoculated with 12.5 μl of the working stock giving an initial OD₆₀₀ of 0.012. The caps were tightly sealed and incubated at 30° C. on a roller drum for 20 to 26 hours, at which time 1.0 ml was removed and OD₆₀₀ was measured with a blank of medium amended with the fatty acid. All solvent concentrations are reported as % (w/v).

Lipid Extraction

The membrane lipids were extracted by modified Bligh and Dyer protocol (Can. J. Biochem. Physiol. (1959) 37:911-17). The cell pellet prepared as described above was suspended in a mixture of 0.5 ml CHCl₃ and 1 ml CH₃OH, and transferred to a 13×100 mm tube with a screw top cap. The cap was screwed on about ¾ of the way (i.e., not tight), and the tube was incubated at 40° C. for 30 min. The tube was cooled and an additional 0.5 ml CHCl₃ and 1 ml H₂O were added the mixture. This results in the formation of two phases. The two phases were equilibrated by vortexing. The two phases were allowed to separate; then the lower CHCl₃ layer was removed and transferred to another 13×100 mm tube with a screw top cap. With the cap removed, the CHCl₃ was evaporated under a stream of N₂. Methyl esters of the fatty acids in the residue were then formed using one of the following procedures.

Formation of Fatty Acid Methyl Esters by Transesterification Using CH₃ONa in CH₃OH

1 ml freshly made 1.0 M CH₃ONa in CH₃OH was added to the tubes containing lipid samples extracted by the Bligh and Dyer method as described above. The caps were placed on tubes, screwed on about ¾ of the way (i.e., not tight), then the tubes were heated at 60° C. for 30 minutes. The mixture was chilled in ice bath and 1 ml of 1.0 N HCl was added to the solution in the tubes. The pH of the resulting solution was checked with pH paper to make sure a pH of 7 or lower had been reached. 0.5 ml hexane was added into the test tube and mixed well by vortexing. The tubes were allowed to sit for a few minutes until two phases formed. The top hexane layer was removed and placed in a separate tube for storage until analysis, which was done by GC/FID and/or GC/MS. 2 μl of the hexane layer was injected into an Agilent GC (model 6890)/MS (model 5973). For routine samples a Supelco Equity-1 column (15 m×0.25 mm×0.25 μm film thickness; catalog #28045-U) was used with an FID detector (GC/FID). When an unknown peak needed to be identified, the same column was used with an Agilent MSD detector (GC/MS). When samples requiring difficult separations that were impossible to achieve on a 15 m column were analyzed (e.g., the separation of oleic from elaidic acid), a Supelco S-2380 column (100 m×0.25 mm×0.25 μm film thickness; catalog #24317) was used.

Preparation of Samples for FAME (Fatty Acid Methyl Ester) Analysis.

For preparation of samples for FAME analysis, the working stock was used to inoculate 40 ml of medium containing free fatty acids and the cultures were grown overnight. The cell pellet was harvested by centrifugation and was washed twice with phosphate buffered saline (PBS, Bio-Rad Laboratories, Hercules, Calif.) and 5 g/l BSA, then two more times with PBS. Cell pellets were stored at −80° C. until analyzed by FAME using a transesterification protocol, which quantifies fatty acids that have been incorporated in membrane lipids, but not free fatty acids associated with the cell membrane. The FAME analysis is described by Christie (1993) (In Advances in Lipid Methodology—Two, pp. 69-111, Ed. W. W. Christie, Oily Press, Dundee).

### Example 1

### Incorporation of Fed Saturated Fatty Acid into Membrane Lipids of L. plantarum Strain PN0512

The purpose of this example is to demonstrate that levels of saturated fatty acids in membrane lipids can be increased by feeding saturated fatty acids in the medium.

Cultures of Lactobacillus plantarum strain PN0512 were grown in media containing stearic acid along with control cultures (with no added fatty acids to the media), as described in General Methods. The membrane composition was analyzed by FAME analysis as described in General Methods as well. The results of FAME analyses shown in Table 6 indicate that stearic acid (C18:0), when added to the growth medium of a culture of strain PN0512, was incorporated into the cell membrane thereby resulting in a substantial increase of the amount of stearic fatty acid in the cell membrane.

TABLE 6
Levels (molar %) of saturated fatty acid (C18:0, cyc-C19:0)
in membrane lipids was increased by feeding stearic acid (C18:0, a
saturated fatty acid) in the media of L. plantarum strain PN0512 cultures.

| | | Stearic Acid | |
|---|---|---|---|
| | Control | (C18:0) | Membrane Fatty Acid |
| | (fatty acid not fed) | (fatty acid fed) | Effect |
| Membrane | Membrane Fatty Acid Content | | stearic acid fed)/ |
| Fatty Acid | molar % | | stearic acid not fed) |
| C16:0 | 27.1 | 14.1 | 0.52 |
| C16:1 | 7.7 | 10.0 | 1.30 |
| C18:0 | 0.5 | 16.6 | 33.2 |
| C18:1, cis | 44.3 | 34.4 | 0.78 |
| cyc-C19:0 | 20.4 | 25.0 | 1.23 |

The molar % of stearic acid, a saturated fatty acid of 18-carbon length, in the cell membrane increased more than 30-fold with stearic acid feeding. The molar % of other constituent fatty acids also changed with stearic acid feeding. Nonetheless, the molar ratio of saturated fatty acids (C16:0 and C18:0) to unsaturated fatty acids (C16:1 and C18:1, cis) increased from 0.53 to 0.69 (a 16% increase) with stearic acid feeding. See calculations below:

Ratio^{SFA/UFA}=(Molar % C16:0+Molar % C18:0)÷(Molar % C16:1+Molar % C18:1)

Thus, these growth conditions yielded cell cultures with substantially different cell membranes. Cell cultures thus obtained with substantially different cell membranes were used in the forthcoming Example 2 to determine the effect of elevated saturated fatty acids in the membrane lipids on butanol tolerance.

### Example 2

### Improved Tolerance to Isobutanol with Increased Saturated Fatty Acids in the Cell Membrane

As shown in Example 1, feeding L. plantarum strain PN0512 cells stearic acid resulted in membranes containing increased saturated fatty acids ratios. Growth of L. plantarum cultures in the media described in General Methods and containing varying concentrations of isobutanol was measured and compared with the cultures fed (supplemented with) stearic acid at a final concentration of 50 mg per liter of medium. Cultures were prepared as described in General Methods. Table 7 shows the data as an average of two independent experiments comparing the growth yield of stearic acid fed and unfed cultures of L. plantarum strain PN0512 after 25 hours of incubation at 30° C. in various concentrations of isobutanol.

TABLE 7
Growth yield data (measured as optical density, OD₆₀₀) for stearic
acid fed L. plantarum strain PN0512 in the presence of isobutanol.

| | | | OD₆₀₀ Ratio |
|---|---|---|---|
| [Isobutanol] % | OD₆₀₀ unfed | OD₆₀₀ fed | OD₆₀₀fed/OD₆₀₀unfed |
| w/v | control | Stearic | control |
| 0 | 1.337 | 1.452 | 1.08 |
| 2.3 | 0.727 | 0.860 | 1.18 |
| 2.5 | 0.341 | 0.422 | 1.23 |
| 2.7 | 0.131 | 0.206 | 1.57^{b} |
| 2.9 | 0.051 | 0.098 | 1.92 |

^{b}57% higher growth yield or growth yield increased by a factor of 1.57.

These results show that at all tested concentrations of isobutanol, the growth yield of the stearic acid fed cultures was greater than the growth yield of the control cultures. For example, for cultures grown in 2.7% w/v isobutanol, the growth yield was 57% higher in the stearic acid fed cultures than in the control cultures. These results are consistent with greater isobutanol tolerance of the culture with a high levels of saturated fatty acids in the membrane.

### Example 3

### Selectable-Counterselectable Marker System for Gene Disruptions in L. plantarum Strain PN0512

The term pyrF refers to a gene that encodes a pyrimidine biosynthetic enzyme having orotidine-5′-monophosphate (OMP) decarboxylase activity (EC 4.1.1.23). The pyrF gene of L. plantarum strain PN0512, was engineered as a selectable-counterselectable marker. First, the naturally occurring pyrF gene was disrupted in the strain PN0512. Next, an E. coli shuttle vector containing the pyrF gene was constructed to complement the uracil auxotrophy of the deletion strain.

Construction of a L. plantarum ΔpyrF strain. A putative pyrimidine biosynthesis operon annotation of the L. plantarum strain WCFS1 genome (NCBI reference sequence: NC_{—}004567.1) was used to retrieve the nucleotide sequence. The putative pyr operon, located between bases (nucleotides) 2393220 and 2407835, was used to design PCR primers for amplification of a putative pyrF and surrounding genes in the L. plantarum strain PN0512. The upstream gene, pyrD, was fused to the downstream genes pyrE and oroP by PCR using primers N378 (SEQ ID NO:43) and N394-N396 (SEQ ID NOs: 44-46). The PCR product was cloned into a plasmid pCR4Blunt-TOPO (Invitrogen Cat. No. K2835). Three independent clones were sequenced using primers N374 (SEQ ID NO: 47), N375 (SEQ ID NO: 48), N378-N381 (SEQ ID NO: 43, 49-51 respectively). One clone was digested with EcoRI and HindIII and the resultant 2.7 kb pyrDEoroP fragment was ligated into pFP996 cut with the same enzymes.

pFP996 is a shuttle plasmid (also referred as shuttle vector) that can replicate in both E. coli and gram-positive bacteria. It contains the E. coli origin of replication (nucleotides 2628 to 5323) from pBR322 (Cold Spring Harb. Symp. Quant. Biol. 43 Pt 1, 77-90. 1979) and gram positive origin of replication (nucleotides 43-2627) from pE194. pE194 is a small plasmid isolated originally from a gram positive bacterium, Staphylococcus aureus (Horinouchi and Weisblum J. Bacteriol. (1982) 150(2):804-814). The pFP996 multiple cloning site (nucleotides 1 to 60) contains restriction sites for EcoRI, BgIII, XhoI, XmaI, ClaI, KpnI, HindIII, and BsrGI. In pFP996, there are two antibiotic resistance markers; one is for resistance to ampicillin and the other for resistance to erythromycin.

The ligation reaction was transformed into E. coli TOP10 cells (Invitrogen Cat. No. K4575) using manufacturer's protocol and ampicillin selection was used (100 μg/ml) to select for transformants on LB medium. After confirmation by PCR using primers N378 (SEQ ID NO: 43) and N379 (SEQ ID NO: 49) and restriction digestion (EcoRI/BamHI), the plasmid was introduced into L. plantarum strain PN0512 by electroporation as described by Aukrust et al. (pp. 201-208, Methods in Molecular Biology, Vol. 47: Electroporation Protocols for Microorganisms, J. A. Nickoloff, Ed., Humana Press Inc., Totowa N.J.). Transformants were selected on MRS medium (Accumedia, Neogen Corporation, Lansing, Mich.) containing 1 μg/ml erythromycin. After confirming successful introduction of the plasmid into the strain (by colony PCR using primers N374 (SEQ. ID NO: 47) and N379 (SEQ ID NO: 49), the strain was cultured in liquid MRS medium at 37° C. for 50 generations with one subculture per day. Culture was then plated on MRS medium containing 1 μg/ml erythromycin to select for cells that had integrated the vector. Successful integration at the pyr locus by single cross-over was confirmed by PCR (primers N435-N438 described by SEQ ID NOs. 52-55, respectively). Several integrants were obtained, all containing integration via recombination downstream of pyrF. In order to select for a second cross-over event that removed vector sequences and the wild-type pyrF gene, leaving behind the non-polar deletion of pyrF. the cells were plated at 37° C. on yeast synthetic complete medium (Methods in Yeast Genetics, Amberg, Burke and Strathern, eds., Cold Spring Harbor Laboratory Press, 2005) that had been supplemented with Tween 80 (0.1%), acetate (0.1%), glutamate (0.03%), uracil (0.05%) and 100 μg/ml 5-fluoroorotic acid. One out of ten L. plantarum colonies obtained on the 5-FOA plates was erythromycin sensitive, indicating loss of the pFP996 vector due to double cross over recombination and carried the pyrF deletion (as assessed by PCR, primers N376-N377 (SEQ ID NO: 56 and SEQ ID NO: 57), N435-N436 (SEQ ID NO: 52 and SEQ ID NO: 53) and N437-N438 (SEQ ID NO: 54 and SEQ ID NO: 55), and were uracil auxotrophs (assessed by plating on amended synthetic complete medium without uracil). One such strain was retained and named BP15.

Construction of an E. coli-L. plantarum Shuttle Vector Carrying a pyrF Selectable Marker.

The pyrF gene was amplified from PN0512 genomic DNA using primers N452-N453 (SEQ ID NO: 58 and SEQ ID NO: 59) The erm promoter was amplified from pFP996 using primers N450-N451 (SEQ ID NO: 60 and SEQ ID NO: 61). These two PCR products were fused by an additional round of PCR. The resulting PCR product was cloned into pCR4Blunt-TOPO (Invitrogen Cat. No. K2835) according to the manufacturer's instructions. Three resulting clones were sequenced. One was digested with SacI and NsiI to release the 0.77 kb erm promoter-pyrF fragment. This was cloned into pFP996 restricted with SacI and NsiI. This plasmid modification removes most of the erythromycin resistance (erm) gene coding region and places the pyrF gene (minus the first codon) in frame after the fifth codon of erm. The ligation reaction was transformed into E. coli TOP10 cells (Invitrogen Cat. No. K4575) according to the manufacturer's instructions. Introduction of the pyrF gene into the vector was confirmed by PCR using primers N377 (SEQ ID NO:57) and N452 (SEQ ID NO: 58). The new vector named pFP996pyrFΔerm is an E. coli-L. plantarum shuttle vector. pFP996pyrFΔerm, was transformed into the L. plantarum PN0512 ΔpyrF strain. Cells were washed twice with sterile solution of 1× yeast nitrogen base (Amresco Cat. No. J386) and were plated on amended synthetic complete medium without uracil. After two days, transformant colonies were observed, confirming the presence of a functional plasmid-borne pyrF marker.

### Example 4

### Construction of a fabZ1 Deletion in L. plantarum PN0512ΔpyrF

If, as predicted, unsaturated fatty acid biosynthesis in L. plantarum requires the fabZ1 gene product, then the fabZ1 mutant strain should be unable to grow in the absence of an external source of unsaturated fatty acids. Thus, L. plantarum PN0512ΔpyrF was transformed with the pFP996pyrFΔerm-fabZ1 arms construct by electroporation. pFP996pyrFΔerm-fabZ1 arms is derived from pFP996pyrFΔerm by incorporating homologous arms for the purpose of constructing a chromosomal fabZ1 deletion in Lactobacillus plantarum PN0512ΔpyrF.

Construction of pFP996pyrFΔerm-fabZ1 arms: The homologous arms for were amplified from L. plantarum strain PN0512 genomic DNA. The fabZ1 upstream homologous arm was amplified using oligonucleotides oBP15 (SEQ ID NO:62) containing a BgIII restriction site and oBP16 (SEQ ID NO:63) containing an Xmal restriction site. The fabZ1 downstream homologous arm was amplified using oligonucleotides oBP17 (SEQ ID NO:64) containing an Xmal restriction site and oBP18 (SEQ ID NO:65) containing a KpnI restriction site. The fabZ1 upstream homologous arm was digested with BgIII and XmaI and the fabZ1 downstream homologous arm was digested with XmaI and KpnI. The two homologous arms were ligated with T4 DNA Ligase into the corresponding restriction sites of pFP996pyrFΔerm after digestion with the appropriate restriction enzymes to create vector pFP996pyrFΔerm-fabZ1 arms.

Preparation of Lactobacillus plantarum PN0512ΔpyrF electrocompetent cells: 5 ml of Lactobacilli MRS medium (Accumedia, Neogen Corporation, Lansing, Mich.) containing 1% glycine (Sigma-Aldrich, St. Louis, Mo.) was inoculated with PN0512ΔpyrF cells and grown overnight at 30° C. 100 ml MRS medium with 1% glycine was inoculated with overnight culture to an OD₆₀₀ of 0.1 and grown to an OD₆₀₀ of 0.7 at 30° C. Cells were harvested at 3700×g for 8 min at 4° C., washed with 100 ml cold 1 mM MgCl₂ (Sigma-Aldrich, St. Louis, Mo.), centrifuged at 3700×g for 8 min at 4° C., washed with 100 ml cold 30% PEG-1000 (Sigma-Aldrich, St. Louis, Mo.), recentrifuged at 3700×g for 20 min at 4° C., then resuspended in 1 ml cold 30% PEG-1000.

Electrotransformation of Lactobacillus plantarum PN0512ΔpyrF and screening for single crossovers integrants: 60 μl of electrocompetent cells were mixed with approximately 100 ng of plasmid DNA (pFP996pyrFΔerm-fabZ1 arms) in a cold 1 mm gap electroporation cuvette and electroporated in a BioRad Gene Pulser (Hercules, Calif.) at 1.7 kV, 25 pF, and 400Ω. Cells were resuspended in 1 ml MRS medium containing 500 mM sucrose (Sigma-Aldrich, St. Louis, Mo.) and 100 mM MgCl₂, incubated at 30° C. for 2 hrs, plated on minimal medium plates without uracil, then placed in an anaerobic box containing a Pack-Anaero sachet (Mitsubishi Gas Chemical Co., Tokyo, Japan) and incubated at 30° C. Transformants were grown at 30° C. in minimal medium without uracil for approximately 10 generations in an anaerobic box containing a Pack-Anaero sachet, followed by growth at 42° C. for approximately 20 generations by serial inoculations in minimal medium without uracil in an anaerobic box containing a Pack-Anaero sachet. Cultures were plated on minimal medium without uracil and isolates were screened by colony PCR for a single cross-over with chromosomal specific oligonucleotide oBP45 (SEQ ID NO:67) and plasmid specific oligonucleotide oBP42 (SEQ ID NO:66). Colony PCR was carried out using standard conditions with a hot-start enzyme mix (Invitrogen Platinum PCR Supermix HiFi, Carlsbad, Calif.) with an initial hold of 5 minutes at 94° C.

Screening for double crossover recombinants: Single cross-over integrants were grown at 37° C. for approximately 40 generations by serial inoculations under non-selective conditions in Lactobacilli MRS medium. Cultures were plated on MRS medium and isolates were patched to MRS plates, grown at 37° C., and then patched onto minimal medium plates without uracil. Uracil auxotroph isolates were screened by colony PCR for the presence of a wild-type or deletion second cross-over using chromosomal specific oligonucleotides oBP45 (SEQ ID NO: 67) and oBP52 (SEQ ID NO: 68). A wild-type sequence yielded a 3000 bp product and a deletion sequence yielded a 2580 bp product. The deletions were confirmed by sequencing the PCR product and absence of plasmid was tested by colony PCR. One fabZ1 deletion isolate, named BP63, was saved for analysis. In strain BP63 (L. plantarum PN0512ΔpyrFΔfabZ1) amino acids 1-140 of 147 were deleted from L. plantarum PN0512 fabZ1 gene (SEQ ID No: 128 and SEQ ID No: 129).

### Example 5

### Unsaturated Fatty Acid Auxotrophy of the fabZ1 Deletion Strain and Isobutanol Stimulated Growth

Strain BP63 (ΔfabZ1, described in Example 4) and the parental strain BP15 (fabZ1⁺, described in Example 3) were grown in semi-synthetic LAB medium, pH6, with 75 μg/ml uracil and 2.5 μg/mL hematin in the presence and absence of an unsaturated fatty acid, oleic acid. Cultures were prepared as described in General Methods. Table 8 displays the growth yield of cultures of BP15 (fabZ1⁺) and BP63 (ΔfabZ1) after 24 hours of incubation at 30° C. with different amounts of oleic acid (C18:1).

TABLE 8
Growth of the fabZ1 deletion strain BP63 (ΔfabZ1) and parental
strain BP15 (fabZ1⁺) in the presence and absence of oleic acid.

OD₆₀₀

| Oleic acid | BP15 | BP63 |
|---|---|---|
| mg/liter | (fabZ1⁺) | (ΔfabZ1) |
| 0 | 0.9237 | 0.0207 |
| 1.5 | 0.9449 | 0.0091 |
| 3 | 0.8375 | 0.0081 |
| 6 | 0.9459 | 0.0084 |
| 12 | 0.9681 | 0.0234 |
| 25 | 1.0069 | 0.6943 |
| 100 | 1.0915 | 1.1452 |
| 200 | 1.3187 | 1.3725 |

There was essentially no growth of the BP63 in the absence of oleic acid or at low concentrations of oleic acid up to 12 mg/liter. With 100 or 200 mg/liter of oleic acid the growth of BP63 was equivalent to that of the fabZ1⁺ control strain, BP15. These results are consistent with a unsaturated fatty acid auxotropy conferred by the fabZ1 mutation. Thus, we conclude that fabZ1 in L. plantarum has the same function as FabN in E. faecalis (Wang, H. and Cronan, J. E. 2004. Functional replacement of the FabA and FabB proteins of Escherichia coli fatty acid synthesis by Enterococcus faecalis FabZ and FabF homologs. J. Biol. Chem. 279, 34489-95). To further test the range of fatty acid supplements that support growth of the fabZ1 mutant, several other fatty acids were supplied at 80 mg/L to the semi-synthetic LAB medium as described above. BP63 and the parental control BP15 were inoculated from the working stocks. After overnight incubation, the OD₆₀₀ was measured. The growth of BP15 was not inhibited by any of the fatty acids tested. The Table 9 below summarizes the results for the fabZ1 mutant strain, BP63.

TABLE 9
Effect of a variety of fatty acids on the growth on L. plantarum
PN0512ΔpyrFΔfabZ1.

| | | Supports growth |
|---|---|---|
| Fatty acid name | Code | of BP63 |
| Myristic | C14:0 (saturated) | no |
| Palmitic | C16:0 (saturated) | no |
| Stearic | C18:0 (saturated) | no |
| Palmitoleic | C16:1 (mono UFA) | YES |
| Oleic | C18:1 cis-9 (mono UFA) | YES |
| cis-Vaccenic | C18:1 cis-11 (mono UFA) | YES |
| Elaidic | C18:1 trans-9 (mono UFA) | YES |
| Linoleic | C18:2 (poly UFA) | YES |
| dihydrosterculic | cyc-C19:0, 9-(CFA of oleic) | YES |
| cis 11-eicosenoic | C20:1 cis-11 (mono UFA) | YES |
| cis 13 eicosenoic | C20:1 cis-13 (mono UFA) | no |
| cis-11,14- | C20:2 (poly UFA) | Partial growth |
| Eicosadienoic | | Very slight growth |
| Erucic | C22:1 cis-13 (mono UFA) | |

None of the saturated fatty acids tested supported growth of BP63, while several unsaturated fatty acids in addition to oleic acid allowed growth of the BP63, as expected for an unsaturated fatty acid auxotroph.

Growth of the BP63 in the Presence of Isobutanol

The purpose of these experiments was to see if the requirement for oleic acid changed in the presence of isobutanol. Semi-synthetic LAB medium, pH6, supplemented with 75 μg/mL uracil, 2.5 μg/mL hematin was used along with a series of varying concentrations of isobutanol and oleic acid. Oleic was added to the final concentrations of 0, 10, 20, 30, 40, and 50 mg/L. Isobutanol was added to the final concentrations of 0, 1.0, 1.5, 2.0, 2.5, and 3% (w/v). 2.5 mL of media was inoculated with 124 of the BP63 working stock. The cultures were incubated at 30° C. without shaking for 18 hours. At 18 hours the OD₆₀₀ was measured. The results for the fabZ1 mutant strain, BP63, are shown in Table 10.

TABLE 10
Growth of the BP63 (ΔfabZ1) in the presence of isobutanol and oleic acid.

| [oleic acid] | Growth (OD₆₀₀) of BP63 (ΔfabZ1) in iso-butanol | | | | | |
|---|---|---|---|---|---|---|
| mg/liter | 0% | 1% | 1.5% | 2% | 2.5% | 3% |
| 0 | 0.0607 | 0.0383 | 0.0385 | 0.0402 | 0.038 | 0.0351 |
| 10 | 0.0835 | 0.0829 | 0.0601 | 0.0701 | 0.0684 | 0.0397 |
| 20 | 0.1046 | 0.2291 | 0.2375 | 0.068 | 0.0594 | 0.0399 |
| 30 | 0.1526 | 0.7686 | 0.7137 | 0.402 | 0.1606 | 0.0995 |
| 40 | 0.2976 | 1.2315 | 0.8852 | 0.8012 | 0.1793 | 0.1358 |
| 50 | 1.181 | 1.2567 | 1.257 | 0.5464 | 0.1654 | 0.1185 |

It is clear that when oleic acid was supplied at sub-optimal levels, the presence of isobutanol enhanced the growth of the fabZ1 mutant. For example, 30 mg/liter oleic acid in the absence of isobutanol allowed growth to an OD₆₀₀ of only 0.153. While addition of 1% or 1.5% isobutanol, allowed growth to OD₆₀₀ of 0.769 and 0.714, respectively.

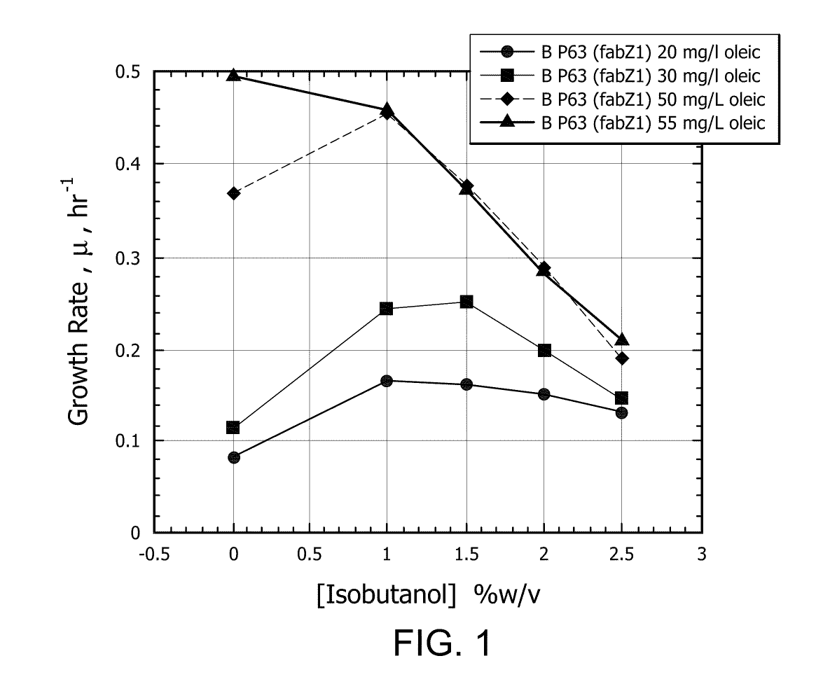

To follow up observation of isobutanol stimulated growth of the fabZ1 mutant, shake flask experiments were done in semi-synthetic LAB medium, pH6, with added uracil, hematin as above and using and an initial OD₆₀₀ of 0.1. Four sets of conditions were prepared. For the first set, 20 mg/l oleic acid was added and isobutanol was added to 0, 1, 1.5, 2 and 2.5% final concentration. In the second set of flasks, 30 mg/L of oleic acid was added to the medium and the following final isobutanol concentrations were used: 0, 1, 1.5, 2, 2.5, and 3% w/v. The third and fourth set of the shake flask cultures were done at oleic acid concentrations of 50 and 55 mg/L. These flasks were placed in a shaking water bath at 30° C. at 80 RPM. Samples were taken at 2, 3, 4, and 5 hrs and the OD₆₀₀ was measured. Growth rates for the fabZ1 mutant BP63, calculated from plots of the natural log of the OD₆₀₀ vs. time are shown in the FIG. 1.

Thus, the isobutanol stimulated growth of the fabZ1 mutant strain BP63 at suboptimal concentrations of oleic acid was confirmed. The growth rate of BP63 at 55 mg/liter oleic acid was essentially identical to that of the parental strain, BP15, at all concentrations of isobutanol (data for BP15 not shown).

### Example 6

### Expression of fabZ1 Gene Under the Control of clpL Promoter

The purpose of this example is to describe plasmid-borne expression of fabZ1 from a weak promoter.

The expression vector pFP996 PclpL (SEQ ID NO: 72) was used to express the fabZ1 gene. As described earlier the plasmid pFP996 is a shuttle vector that can replicate in both E. coli and L. plantarum. Vector pFP996 PclpL contains the PclpL promoter from L. plantarum for gene expression (nt 5350 to 5682). The fabZ1 gene from L. plantarum strain PNO512 was amplified with primer set fabZ/(S.D.)-F(SpeI) and fabZ1-R(BgIII/XmaI) (SEQ ID NO: 69 and SEQ ID NO: 70) using genomic DNA as the template. The PCR product was digested with restriction enzyme SpeI and Xmal and fragment obtained was ligated to the corresponding sites in pFP996 PclpL. The ligation mixture was transformed into E. coli TOP10 cells and cells were plated on LB plates supplemented with ampicillin (100 μg/ml). The positive clones were screened using primer set ClpL-F (SEQ ID NO: 71) and fabZ1-R(BgIII/XmaI) (SEQ ID NO: 70). Two positive clones identified were confirmed by sequencing and they were designated as pFP996 PclpL-fabZ1#1 and pFP996 PclpL-fabZ1#2 represented by SEQ ID NO: 73. The latter plasmid was transformed into strain BP15 (ΔpyrF fabZ1⁺) and BP63 (ΔpyrFΔfabZ1). The resultant strains were named as follows:

PN2043 and PN2044 represent BP15 (pFP996 PclpL-fabZ1#2); PN2048, PN2049, PN2050, and PN2051 represent BP63(pFP996 PclpL-fabZ1#2)

### Example 7

### Increased Membrane Saturated Fatty Acid Content of L. plantarum ΔfabZ1 Carrying Plasmid Borne fabZ1 Driven by the Promoter PclpL

The purpose of this example is to demonstrate genetic modification of L. plantarum that results in increased saturated fatty acids in membrane lipids without feeding exogenous free fatty acids.

Strains PN2043. PN2044 (BP15: pFP996 PclpL-fabZ1#2) and strains PN2048, PN2049, PN2050, and PN2051 (BP63: pFP996 PclpL-fabZ1#2) described in Example 6 were grown in semi-synthetic LAB media, pH6, with 75 μg/mluracil but lacking exogenous free fatty acids and the BSA carrier. Samples for inoculation were prepared by taking a single colony from a plate and resuspended in LAB media. The OD₆₀₀ of this was read and they were then diluted into 40 ml LAB medium to a starting OD₆₀₀ of 0.1. The samples were grown at 37° C. until they reached an OD₆₀₀ of approximately 0.6 (24 hours for PN2048, PN2049, PN2050, and PN2051). The cultures were harvested after reaching the desired OD₆₀₀ by centrifugation and the supernatant was removed. The pellets were washed in PBS four times to remove any residual medium. Membrane composition was analyzed as described in General Methods. The results of FAME analyses shown in Table 11.

TABLE 11
Weight % membrane fatty acids in strains with low level
expresssion of fabZ1 from plasmid and control strains.

| | | Strain | | | | | |
|---|---|---|---|---|---|---|---|
| | | PN2043 | PN2044 | PN2048 | PN2049 | PN2050 | PN2051 |
| | | Genotype | | | | | |
| | | fabZ1⁺/ | fabZ1⁺/ | ΔfabZ1/ | ΔfabZ1/ | ΔfabZ1/ | ΔfabZ1/ |
| | | pFabZ1 | pFabZ1 | pFabZ1 | pFabZ1 | pFabZ1 | pFabZ1 |
| Fatty | C14:0 | 0.4 | 0.4 | 1.3 | 1.8 | 1.3 | 1.3 |
| Acid | C16:0 | 29.9 | 27.6 | 33.1 | 32.7 | 31.1 | 32.0 |
| | C16:1 | 4.8 | 8.2 | 4.6 | 3.9 | 2.8 | 2.9 |
| | C18:0 | 8.7 | 8.2 | 12.3 | 17.5 | 13.2 | 13.4 |
| | C18:1 | 39.8 | 35.9 | 27.9 | 16.0 | 16.8 | 16.2 |
| | cyc- | 8.9 | 12.3 | 13.4 | 15.5 | 17.7 | 21.0 |
| | C19:0 | | | | | | |
| | Total | 39.0 | 36.2 | 46.7 | 52.0 | 45.6 | 46.7 |
| | satu- | | | | | | |
| | rated | | | | | | |

The total saturated fatty acid in the membranes of PN2048, PN2049, PN2050 and PN2051 was increased as compared with that in strains PC2043 and PN2044. Thus, expression of fabZ1 from the promoter PclpL in a host with a deleted fabZ1 gene was an effective genetic modification to increase saturated fatty acids L. plantarum membranes.

### Example 8

### Promoter Replacement in L. plantarum PN0512ΔpyrF to Weaken Expression of fabZ1 (Prophetic)

The purpose of this prophetic example is to describe how chromosomal modifications of L. plantarum can be constructed leading to increased saturated fatty acids in membrane lipids without feeding exogenous free fatty acids.

The chromosomal fabZ1 promoter region of L. plantarum, PfabZ1, is replaced with a weaker promoter region, PclpL, in order to decrease, but not eliminate expression of fabZ1 from the chromosome. The fabZ1 promoter replacement is constructed using the two-step homologous recombination procedure described in Example 4. The fabZ1 promoter region, from 270 bp upstream of the fabZ1 start codon through 21 bp upstream of the fabZ1 start codon (leaving the ribosome binding site), is deleted and replaced with the clpL promoter region, including 265 bp upstream of the clpL start codon through 16 bp upstream of the clpL start codon (not including the ribosome binding site).

The homologous arms and PclpL are amplified from L. plantarum strain PN0512 genomic DNA. The PfabZ1 left homologous arm is amplified using oligonucleotides left-arm-up (SEQ ID NO: 74) containing a BgIII restriction site and left-arm-down (SEQ ID NO: 75) containing an XhoI restriction site. The PfabZ1 right homologous arm is amplified using oligonucleotides right-arm-up (SEQ ID NO: 76) containing an XmaI restriction site and right-arm-down (SEQ ID NO: 77) containing a BsrGI restriction site. The PfabZ1 left homologous arm is digested with BgIII and XhoI and the PfabZ1 right homologous arm is digested with XmaI and BsrGI. The two homologous arms are ligated with T4 DNA Ligase into the corresponding restriction sites of pFP996pyrFΔerm after digestion with the appropriate restriction enzymes to create vector pFP996pyrFΔerm-PfabZ1 arms. PclpL is amplified using oligonucleotides PclpL-up (SEQ ID NO: 78) containing an XhoI restriction site and PclpL-down (SEQ ID NO: 79) containing an XmaI restriction site. PclpL is digested with XhoI and Xmal. PclpL is ligated with T4 DNA Ligase into the corresponding restriction sites of pFP996pyrFΔerm-PfabZ1 arms after digestion with the appropriate restriction enzymes to create vector pFP996pyrFΔerm-PclpL-PfabZ1 arms. BP15 (described in Example 4) is transformed with the pFP996pyrFΔerm-PclpL-PfabZ1 arms construct by electroporation. Transformants are grown at 30° C. in minimal medium without uracil for approximately 10 generations in an anaerobic box containing a Pack-Anaero sachet, followed by growth at 42° C. for approximately 20 generations by serial inoculations in minimal medium without uracil in an anaerobic box containing a Pack-Anaero sachet. Cultures are plated on minimal medium without uracil and isolates are screened by colony PCR for a single cross-over with chromosomal specific oligonucleotide PfabZ1 chromosome-up (SEQ ID NO: 80) and plasmid specific oligonucleotide oBP42 (SEQ ID NO: 66). Single cross-over integrants are grown at 37° C. for approximately 40 generations by serial inoculations under non-selective conditions in Lactobacilli MRS medium. Cultures are plated on MRS medium and isolates are patched to MRS plates, grown at 37° C., and then patched onto minimal medium plates without uracil. Uracil auxotroph (double cross-over) isolates are screened by colony PCR for the presence of PclpL in the chromosome using oligonucleotides PfabZ1 chromosome-up (SEQ ID NO:80) and PclpL-down (SEQ ID NO: 79). A PCR product of 1555 bp indicates that the PfabZ1 promoter has been replaced with the PclpL promoter. The promoter replacement is confirmed by sequencing the region after PCR amplification using chromosomal specific oligonucleotides PfabZ1 chromosome-up (SEQ ID NO: 80) and PfabZ1 chromosome-down (SEQ ID NO: 81). The resulting strain is named PN0512ΔpyrF_PclpL-fabZ.

Strains PN0512ΔpyrF_PclpL-fabZ and its parental strain, BP15, are grown in semi-synthetic LAB media, pH6, with 75 μg/mluracil but lacking exogenous free fatty acids and the BSA carrier. Samples for inoculation are prepared by taking a single colony from a plate and resuspending in LAB media. The OD₆₀₀ of this is read and they are diluted into 40 ml of LAB media to a starting OD of 0.1. The samples are grown at 37° C. until they reached an OD₆₀₀ of approximately 0.6. Once they reached the desired OD₆₀₀, they are harvested, spun down and pellets are washed in PBS 4 times to remove any residual media. Membrane composition is analyzed as described in General Methods. The results of FAME analyses show that strains PN0512ΔpyrF_PclpL-fabZ has more saturated fatty acids in the membrane than does strain BP15.

### Example 9

### Optimization of fabZ1 Expression

The slow growth of strains PN2048, PN2049, PN2050, and PN2051 (PN0512 ΔpyrF ΔfabZ1 or carrying plasmid pFP996 PclpL-fabZ1#2) suggested that clpL promoter led to a low level of expression of fabZ1gene as compared to the wild type. In order to achieve a medium level of expression of fabZ1 for increased growth rate but still resulting in increased saturated fatty acids in membrane lipids, stronger promoters are necessary. For example, promoters for cydA, agrB and atpB from L. plantarum may be used. Specifically, the clpL promoter region in vector pFP996 PclpL-fabZ1 is replaced by these three alternative promoters. The clpL promoter region is flanked by two unique restriction sites EcoRI and SpeI.

### Expression of Plasmid-Borne fabZ1 Gene Under the Control of Stronger Promoters (Prophetic)

The purpose of this prophetic example is to describe how to use alternative promoters for plasmid-borne expression of fabZ1.

Primers with restriction sites EcoR1 and SpeI are designed and used to amplify the cydA promoter region (SEQ ID NO: 82). After digestion, the PCR product is ligated to the corresponding sites in vector pFP996 PclpL-fabZ1. The resulting clones are then transferred into L. plantarum strain BP63 (ΔfabZ1). Similar strategies are used to expression fabZ1 gene under the control of agrB (SEQ ID NO: 84) and atpB (SEQ ID NO:83) promoters respectively. Strains with plasmid-borne expression of fabZ1 from the promoters for cydA, agrB and atpB and a control strain, BP15 (fabZ1⁺) are grown in semi-synthetic LAB media, pH6, with 75 μg/ml uracil, but lacking exogenous free fatty acids and the BSA carrier. Samples for inoculation are prepared by taking a single colony from a plate and resuspending in LAB media. The OD₆₀₀ is read and they are diluted into 40 ml of LAB media to a starting OD₆₀₀ of 0.1. The samples are grown at 37° C. until they reached an OD₆₀₀ of approximately 0.6. Upon reaching the desired OD₆₀₀, the cultures are harvested by centrifugation and pellets are washed in PBS four times to remove any residual medium. Membrane composition is analyzed as described in General Methods. The results of FAME analyses show that strains with plasmid-borne expression of fabZ1 in a fabZ1 deletion host have more saturated fatty acids in the membrane than does the control strain. The growth rate of these strains and strains PN2048, PN2049, PN2050 and PN2051 (described in example 6) are analyzed and a strain with the optimum balance of elevated membrane saturated fatty acids and a reasonable growth rate is selected and named BP63 (pfabZ1opt).

### Example 10

### Expression of an Isobutanol Biosynthetic Pathway in Lactobacillus plantarum with Increased Membrane Saturated Fatty Acids Due to Decreased Chromosomal Expression of fabZ1 (Prophetic)

The purpose of this prophetic Example is to describe how to express an isobutanol biosynthetic pathway in a Lactobacillus plantarum strain that has higher levels of saturated fatty acids in the membrane lipids, such as PN0512ΔpyrF_PclpL-fabZ1 (described in Example 8). The five genes of the isobutanol pathway, encoding five enzyme activities, are divided into two operons for expression. The budB, ilvD and kivD genes, encoding the enzymes acetolactate synthase, acetohydroxy acid dehydratase, and branched-chain α-keto acid decarboxylase, respectively, are integrated into the chromosome of Lactobacillus plantarum by homologous recombination using the method described by Hols et al. (Appl. Environ. Microbiol. 60:1401-1413 (1994)). The remaining two genes of the isobutanol biosynthetic pathway (ilvC and bdhB, encoding the enzymes acetohydroxy acid reductoisomerase and butanol dehydrogenase, respectively) are cloned into an expression plasmid and transformed into the Lactobacillus strain carrying the integrated isobutanol genes. Lactobacillus plantarum is grown in MRS medium (Difco Laboratories, Detroit, Mich.) at 37° C., and chromosomal DNA is isolated as described by Moreira et al. (BMC Microbiol. 5:15 (2005)).

### Integration

The budB-ilvD-kivD cassette under the control of the synthetic P11 promoter (Rud et al., Microbiology 152:1011-1019 (2006)) is integrated into the chromosome of Lactobacillus plantarum PN0512ΔpyrF_PclpL-fabZ1 at the IdhL1 locus by homologous recombination. To build the IdhL integration targeting vector, a DNA fragment from Lactobacillus plantarum (Genbank NC_{—}004567) with homology to IdhL is PCR amplified with primers LDH EcoRV F (SEQ ID NO:85) and LDH AatIIR (SEQ ID NO:86). The 1986 bp PCR fragment is cloned into pCR4Blunt-TOPO and sequenced. The pCR4Blunt-TOPO-IdhL1 clone is digested with EcoRV and AatII releasing a 1982 bp IdhL1 fragment that is gel-purified. The integration vector pFP988 is a Bacillus integration vector provided as SEQ ID NO: 87. pFP988 contains an E. coli replicon from pBR322, an ampicillin antibiotic marker for selection in E. coli and two sections of homology to the sacB gene in the Bacillus chromosome that directs integration of the vector and intervening sequence by homologous recombination. pFP988 is digested with HindIII and treated with Klenow DNA polymerase to blunt the ends. The linearized plasmid is then digested with AatII and the 2931 bp vector fragment is gel purified. The EcoRV/AatII IdhL1 fragment is ligated with the pFP988 vector fragment and transformed into E. coli Top10 cells. Transformants are selected on LB agar plates containing ampicillin (100 μg/mL) and are screened by colony PCR to confirm construction of pFP988-IdhL.

To add a selectable marker to the integrating DNA, the Cm resistance gene with its promoter is PCR amplified from pC194 (GenBank NC_{—}002013) with primers Cm F (SEQ ID NO:88) and Cm R (SEQ ID NO: 89), amplifying a 836 bp PCR product. This PCR product is cloned into pCR4Blunt-TOPO and transformed into E. coli Top10 cells, creating pCR4Blunt-TOPO-Cm. After sequencing to confirm that no errors are introduced by PCR, the Cm cassette is digested from pCR4Blunt-TOPO-Cm as an 828 bp MluI/SwaI fragment and is gel purified. The IdhL-homology containing integration vector pFP988-IdhL is digested with MluI and Swal and the 4740 bp vector fragment is gel purified. The Cm cassette fragment is ligated with the pFP988-IdhL vector creating pFP988-DldhL::Cm.

The budB-ilvD-kivD cassette, described in US 2007-0092957 A1, includes the Klebsiella pneumoniae budB coding region, the E. coli ilvD coding region, and the codon optimized Lactococcus lactis kivD coding region from pFP988DssPspac-budB-ilvD-kivD. The budB-ilvD-kivD cassette is modified to replace the amylase promoter with the synthetic P11 promoter. Then, the whole operon is moved into pFP988-DldhL::Cm. The P11 promoter is constructed by oligonucleotide annealing with primers P11 F-Stul (SEQ ID NO:90) and P11 R-SpeI (SEQ ID NO: 91). The annealed oligonucleotide is gel-purified on a 6% Ultra PAGE gel (Embi Tec, San Diego, Calif.). The plasmid pFP988DssPspac-budB-ilvD-kivD, containing the amylase promoter, is digested with StuI and SpeI and the resulting 10.9 kbp vector fragment is gel-purified. The isolated P11 fragment is ligated with the digested pFP988DssPspac-budB-ilvD-kivD to create pFP988-P11-budB-ilvD-kivD. Plasmid pFP988-P11-budB-ilvD-kivD is then digested with StuI and BamHI and the resulting 5.4 kbp P11-budB-ilvD-kivD fragment is gel-purified. pFP988-DldhL::Cm is digested with HpaI and BamHI and the 5.5 kbp vector fragment isolated. The budB-ilvD-kivD operon is ligated with the integration vector pFP988-DldhL::Cm to create pFP988-DldhL-P11-budB-ilvD-kivD::Cm.

Integration of pFP988-DldhL-P11-budB-ilvD-kivD::Cm into L. plantarum PN0512ΔpyrF_PclpL-fabZ1 to form L. plantarum PN0512ΔpyrF_PclpL-fabZ1 ΔldhL1::budB-ilvD-kivD::Cm comprising exogenous budB, ilvD, and kivD genes.

Electrocompetent cells of L. plantarum are prepared as described by Aukrust, T. W., et al. (In: Electroporation Protocols for Microorganisms; Nickoloff, J. A., Ed.; Methods in Molecular Biology, Vol. 47; Humana Press, Inc., Totowa, N.J., 1995, pp 201-208). After electroporation, cells are outgrown in MRSSM medium (MRS medium supplemented with 0.5 M sucrose and 0.1 M MgCl₂) as described by Aukrust et al. supra for 2 h at 37° C. without shaking. Electroporated cells are plated for selection on MRS plates containing chloramphenicol (10 μg/mL) and incubated at 37° C. Transformants are initially screened by colony PCR amplification to confirm integration, and initial positive clones are then more rigorously screened by PCR amplification with a battery of primers.

Plasmid Expression of ilvC and bdhB Genes.

The remaining two isobutanol genes under the control of the L. plantarum IdhL promoter (Ferain et al., J. Bacteriol. 176:596-601 (1994)) are expressed from plasmid pTRKH3 (O'Sullivan D J and Klaenhammer TR, Gene 137:227-231 (1993)). The IdhL promoter is PCR amplified from the genome of L. plantarum ATCC BAA-793 using primers PldhL F-HindIII (SEQ ID NO: 92) and PldhL R-BamHI (SEQ ID NO: 93). The 411 bp PCR product is cloned into pCR4Blunt-TOPO and sequenced. The resulting plasmid, pCR4Blunt-TOPO-PldhL is digested with HindIII and BamHI releasing the PldhL fragment

Plasmid pTRKH3 is digested with SphI and partially digested with HindIII. The gel-purified approximately 7 Kb vector fragment is ligated with the PldhL fragment and the gel-purified 2.4 kbp BamHI/SphI fragment containing ilvC(B.s.)-bdhB, which includes the Bacillus subtilis ilvC coding region and the Clostridium acetobutylicum bdhB coding region from a Bacillus expression plasmid pBDPgroE-ilvC(B.s.)-bdhB (described in US 2007-0092957 A1) in a three-way ligation. The ligation mixture is transformed into E. coli Top 10 cells and transformants are grown on Brain Heart Infusion (BHI, Difco Laboratories, Detroit, Mich.) plates containing erythromycin (150 μg/L). Transformants are screened by PCR to confirm construction. The resulting plasmid is pTRKH3-ilvC(B.s.)-bdhB. This plasmid is transformed into L. plantarum PN0512ΔpyrF_PclpL-fabZ1 ΔldhL1::budB-ilvD-kivD::Cm by electroporation, as described above.

L. plantarum PN0512ΔpyrF_PclpL-fabZ1 ΔldhL1::budB-ilvD-kivD::Cm containing pTRKH3-ilvC(B.s.)-bdhB is inoculated into a 250 mL shake flask containing 50 mL of MRS medium plus erythromycin (10 μg/mL) and grown at 37° C. for 18 to 24 h without shaking, after which isobutanol is detected by HPLC or GC analysis. Higher titers of isobutanol are obtained from a control strain similarly constructed but with wildtype expression of fabZ1.

### Example 11

### Expression of an Isobutanol Biosynthetic Pathway in Lactobacillus plantarum with Plasmid-Borne Expression of fabZ1 for Increased Membrane Saturated Fatty Acids (Prophetic)

The purpose of this prophetic example is to describe how to express an isobutanol biosynthetic pathway in a Lactobacillus plantarum strain that has higher levels of saturated fatty acids in the membrane lipids due to plasmid-borne expression of fabZ1 in a fabZ1 deletion host, such as BP63: pfabZ1opt (described in Example 9).

The five genes of the isobutanol pathway, encoding five enzyme activities, are divided into two operons for expression. The budB, ilvD and kivD genes, encoding the enzymes acetolactate synthase, acetohydroxy acid dehydratase, and branched-chain α-keto acid decarboxylase, respectively, are integrated into the chromosome of Lactobacillus plantarum by homologous recombination using the method described by Hols et al. (Appl. Environ. Microbiol. 60:1401-1413 (1994)). The remaining two genes of the isobutanol biosynthetic pathway (ilvC and bdhB, encoding the enzymes acetohydroxy acid reductoisomerase and butanol dehydrogenase, respectively) are cloned into an expression plasmid and transformed into the Lactobacillus strain carrying the integrated isobutanol genes. Lactobacillus plantarum is grown in MRS medium (Difco Laboratories, Detroit, Mich.) at 37° C., and chromosomal DNA is isolated as described by Moreira et al. (BMC Microbiol. 5:15 (2005)).

### Integration

The budB-ilvD-kivD cassette under the control of the synthetic P11 promoter (Rud et al., Microbiology 152:1011-1019 (2006)) is integrated into the chromosome of Lactobacillus plantarum ATCC BAA-793 (NCIMB 8826) at the IdhL1 locus by homologous recombination. To build the IdhL integration targeting vector, a DNA fragment from Lactobacillus plantarum (Genbank NC_{—}004567) with homology to IdhL is PCR amplified with primers LDH EcoRV F (SEQ ID NO:85) and LDH AatIIR (SEQ ID NO:86). The 1986 bp PCR fragment is cloned into pCR4Blunt-TOPO and sequenced. The pCR4Blunt-TOPO-IdhL1 clone is digested with EcoRV and AatII releasing a 1982 bp IdhL1 fragment that is gel-purified. The integration vector pFP988, pFP988-IdhL and pFP988-DldhL::Cm and pFP988-DldhL-P11-budB-ilvD-kivD::Cm are described in Example 10.

Integration of pFP988-DldhL-P11-budB-ilvD-kivD::Cm into L. plantarum PN0512ΔpvrFΔfabZ1 to Form L. plantarum PN0512ΔpvrFΔfabZ1 IdhL1::budB-ilvD-kivD::Cm Comprising Exogenous budB, ilvD, and kivD Genes.

Electrocompetent cells of L. plantarum are prepared as described by Aukrust, T. W., et al. (In: Electroporation Protocols for Microorganisms; Nickoloff, J. A., Ed.; Methods in Molecular Biology, Vol. 47; Humana Press, Inc., Totowa, N.J., 1995, pp 201-208). After electroporation, cells are outgrown in MRSSM medium (MRS medium supplemented with 0.5 M sucrose and 0.1 M MgCl₂) as described by Aukrust et al. supra for 2 h at 37° C. without shaking. Electroporated cells are plated for selection on MRS plates containing chloramphenicol (10 μg/mL) and incubated at 37° C. Transformants are initially screened by colony PCR amplification to confirm integration, and initial positive clones are then more rigorously screened by PCR amplification with a battery of primers.

Plasmid Expression of ilvC, bdhB and cti Genes.

The remaining two isobutanol genes under the control of the L. plantarum IdhL promoter (Ferain et al., J. Bacteriol. 176:596-601 (1994)) and fabZ1 under the control of the optimal promoter as described in Example 9 are expressed from plasmid pTRKH3 (O'Sullivan D J and Klaenhammer T R, Gene 137:227-231 (1993)). The IdhL promoter is PCR amplified from the genome of L. plantarum ATCC BAA-793 using primers PldhL F-HindIII (SEQ ID NO: 92) and PldhL R-BamHI (SEQ ID NO: 93). The 411 bp PCR product is cloned into pCR4Blunt-TOPO and sequenced. The resulting plasmid, pCR4Blunt-TOPO-PldhL is digested with HindIII and BamHI releasing the PldhL fragment

The plasmid pTRKH3-ilvC(B.s.)-bdhB described in Example 10, is digested with SphI and treated with calf intestinal alkaline phosphatase. A PCR product containing the optimal promoter driving fabZ1 is amplified from pfabZ1opt (Example 9) with primers carrying SphI restriction sites and digested with SphI. This fragment is ligated to the SphI-digested pTRKH3-ilvC(B.s.)-bdhB. The ligation mixture is transformed into E. coli Top 10 cells and transformants are grown on Brain Heart Infusion (BHI, Difco Laboratories, Detroit, Mich.) plates containing erythromycin (150 μg/L). The transformants are screened by PCR and one with the fabZ1 gene in the same orientation as i/vC and bdhB is retained and named pTRKH3-ilvC(B.s.)-bdhB-fabZ1. This plasmid is transformed into L. plantarum PN0512ΔpyrFΔfabZ1 IdhL1::budB-ilvD-kivD::Cm by electroporation, as described above.

L. plantarum PN0512ΔpyrFΔfabZ1 IdhL1::budB-ilvD-kivD::Cm containing pTRKH3-ilvC(B.s.)-bdhB-fabZ1 is inoculated into a 250 mL shake flask containing 50 mL of MRS medium plus erythromycin (10 μg/mL) and grown at 37° C. for 18 to 24 h without shaking, after which isobutanol is detected by HPLC or GC analysis. Higher titers of isobutanol are obtained from this strain than from a similarly constructed control strain but with wild type expression of fabZ1.

### Example 12 (Prophetic)

### Methods for Determining Isobutanol Concentration in Culture Media

The concentration of isobutanol in the culture media can be determined by a number of methods known in the art. For example, a specific high performance liquid chromatography (HPLC) method utilized a Shodex SH-1011 column with a Shodex SH-G guard column, both purchased from Waters Corporation (Milford, Mass.), with refractive index (R1) detection. Chromatographic separation was achieved using 0.01 M H₂SO₄ as the mobile phase with a flow rate of 0.5 ml/min and a column temperature of 50° C. Isobutanol had a retention time of 46.6 min under the conditions used. Alternatively, gas chromatography (GC) methods are available. For example, a specific GC method utilized an HP-INNOWax column (30 m×0.53 mm id, 1 μm film thickness, Agilent Technologies, Wilmington, Del.), with a flame ionization detector (FID). The carrier gas was helium at a flow rate of 4.5 mL/min, measured at 150° C. with constant head pressure; injector split was 1:25 at 200° C.; oven temperature was 45° C. for 1 min, 45 to 220° C. at 10° C./min, and 220° C. for 5 min; and FID detection was employed at 240° C. with 26 mL/min helium makeup gas. The retention time of isobutanol was 4.5 min.

What is claimed is:

1. A recombinant bacterial cell for the production of butanol comprising:
i) a butanol biosynthetic pathway, and
ii) a cell membrane having at least about a 10% increase in total cell membrane saturated fatty acid content as compared with a parent bacterial cell;
wherein the butanol biosynthetic pathway comprises at least one gene that is heterologous to the bacterial cell.

2. The bacterial cell of claim 1 further comprising a genetic modification in a gene of an unsaturated fatty acid biosynthetic pathway wherein said genetic modification increases the total cell membrane saturated fatty acid content.

3. The bacterial cell of claim 1 wherein the bacterial cell is member of a genus selected from the group consisting of Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Lactococcus, Pediococcus, and Leuconostoc.

4. The bacterial cell of claim 1 wherein the butanol biosynthetic pathway is an isobutanol biosynthetic pathway.

5. A recombinant lactobacillus cell comprising a genetic modification in at least one of fabA, fabM, fabN, fabZ or fabZ1 and having at least about a 10% increase in total cell membrane saturated fatty acids as compared with a wild-type lactobacillus cell.

6. The lactobacillus cell of claim 5 having increased tolerance to butanol as compared with the parent lactobacillus cell.

7. The lactobacillus cell of claim 5 further comprising a butanol biosynthetic pathway.

8. The lactobacillus cell of claim 6 wherein at least one substrate to product conversion of the butanol biosynthetic pathway is catalyzed by a protein encoded by a heterologous polynucleotide.

9. A recombinant lactobacillus cell comprising:
(i) decreased activity for isomerization of trans-2-decenoyl-Acyl Carrier Protein to cis-3-decenoyl-Acyl Carrier Protein; and
(ii) at least 10% increase in total cell membrane saturated fatty acids as compared with a wild-type lactobacillus cell.

10. A method for increasing the tolerance of a bacterial cell to butanol comprising increasing the concentration of saturated fatty acids in the membrane of the bacterial cell whereby the tolerance of the bacterial cell to butanol is increased as compared with a bacterial cell where the concentration of saturated fatty acids in the membrane has not been increased.

11. The method of claim 10 wherein increasing the concentration of saturated fatty acids in the membrane of the bacterial cell comprises growing the bacterial cell in media containing at least one saturated fatty acid.

12. The method of claim 10 wherein increasing the concentration of saturated fatty acids in the membrane of the bacterial cell comprises introduction of a genetic modification in a gene of an unsaturated fatty acid biosynthetic pathway.

13. The method of claim 10 wherein the bacterial cell is member of a genus selected from the group consisting of Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Lactococcus, Pediococcus, and Leuconostoc.

14. The method of claim 11 wherein the at least one saturated fatty acid is C14:0, C15:0; C16:0, C17:0, C18:0, C19:0 or C20:0.

15. The method of claim 12 wherein the gene of an unsaturated fatty acid biosynthetic pathway is fabA, fabM, fabN, fabZ, or fabZ1.

16. The method of claim 12 wherein the gene of an unsaturated fatty acid biosynthetic pathway encodes a protein that catalyzes isomerization of trans-2-decenoyl-Acyl Carrier Protein to cis-3-decenoyl-Acyl Carrier Protein.

17. The method of claim 12 wherein the genetic modification in a gene of an unsaturated fatty acid biosynthetic pathway results in reduced or eliminated expression of the protein encoded by the fabZ1 gene.

18. The method of claim 12 wherein the genetic modification comprises a deletion.

19. The method of claim 12 wherein the genetic modification comprises expressing a gene of an unsaturated fatty acid biosynthetic pathway under the control of a non-native promoter.

20. The method of claim 19 wherein the gene of an unsaturated fatty acid biosynthetic pathway is fabZ1.

21. The method of claim 16 wherein the product of the gene of unsaturated fatty acid biosynthetic pathway additionally catalyzes β-hydroxyacyl-ACP dehydratase activity.

22. The method of claim 12 wherein the genetic modification comprises a deletion of the native fabZ1 gene and further comprises expression a fabZ1 gene under a weak promoter.


SEQUENCE LISTING (129 sequences)

SEQ ID NO: 1 - DNA, 1179 bp, Clostridium acetobutylicum
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 2 - protein, 392 aa, Clostridium acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 3 - DNA, 1179 bp, Clostridium acetobutylicum
atgagagatgtagtaatagtaagtgctgtaagaactgcaataggagcatatggaaaaacattaaaggatgtacctgcaacagagttaggagctatagtaataaaggaagctgtaagaagagctaatataaatccaaatgagattaatgaagttatttttggaaatgtacttcaagctggattaggccaaaacccagcaagacaagcagcagtaaaagcaggattacctttagaaacacctgcgtttacaatcaataaggtttgtggttcaggtttaagatctataagtttagcagctcaaattataaaagctggagatgctgataccattgtagtaggtggtatggaaaatatgtctagatcaccatatttgattaacaatcagagatggggtcaaagaatgggagatagtgaattagttgatgaaatgataaaggatggtttgtgggatgcatttaatggatatcatatgggagtaactgcagaaaatattgcagaacaatggaatataacaagagaagagcaagatgaattttcacttatgtcacaacaaaaagctgaaaaagccattaaaaatggagaatttaaggatgaaatagttcctgtattaataaagactaaaaaaggtgaaatagtctttgatcaagatgaatttcctagattcggaaacactattgaagcattaagaaaacttaaacctattttcaaggaaaatggtactgttacagcaggtaatgcatccggattaaatgatggagctgcagcactagtaataatgagcgctgataaagctaacgctctcggaataaaaccacttgctaagattacttcttacggatcatatggggtagatccatcaataatgggatatggagctttttatgcaactaaagctgccttagataaaattaatttaaaacctgaagacttagatttaattgaagctaacgaggcatatgcttctcaaagtatagcagtaactagagatttaaatttagatatgagtaaagttaatgttaatggtggagctatagcacttggacatccaataggtgcatctggtgcacgtattttagtaacattactatacgctatgcaaaaaagagattcaaaaaaaggtcttgctactctatgtattggtggaggtcagggaacagctctcgtagttgaaagagactaa

SEQ ID NO: 4 - protein, 392 aa, Clostridium acetobutylicum
MRDVVIVSAVRTAIGAYGKTLKDVPATELGAIVIKEAVRRANINPNEINEVIFGNVLQAGLGQNPARQAAVKAGLPLETPAFTINKVCGSGLRSISLAAQIIKAGDADTIVVGGMENMSRSPYLINNQRWGQRMGDSELVDEMIKDGLWDAFNGYHMGVTAENIAEQWNITREEQDEFSLMSQQKAEKAIKNGEFKDEIVPVLIKTKKGEIVFDQDEFPRFGNTIEALRKLKPIFKENGTVTAGNASGLNDGAAALVIMSADKANALGIKPLAKITSYGSYGVDPSIMGYGAFYATKAALDKINLKPEDLDLIEANEAYASQSIAVTRDLNLDMSKVNVNGGAIALGHPIGASGARILVTLLYAMQKRDSKKGLATLCIGGGQGTALVVERD

SEQ ID NO: 5 - DNA, 849 bp, Clostridium acetobutylicum
atgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataa

SEQ ID NO: 6 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 7 - DNA, 786 bp, Clostridium acetobutylicum
atggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagatag

SEQ ID NO: 8 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 9 - DNA, 1197 bp, Clostridium acetobutylicum
atgatagtaaaagcaaagtttgtaaaaggatttatcagagatgtacatccttatggttgcagaagggaagtactaaatcaaatagattattgtaagaaggctattgggtttaggggaccaaagaaggttttaattgttggagcctcatctgggtttggtcttgctactagaatttcagttgcatttggaggtccagaagctcacacaattggagtatcctatgaaacaggagctacagatagaagaataggaacagcgggatggtataataacatattttttaaagaatttgctaaaaaaaaaggattagttgcaaaaaacttcattgaggatgccttttctaatgaaaccaaagataaagttattaagtatataaaggatgaatttggtaaaatagatttatttgtttatagtttagctgcgcctaggagaaaggactataaaactggaaatgtttatacttcaagaataaaaacaattttaggagattttgagggaccgactattgatgttgaaagagacgagattactttaaaaaaggttagtagtgctagcattgaagaaattgaagaaactagaaaggtaatgggtggagaggattggcaagagtggtgtgaagagctgctttatgaagattgtttttcggataaagcaactaccatagcatactcgtatataggatccccaagaacctacaagatatatagagaaggtactataggaatagctaaaaaggatcttgaagataaggctaagcttataaatgaaaaacttaacagagttataggtggtagagcctttgtgtctgtgaataaagcattagttacaaaagcaagtgcatatattccaacttttcctctttatgcagctattttatataaggtcatgaaagaaaaaaatattcatgaaaattgtattatgcaaattgagagaatgttttctgaaaaaatatattcaaatgaaaaaatacaatttgatgacaagggaagattaaggatggacgatttagagcttagaaaagacgttcaagacgaagttgatagaatatggagtaatattactcctgaaaattttaaggaattatctgattataagggatacaaaaaagaattcatgaacttaaacggttttgatctagatggggttgattatagtaaagacctggatatagaattattaagaaaattagaaccttaa

SEQ ID NO: 10 - protein, 398 aa, Clostridium acetobutylicum
MIVKAKFVKGFIRDVHPYGCRREVLNQIDYCKKAIGFRGPKKVLIVGASSGFGLATRISVAFGGPEAHTIGVSYETGATDRRIGTAGWYNNIFFKEFAKKKGLVAKNFIEDAFSNETKDKVIKYIKDEFGKIDLFVYSLAAPRRKDYKTGNVYTSRIKTILGDFEGPTIDVERDEITLKKVSSASIEEIEETRKVMGGEDWQEWCEELLYEDCFSDKATTIAYSYIGSPRTYKIYREGTIGIAKKDLEDKAKLINEKLNRVIGGRAFVSVNKALVTKASAYIPTFPLYAAILYKVMKEKNIHENCIMQIERMFSEKIYSNEKIQFDDKGRLRMDDLELRKDVQDEVDRIWSNITPENFKELSDYKGYKKEFMNLNGFDLDGVDYSKDLDIELLRKLEP

SEQ ID NO: 11 - DNA, 1407 bp, Clostridium beijerinckii
atgaataaagacacactaatacctacaactaaagatttaaaagtaaaaacaaatggtgaaaacattaatttaaagaactacaaggataattcttcatgtttcggagtattcgaaaatgttgaaaatgctataagcagcgctgtacacgcacaaaagatattatcccttcattatacaaaagagcaaagagaaaaaatcataactgagataagaaaggccgcattacaaaataaagaggtcttggctacaatgattctagaagaaacacatatgggaagatatgaggataaaatattaaaacatgaattggtagctaaatatactcctggtacagaagatttaactactactgcttggtcaggtgataatggtcttacagttgtagaaatgtctccatatggtgttataggtgcaataactccttctacgaatccaactgaaactgtaatatgtaatagcataggcatgatagctgctggaaatgctgtagtatttaacggacacccatgcgctaaaaaatgtgttgcctttgctgttgaaatgataaataaggcaattatttcatgtggcggtcctgaaaatctagtaacaactataaaaaatccaactatggagtctctagatgcaattattaagcatccttcaataaaacttctttgcggaactgggggtccaggaatggtaaaaaccctcttaaattctggtaagaaagctataggtgctggtgctggaaatccaccagttattgtagatgatactgctgatatagaaaaggctggtaggagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattcttagatgaaatagatgttgagtctccttcaaatgttaaatgcataatctgcgaagtaaatgcaaatcatccatttgttatgacagaactcatgatgccaatattgccaattgtaagagttaaagatatagatgaagctattaaatatgcaaagatagcagaacaaaatagaaaacatagtgcctatatttattctaaaaatatagacaacctaaatagatttgaaagagaaatagatactactatttttgtaaagaatgctaaatcttttgctggtgttggttatgaagcagaaggatttacaactttcactattgctggatctactggtgagggaataacctctgcaaggaattttacaagacaaagaagatgtgtacttgccggctaa

SEQ ID NO: 12 - protein, 468 aa, Clostridium beijerinckii
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 13 - DNA, 1215 bp, Clostridium acetobutylicum
atggttgatttcgaatattcaataccaactagaatttttttcggtaaagataagataaatgtacttggaagagagcttaaaaaatatggttctaaagtgcttatagtttatggtggaggaagtataaagagaaatggaatatatgataaagctgtaagtatacttgaaaaaaacagtattaaattttatgaacttgcaggagtagagccaaatccaagagtaactacagttgaaaaaggagttaaaatatgtagagaaaatggagttgaagtagtactagctataggtggaggaagtgcaatagattgcgcaaaggttatagcagcagcatgtgaatatgatggaaatccatgggatattgtgttagatggctcaaaaataaaaagggtgcttcctatagctagtatattaaccattgctgcaacaggatcagaaatggatacgtgggcagtaataaataatatggatacaaacgaaaaactaattgcggcacatccagatatggctcctaagttttctatattagatccaacgtatacgtataccgtacctaccaatcaaacagcagcaggaacagctgatattatgagtcatatatttgaggtgtattttagtaatacaaaaacagcatatttgcaggatagaatggcagaagcgttattaagaacttgtattaaatatggaggaatagctcttgagaagccggatgattatgaggcaagagccaatctaatgtgggcttcaagtcttgcgataaatggacttttaacatatggtaaagacactaattggagtgtacacttaatggaacatgaattaagtgcttattacgacataacacacggcgtagggcttgcaattttaacacctaattggatggagtatattttaaataatgatacagtgtacaagtttgttgaatatggtgtaaatgtttggggaatagacaaagaaaaaaatcactatgacatagcacatcaagcaatacaaaaaacaagagattactttgtaaatgtactaggtttaccatctagactgagagatgttggaattgaagaagaaaaattggacataatggcaaaggaatcagtaaagcttacaggaggaaccataggaaacctaagaccagtaaacgcctccgaagtcctacaaatattcaaaaaatctgtgtaaaacgcctccgaagtcctacaaatattcaaaaaatctgtgtaa

SEQ ID NO: 14 - protein, 390 aa, Clostridium acetobutylicum
MVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 15 - DNA, 1170 bp, Clostridium acetobutylicum
atgctaagttttgattattcaataccaactaaagttttttttggaaaaggaaaaatagacgtaattggagaagaaattaagaaatatggctcaagagtgcttatagtttatggcggaggaagtataaaaaggaacggtatatatgatagagcaacagctatattaaaagaaaacaatatagctttctatgaactttcaggagtagagccaaatcctaggataacaacagtaaaaaaaggcatagaaatatgtagagaaaataatgtggatttagtattagcaatagggggaggaagtgcaatagactgttctaaggtaattgcagctggagtttattatgatggcgatacatgggacatggttaaagatccatctaaaataactaaagttcttccaattgcaagtatacttactctttcagcaacagggtctgaaatggatcaaattgcagtaatttcaaatatggagactaatgaaaagcttggagtaggacatgatgatatgagacctaaattttcagtgttagatcctacatatacttttacagtacctaaaaatcaaacagcagcgggaacagctgacattatgagtcacacctttgaatcttactttagtggtgttgaaggtgcttatgtgcaggacggtatagcagaagcaatcttaagaacatgtataaagtatggaaaaatagcaatggagaagactgatgattacgaggctagagctaatttgatgtgggcttcaagtttagctataaatggtctattatcacttggtaaggatagaaaatggagttgtcatcctatggaacacgagttaagtgcatattatgatataacacatggtgtaggacttgcaattttaacacctaattggatggaatatattctaaatgacgatacacttcataaatttgtttcttatggaataaatgtttggggaatagacaagaacaaagataactatgaaatagcacgagaggctattaaaaatacgagagaatactttaattcattgggtattccttcaaagcttagagaagttggaataggaaaagataaactagaactaatggcaaagcaagctgttagaaattctggaggaacaataggaagtttaagaccaataaatgcagaggatgttcttgagatatttaaaaaatcttattaa

SEQ ID NO: 16 - protein, 389 aa, Clostridium acetobutylicum
MLSFDYSIPTKVFFGKGKIDVIGEEIKKYGSRVLIVYGGGSIKRNGIYDRATAILKENNIAFYELSGVEPNPRITTVKKGIEICRENNVDLVLAIGGGSAIDCSKVIAAGVYYDGDTWDMVKDPSKITKVLPIASILTLSATGSEMDQIAVISNMETNEKLGVGHDDMRPKFSVLDPTYTFTVPKNQTAAGTADIMSHTFESYFSGVEGAYVQDGIAEAILRTCIKYGKIAMEKTDDYEARANLMWASSLAINGLLSLGKDRKWSCHPMEHELSAYYDITHGVGLAILTPNWMEYILNDDTLHKFVSYGINVWGIDKNKDNYEIAREAIKNTREYFNSLGIPSKLREVGIGKDKLELMAKQAVRNSGGTIGSLRPINAEDVLEIFKKSY

SEQ ID NO: 17 - DNA, 780 bp, Klebsiella pneumoniae
atgaatcattctgctgaatgcacctgcgaagagagtctatgcgaaaccctgcgggcgttttccgcgcagcatcccgagagcgtgctctatcagacatcgctcatgagcgccctgctgagcggggtttacgaaggcagcaccaccatcgcggacctgctgaaacacggcgatttcggcctcggcacctttaatgagctggacggggagctgatcgccttcagcagtcaggtctatcagctgcgcgccgacggcagcgcgcgcaaagcccagccggagcagaaaacgccgttcgcggtgatgacctggttccagccgcagtaccggaaaacctttgaccatccggtgagccgccagcagctgcacgaggtgatcgaccagcaaatcccctctgacaacctgttctgcgccctgcgcatcgacggccatttccgccatgcccatacccgcaccgtgccgcgccagacgccgccgtaccgggcgatgaccgacgtcctcgacgatcagccggtgttccgctttaaccagcgcgaaggggtgctggtcggcttccggaccccgcagcatatgcaggggatcaacgtcgccgggtatcacgagcactttattaccgatgaccgcaaaggcggcggtcacctgctggattaccagctcgaccatggggtgctgaccttcggcgaaattcacaagctgatgatcgacctgcccgccgacagcgcgttcctgcaggctaatctgcatcccgataatctcgatgccgccatccgttccgtagaaagttaa

SEQ ID NO: 18 - protein, 259 aa, Klebsiella pneumoniae
MNHSAECTCEESLCETLRAFSAQHPESVLYQTSLMSALLSGVYEGSTTIADLLKHGDFGLGTFNELDGELIAFSSQVYQLRADGSARKAQPEQKTPFAVMTWFQPQYRKTFDHPVSRQQLHEVIDQQIPSDNLFCALRIDGHFRHAHTRTVPRQTPPYRAMTDVLDDQPVFRFNQREGVLVGFRTPQHMQGINVAGYHEHFITDDRKGGGHLLDYQLDHGVLTFGEIHKLMIDLPADSAFLQANLHPDNLDAAIRSVES

SEQ ID NO: 19 - DNA, 1680 bp, Klebsiella pneumoniae
atggacaaacagtatccggtacgccagtgggcgcacggcgccgatctcgtcgtcagtcagctggaagctcagggagtacgccaggtgttcggcatccccggcgccaaaattgacaaggtcttcgactcactgctggattcctcgattcgcattattccggtacgccacgaagccaacgccgcgtttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccgtcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctgaaccagtttgccctgcatccgctgcgcatcgttcgcgccatgcaggacatcgtcaacagcgacgtcacgttgaccgtggacatgggcagcttccatatctggattgcccgctacctgtacagcttccgcgcccgtcaggtgatgatctccaacggccagcagaccatgggcgtcgccctgccctgggctatcggcgcctggctggtcaatcctgagcgaaaagtggtctccgtctccggcgacggcggcttcctgcagtcgagcatggagctggagaccgccgtccgcctgaaagccaacgtactgcacctgatctgggtcgataacggctacaacatggtggccattcaggaagagaaaaaataccagcgcctgtccggcgtcgagttcgggccgatggattttaaagcctatgccgaatccttcggcgcgaaagggtttgccgtggaaagcgccgaggcgctggagccgaccctgcacgcggcgatggacgtcgacggcccggcggtggtggccattccggtggattatcgcgataacccgctgctgatgggccagctgcatctgagtcagattctgtaa

SEQ ID NO: 20 - protein, 559 aa, Klebsiella pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAVEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYSFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLHAAMDVDGPAVVAIPVDYRDNPLLMGQLHLSQIL

SEQ ID NO: 21 - DNA, 771 bp, Klebsiella pneumoniae
atgaaaaaagtcgcacttgttaccggcgccggccaggggattggtaaagctatcgcccttcgtctggtgaaggatggatttgccgtggccattgccgattataacgacgccaccgccaaagcggtcgcctcggaaatcaaccaggccggcggacacgccgtggcggtgaaagtggatgtctccgaccgcgatcaggtatttgccgccgttgaacaggcgcgcaaaacgctgggcggcttcgacgtcatcgtcaataacgccggtgtggcaccgtctacgccgatcgagtccattaccccggagattgtcgacaaagtctacaacatcaacgtcaaaggggtgatctggggtattcaggcggcggtcgaggcctttaagaaagaggggcacggcgggaaaatcatcaacgcctgttcccaggccggccacgtcggcaacccggagctggcggtgtatagctccagtaaattcgcggtacgcggcttaacccagaccgccgctcgcgacctcgcgccgctgggcatcacggtcaacggctactgcccggggattgtcaaaacgccaatgtgggccgaaattgaccgccaggtgtccgaagccgccggtaaaccgctgggctacggtaccgccgagttcgccaaacgcatcactctcggtcgtctgtccgagccggaagatgtcgccgcctgcgtctcctatcttgccagcccggattctgattacatgaccggtcagtcgttgctgatcgacggcgggatggtatttaactaa

SEQ ID NO: 22 - protein, 256 aa, Klebsiella pneumoniae
MKKVALVTGAGQGIGKAIALRLVKDGFAVAIADYNDATAKAVASEINQAGGHAVAVKVDVSDRDQVFAAVEQARKTLGGFDVIVNNAGVAPSTPIESITPEIVDKVYNINVKGVIWGIQAAVEAFKKEGHGGKIINACSQAGHVGNPELAVYSSSKFAVRGLTQTAARDLAPLGITVNGYCPGIVKTPMWAEIDRQVSEAAGKPLGYGTAEFAKRITLGRLSEPEDVAACVSYLASPDSDYMTGQSLLIDGGMVFN

SEQ ID NO: 23 - DNA, 1665 bp, Klebsiella oxytoca
atgagatcgaaaagatttgaagcactggcgaaacgccctgtgaatcaggacggcttcgttaaggagtggatcgaagaaggctttatcgcgatggaaagcccgaacgacccaaaaccgtcgattaaaatcgttaacggcgcggtgaccgagctggacgggaaaccggtaagcgattttgacctgatcgaccactttatcgcccgctacggtatcaacctgaaccgcgccgaagaagtgatggcgatggattcggtcaagctggccaacatgctgtgcgatccgaacgttaaacgcagcgaaatcgtcccgctgaccaccgcgatgacgccggcgaaaattgtcgaagtggtttcgcatatgaacgtcgtcgagatgatgatggcgatgcagaaaatgcgcgcccgccgcaccccgtcccagcaggcgcacgtcaccaacgtcaaagataacccggtacagattgccgccgacgccgccgaaggggcatggcgcggatttgacgaacaggaaaccaccgttgcggtagcgcgctatgcgccgttcaacgccatcgcgctgctggtgggctcgcaggtaggccgtccgggcgtgctgacgcagtgctcgctggaagaagccaccgagctgaagctcggcatgctgggccacacctgctacgccgaaaccatctccgtctacggcaccgagccggtctttaccgacggcgacgacacgccgtggtcgaagggcttcctcgcctcgtcctacgcctctcgcgggctgaaaatgcgctttacctccggctccggctcggaagtgcagatgggctacgccgaaggcaaatccatgctttatctggaagcgcgctgcatctacatcaccaaagccgcgggcgtacagggtctgcaaaacggttccgtaagctgcatcggcgtgccgtctgcggtgccttccggcattcgcgcggtgctggcggaaaacctgatctgttcgtcgctggatctggagtgcgcctccagcaacgaccagaccttcacccactccgatatgcgtcgtaccgcgcgcctgctgatgcagttcctgccgggcaccgactttatctcctccggttattccgcggtgccgaactacgacaacatgttcgccggctccaacgaagatgccgaagactttgacgactacaacgtcatccagcgcgacctgaaggtggacggcggtttgcgtccggttcgcgaagaggacgtcatcgccatccgtaacaaagccgcccgcgcgctgcaggccgtgtttgccggaatggggctgccgccgattaccgatgaagaagttgaagccgcgacctacgcccacggttcgaaagatatgccggagcgcaacatcgtcgaagacatcaagttcgcccaggaaatcatcaataaaaaccgcaacggtctggaagtggtgaaagcgctggcgcagggcggattcaccgacgtggcccaggacatgctcaacatccagaaagctaagctgaccggggactacctgcatacctccgcgattatcgtcggcgacgggcaggtgctgtcagccgtcaacgacgtcaacgactatgccggtccggcaacgggctatcgcctgcagggcgaacgctgggaagagattaaaaacatccctggcgctcttgatcccaacgagattgattaa

SEQ ID NO: 24 - protein, 554 aa, Klebsiella oxytoca
MRSKRFEALAKRPVNQDGFVKEWIEEGFIAMESPNDPKPSIKIVNGAVTELDGKPVSDFDLIDHFIARYGINLNRAEEVMAMDSVKLANMLCDPNVKRSEIVPLTTAMTPAKIVEVVSHMNVVEMMMAMQKMRARRTPSQQAHVTNVKDNPVQIAADAAEGAWRGFDEQETTVAVARYAPFNAIALLVGSQVGRPGVLTQCSLEEATELKLGMLGHTCYAETISVYGTEPVFTDGDDTPWSKGFLASSYASRGLKMRFTSGSGSEVQMGYAEGKSMLYLEARCIYITKAAGVQGLQNGSVSCIGVPSAVPSGIRAVLAENLICSSLDLECASSNDQTFTHSDMRRTARLLMQFLPGTDFISSGYSAVPNYDNMFAGSNEDAEDFDDYNVIQRDLKVDGGLRPVREEDVIAIRNKAARALQAVFAGMGLPPITDEEVEAATYAHGSKDMPERNIVEDIKFAQEIINKNRNGLEVVKALAQGGFTDVAQDMLNIQKAKLTGDYLHTSAIIVGDGQVLSAVNDVNDYAGPATGYRLQGERWEEIKNIPGALDPNEID

SEQ ID NO: 25 - DNA, 675 bp, Klebsiella oxytoca
atggaaattaatgaaaaattgctgcgccagataattgaagacgtgctcagcgagatgaagggcagcgataaaccggtctcgtttaatgcgccggcggcctccgcggcgccccaggccacgccgcccgccggcgacggcttcctgacggaagtgggcgaagcgcgtcagggaacccagcaggacgaagtgattatcgccgtcggcccggctttcggcctggcgcagaccgtcaatatcgtcggcatcccgcataagagcattttgcgcgaagtcattgccggtattgaagaagaaggcattaaggcgcgcgtgattcgctgctttaaatcctccgacgtggccttcgtcgccgttgaaggtaatcgcctgagcggctccggcatctctatcggcatccagtcgaaaggcaccacggtgatccaccagcaggggctgccgccgctctctaacctggagctgttcccgcaggcgccgctgctgaccctggaaacctatcgccagatcggcaaaaacgccgcccgctatgcgaaacgcgaatcgccgcagccggtcccgacgctgaatgaccagatggcgcggccgaagtaccaggcgaaatcggccattttgcacattaaagagaccaagtacgtggtgacgggcaaaaacccgcaggaactgcgcgtggcgctttga

SEQ ID NO: 26 - protein, 224 aa, Klebsiella oxytoca
MEINEKLLRQIIEDVLSEMKGSDKPVSFNAPAASAAPQATPPAGDGFLTEVGEARQGTQQDEVIIAVGPAFGLAQTVNIVGIPHKSILREVIAGIEEEGIKARVIRCFKSSDVAFVAVEGNRLSGSGISIGIQSKGTTVIHQQGLPPLSNLELFPQAPLLTLETYRQIGKNAARYAKRESPQPVPTLNDQMARPKYQAKSAILHIKETKYVVTGKNPQELRVAL

SEQ ID NO: 27 - DNA, 522 bp, Klebsiella oxytoca
atgaataccgacgcaattgaatcgatggtacgcgacgtattgagccgcatgaacagcctgcagggcgaggcgcctgcggcggctccggcggctggcggcgcgtcccgtagcgccagggtcagcgactacccgctggcgaacaagcacccggaatgggtgaaaaccgccaccaataaaacgctggacgactttacgctggaaaacgtgctgagcaataaagtcaccgcccaggatatgcgtattaccccggaaaccctgcgcttacaggcttctattgccaaagacgcgggccgcgaccggctggcgatgaacttcgagcgcgccgccgagctgaccgcggtaccggacgatcgcattcttgaaatctacaacgccctccgcccctatcgctcgacgaaagaggagctgctggcgatcgccgacgatctcgaaagccgctatcaggcgaagatttgcgccgctttcgttcgcgaagcggccacgctgtacgtcgagcgtaaaaaactcaaaggcgacgattaa

SEQ ID NO: 28 - protein, 173 aa, Klebsiella oxytoca
MNTDAIESMVRDVLSRMNSLQGEAPAAAPAAGGASRSARVSDYPLANKHPEWVKTATNKTLDDFTLENVLSNKVTAQDMRITPETLRLQASIAKDAGRDRLAMNFERAAELTAVPDDRILEIYNALRPYRSTKEELLAIADDLESRYQAKICAAFVREAATLYVERKKLKGDD

SEQ ID NO: 29 - DNA, 1041 bp, Rhodococcus ruber
atgaaagccctccagtacaccgagatcggctccgagccggtcgtcgtcgacgtccccaccccggcgcccgggccgggtgagatcctgctgaaggtcaccgcggccggcttgtgccactcggacatcttcgtgatggacatgccggcagagcagtacatctacggtcttcccctcaccctcggccacgagggcgtcggcaccgtcgccgaactcggcgccggcgtcaccggattcgagacgggggacgccgtcgccgtgtacgggccgtgggggtgcggtgcgtgccacgcgtgcgcgcgcggccgggagaactactgcacccgcgccgccgagctgggcatcaccccgcccggtctcggctcgcccgggtcgatggccgagtacatgatcgtcgactcggcgcgccacctcgtcccgatcggggacctcgaccccgtcgcggcggttccgctcaccgacgcgggcctgacgccgtaccacgcgatctcgcgggtcctgcccctgctgggacccggctcgaccgcggtcgtcatcggggtcggcggactcgggcacgtcggcatccagatcctgcgcgccgtcagcgcggcccgcgtgatcgccgtcgatctcgacgacgaccgactcgcgctcgcccgcgaggtcggcgccgacgcggcggtgaagtcgggcgccggggcggcggacgcgatccgggagctgaccggcggtgagggcgcgacggcggtgttcgacttcgtcggcgcccagtcgacgatcgacacggcgcagcaggtggtcgcgatcgacgggcacatctcggtggtcggcatccatgccggcgcccacgccaaggtcggcttcttcatgatcccgttcggcgcgtccgtcgtgacgccgtactggggcacgcggtccgagctgatggacgtcgtggacctggcccgtgccggccggctcgacatccacaccgagacgttcaccctcgacgagggacccacggcctaccggcggctacgcgagggcagcatccgcggccgcggggtggtcgtcccgggctga

SEQ ID NO: 30 - protein, 346 aa, Rhodococcus ruber
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDLDDDRLALAREVGADAAVKSGAGAADAIRELTGGEGATAVFDFVGAQSTIDTAQQVVAIDGHISVVGIHAGAHAKVGFFMIPFGASVVTPYWGTRSELMDVVDLARAGRLDIHTETFTLDEGPTAYRRLREGSIRGRGVVVPG

SEQ ID NO: 31 - DNA, 1476 bp, Escherichia coli
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 32 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 33 - DNA, 1851 bp, Escherichia coli
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 34 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 35 - DNA, 1662 bp, Lactococcus lactis
tctagacatatgtatactgtgggggattacctgctggatcgcctgcacgaactggggattgaagaaattttcggtgtgccaggcgattataacctgcagttcctggaccagattatctcgcacaaagatatgaagtgggtcggtaacgccaacgaactgaacgcgagctatatggcagatggttatgcccgtaccaaaaaagctgctgcgtttctgacgacctttggcgttggcgaactgagcgccgtcaacggactggcaggaagctacgccgagaacctgccagttgtcgaaattgttgggtcgcctacttctaaggttcagaatgaaggcaaatttgtgcaccatactctggctgatggggattttaaacattttatgaaaatgcatgaaccggttactgcggcccgcacgctgctgacagcagagaatgctacggttgagatcgaccgcgtcctgtctgcgctgctgaaagagcgcaagccggtatatatcaatctgcctgtcgatgttgccgcagcgaaagccgaaaagccgtcgctgccactgaaaaaagaaaacagcacctccaatacatcggaccaggaaattctgaataaaatccaggaatcactgaagaatgcgaagaaaccgatcgtcatcaccggacatgagatcatctcttttggcctggaaaaaacggtcacgcagttcatttctaagaccaaactgcctatcaccaccctgaacttcggcaaatctagcgtcgatgaagcgctgccgagttttctgggtatctataatggtaccctgtccgaaccgaacctgaaagaattcgtcgaaagcgcggactttatcctgatgctgggcgtgaaactgacggatagctccacaggcgcatttacccaccatctgaacgagaataaaatgatttccctgaatatcgacgaaggcaaaatctttaacgagcgcatccagaacttcgattttgaatctctgattagttcgctgctggatctgtccgaaattgagtataaaggtaaatatattgataaaaaacaggaggattttgtgccgtctaatgcgctgctgagtcaggatcgtctgtggcaagccgtagaaaacctgacacagtctaatgaaacgattgttgcggaacagggaacttcatttttcggcgcctcatccatttttctgaaatccaaaagccatttcattggccaaccgctgtgggggagtattggttatacctttccggcggcgctgggttcacagattgcagataaggaatcacgccatctgctgtttattggtgacggcagcctgcagctgactgtccaggaactggggctggcgatccgtgaaaaaatcaatccgatttgctttatcatcaataacgacggctacaccgtcgaacgcgaaattcatggaccgaatcaaagttacaatgacatcccgatgtggaactatagcaaactgccggaatcctttggcgcgacagaggatcgcgtggtgagtaaaattgtgcgtacggaaaacgaatttgtgtcggttatgaaagaagcgcaggctgacccgaatcgcatgtattggattgaactgatcctggcaaaagaaggcgcaccgaaagttctgaaaaagatggggaaactgtttgcggagcaaaataaaagctaaggatcc

SEQ ID NO: 36 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 37 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 38 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 39 - DNA, 1224 bp, Euglena gracilis
atggcgatgtttacgaccaccgcaaaagttattcagccgaaaattcgtggttttatttgcaccaccacccacccgattggttgcgaaaaacgtgttcaggaagaaatcgcatacgcacgcgcgcacccgccgaccagcccgggtccgaaacgtgtgctggttattggctgcagtacgggctatggcctgagcacccgtatcaccgcggcctttggttatcaggccgcaaccctgggcgtgtttctggcaggcccgccgaccaaaggccgtccggccgcggcgggttggtataatacggttgcgttcgaaaaagccgccctggaagcaggtctgtatgcacgttctctgaatggtgatgcgttcgattctaccacgaaagcccgcaccgtggaagcaattaaacgtgatctgggtaccgttgatctggtggtgtatagcattgcagcgccgaaacgtaccgatccggccaccggcgtgctgcataaagcgtgcctgaaaccgattggtgcaacctacaccaatcgtacggtgaacaccgataaagcagaagttaccgatgtgagtattgaaccggccagtccggaagaaatcgcagataccgtgaaagttatgggtggcgaagattgggaactgtggattcaggcactgagcgaagccggcgtgctggccgaaggcgcaaaaaccgttgcgtattcttatattggcccggaaatgacgtggccggtgtattggagtggcaccattggcgaagccaaaaaagatgttgaaaaagcggcgaaacgcatcacccagcagtacggctgtccggcgtatccggttgttgccaaagcgctggtgacccaggccagtagcgccattccggtggtgccgctgtatatttgcctgctgtatcgtgttatgaaagaaaaaggcacccatgaaggctgcattgaacagatggtgcgtctgctgacgacgaaactgtatccggaaaatggtgcgccgatcgtggatgaagcgggccgtgtgcgtgttgatgattgggaaatggcagaagatgttcagcaggcagttaaagatctgtggagccaggtgagtacggccaatctgaaagatattagcgattttgcaggttatcagaccgaatttctgcgtctgtttggctttggtattgatggtgtggattacgatcagccggttgatgttgaagcggatctgccgagcgccgcccagcagtaagtcgac

SEQ ID NO: 40 - protein, 405 aa, Euglena gracilis
MAMFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 41 - DNA, 1440 bp, Bacillus subtilis
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctat

SEQ ID NO: 42 - protein, 342 aa, Bacillus subtilis
MVKVYYNGDIKENVLAGKTVAVIGYGSQGHAHALNLKESGVDVIVGVRQGKSFTQAQEDGHKVFSVKEAAAQAEIIMVLLPDEQQQKVYEAEIKDELTAGKSLVFAHGFNVHFHQIVPPADVDVFLVAPKGPGHLVRRTYEQGAGVPALFAIYQDVTGEARDKALAYAKGIGGARAGVLETTFKEETETDLFGEQAVLCGGLSALVKAGFETLTEAGYQPELAYFECLHELKLIVDLMYEEGLAGMRYSISDTAQWGDFVSGPRVVDAKVKESMKEVLKDIQNGTFAKEWIVENQVNRPRFNAINASENEHQIEVVGRKLREMMPFVKQGKKKEAVVSVAQN

SEQ ID NO: 43 - DNA, 25 bp, Artificial sequence
  Synthetic construct, Nucleic Acid Sequence encoding PCR primer N378
atgagtgaaattgcagcaactatcg

SEQ ID NO: 44 - DNA, 32 bp, Artificial sequence
  Synthetic construct, Nucleic Acid Sequence encoding PCR primer N394
atgaattcatcataggaggaaaacgatgggac

SEQ ID NO: 45 - DNA, 48 bp, Artificial sequence
  Synthetic construct, Nucleic Acid Sequence encoding PCR primer N395
cgatagttgctgcaatttcactcatccttgaacctcctggatcaacgc

SEQ ID NO: 46 - DNA, 33 bp, Artificial sequence
  Synthetic construct, Nucleic Acid Sequence encoding PCR primer N396
ataagcttttaaaagacgcgaattagcacaacc

SEQ ID NO: 47 - DNA, 24 bp, Artificial sequence
  Synthetic construct, Nucleic Acid Sequence encoding PCR primer N374
atcataggaggaaaacgatgggac

SEQ ID NO: 48 - DNA, 23 bp, Artificial sequence
  Synthetic construct, Nucleic Acid Sequence encoding PCR primer N375
ccttgaacctcctggatcaacgc

SEQ ID NO: 49 - DNA, 25 bp, Artificial sequence
  Synthetic construct, Nucleic Acid Sequence encoding PCR primer N379
ctaatcagtcactccccagtgttct

SEQ ID NO: 50 - DNA, 25 bp, Artificial sequence
  Synthetic construct, Nucleic Acid Sequence encoding PCR primer N380
atggctggcatatttaaaatagtca

SEQ ID NO: 51 - DNA, 25 bp, Artificial sequence
  Synthetic construct, Nucleic Acid Sequence encoding PCR primer N381
ttaaaagacgcgaattagcacaacc

SEQ ID NO: 52 - DNA, 25 bp, Artificial sequence
  Synthetic construct, Nucleic Acid Sequence encoding PCR primer N435
gctctaaatcaggacacccgccgat

SEQ ID NO: 53 - DNA, 25 bp, Artificial sequence
  Synthetic construct, Nucleic Acid Sequence encoding PCR primer N436
cgatagttgctgcaatttcactcat

SEQ ID NO: 54 - DNA, 25 bp, Artificial sequence
  Synthetic construct, Nucleic Acid Sequence encoding PCR primer N437
tgacactcgccaatcctcagagtgc

SEQ ID NO: 55 - DNA, 23 bp, Artificial sequence
  Synthetic construct, Nucleic Acid Sequence encoding PCR primer N438
gcgttgatccaggaggttcaagg

SEQ ID NO: 56 - DNA, 26 bp, Artificial sequence
  Synthetic construct, Nucleic Acid Sequence encoding PCR primer N376
agcgaccaattatcattgcgttagat

SEQ ID NO: 57 - DNA, 27 bp, Artificial sequence
  Synthetic construct, Nucleic Acid Sequence encoding PCR primer N377
ttagttactccattctgtcataatatc

SEQ ID NO: 58 - DNA, 24 bp, Artificial sequence
  Synthetic construct, Nucleic Acid Sequence encoding PCR primer N452
aagcgaccaattatcattgcgtta

SEQ ID NO: 59 - DNA, 35 bp, Artificial sequence
  Synthetic construct, Nucleic Acid Sequence encoding PCR primer N453
ggatgcatttagttactccattctgtcataatatc

SEQ ID NO: 60 - DNA, 25 bp, Artificial sequence
  Synthetic construct, Nucleic Acid Sequence encoding PCR primer N450
gttccacagggtagccagcagcatc

SEQ ID NO: 61 - DNA, 48 bp, Artificial sequence
  Synthetic construct, Nucleic Acid Sequence encoding PCR primer N451
aacgcaatgataattggtcgcttactaaaaatgcccatattttttcct

SEQ ID NO: 62 - DNA, 36 bp, Artificial sequence
  Synthetic construct, Nucleic Acid Sequence encoding PCR primer oBP15
atatagatctaaaatggtgaacaagcgattgcacgc

SEQ ID NO: 63 - DNA, 37 bp, Artificial sequence
  Synthetic construct, Nucleic Acid Sequence encoding PCR primer oBP16
atatcccgggagttagtcgctcctttcacggcgtttg

SEQ ID NO: 64 - DNA, 40 bp, Artificial sequence
  Synthetic construct, Nucleic Acid Sequence encoding PCR primer oBP17e
tatacccgggtcgttggtgcaactgattcaaaatagaaag

SEQ ID NO: 65 - DNA, 34 bp, Artificial sequence
  Synthetic construct, Nucleic Acid Sequence encoding PCR primer oBP18
tataggtaccgtcatcgtaatcttgtcagcatcc

SEQ ID NO: 66 - DNA, 22 bp, Artificial sequence
  Synthetic construct, Nucleic Acid Sequence encoding PCR primer oBP42
ctgtttctcacgctttctatcg

SEQ ID NO: 67 - DNA, 22 bp, Artificial sequence
  Synthetic construct, Nucleic Acid Sequence encoding PCR primer oBP45
aatgattcttagtttagggaat

SEQ ID NO: 68 - DNA, 21 bp, Artificial sequence
  Synthetic construct, Nucleic Acid Sequence encoding PCR primer oBP52
atattcgtcttcgatcttatc

SEQ ID NO: 69 - DNA, 53 bp, Artificial sequence
  Synthetic construct, Nucleic Acid Sequence encoding fabZ1(S.D.) F(SpeI)
tagactagtcaggaggggttaaaatgagtgtgttagaagcaagtgaaattatg

SEQ ID NO: 70 - DNA, 40 bp, Artificial sequence
  Synthetic construct, Nucleic Acid Sequence encoding fabZ1-R(BglII/XmaI)
cgacccgggagatctctattttgaatcagttgcaccaacg

SEQ ID NO: 71 - DNA, 42 bp, Artificial sequence
  Synthetic construct, Nucleic Acid Sequence encoding ClpL-F
tgacgcgtacttaagtggcaatattaacgataagtagttggc

SEQ ID NO: 72 - DNA, 6876 bp, Artificial sequence
  Synthetic construct, Nucleic Acid Sequence encoding pFP996 PclpL
gatctgtttaaacgcggccgcgctcgagcccgggatcgatggtacctcgcgaaagcttggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattaacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgtaggcctgacgcgtacttaagtggcaatattaacgataagtagttggccttaacttgatactgggtgtgctttttgagtcgtagtctcttaaattaaaattggtctggaaagtaacttgttaattcaagttctttatcaagggctattaaatagggaaccttgtagggaaatcaattcattttatgaaattgatttctcttttgtttaaacaaaattattctaattatttctaattgattctataagcactttacttaacgatacataaaaatcgtgatacgattcagttgtagttttaaaggatattatggtcactaagttacacatataaatttttatgactagtcaggaggggttaaaatggttgattttgaatactcaattccgactcgtatcttctttggtaaggacaagatcaacgttttgggccgagaattgaaaaagtacggttcaaaagtgttaattgtttatggtggtggttcaatcaagcgcaatggtatctatgataaagcagtcagtatcttagaaaagaattcaatcaaattttacgaattggcgggtgtcgaaccgaatccgcgcgtgacgactgtggaaaaaggtgttaagatttgtcgcgaaaatggtgtcgaagttgtgttagctattggcggtggcagtgcaattgattgtgctaaggtcatcgccgctgcctgtgaatatgacggtaacccatgggatattgtcttggatggtagtaagattaaacgggtgttaccgattgcaagtattttaaccattgcggcgacgggttcagaaatggatacgtgggctgtcattaacaatatggatacgaatgaaaaattgattgcggctcatccagacatggccccaaaattctcaattttggacccaacctatacgtatactgttccaacgaaccaaacggcagctggtacggccgatatcatgagtcatatttttgaagtgtattttagtaacacgaaaaccgcatatttgcaagaccggatggcggaagcgttattgcgtacttgtattaagtatggtggcattgctttagaaaagccagatgattacgaagctcgtgccaatttaatgtgggccagttcattagccattaatggcttattgacttacggtaaggatactaattggtcagttcacttaatggaacacgaattaagtgcatactacgacattacgcatggtgttggtttagccattttaacgccaaattggatggaatacattttgaacaacgataccgtttacaagtttgtcgaatatggtgtgaacgtttggggtatcgataaggaaaagaaccactatgacattgcacatcaagcaattcagaaaactcgtgactattttgtcaatgttttaggcttaccaagtcgtttgcgggatgttggtattgaagaagaaaaattggatattatggctaaagaaagtgttaagttaaccggtggtactattggtaacttacgtccagtgaatgcaagtgaagtcttacaaatctttaagaaatcagtttaaca

SEQ ID NO: 73 - DNA, 6123 bp, Artificial sequence
  Synthetic construct, Nucleic Acid Sequence encoding pFP996 PclpL-fabZ1
ccgggatcgatggtacctcgcgaaagcttggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattaacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgtaggcctgacgcgtacttaagtggcaatattaacgataagtagttggccttaacttgatactgggtgtgctttttgagtcgcagtctcttaaattaaaattggtctggaaagtaacttgttaattcaagttctttatcaagggctattaaatagggaaccttgtagggaaatcaattcattttatgaaattgatttctcttttgtttaaacaaaattattctaattatttctaattgattctataagcactttacttaacgatacataaaaatcgtgatacgattcagttgtagttttaaaggatattatggtcactaagttacacatataaatttttatgactagtcaggaggggttaaaatgagtgtgttagaagcaagtgaaattatgcaattaatccccaaccggtacccaattttattcatggaccgggtggatgaattaaatccgggtgaatcgatcgtggtgacgaaaaatgtcacgattaatgagtcatttttccaagggcactttcccggtaacccggtcatgccgggcgtgttgattattgaagctttggcgcaagccgcgtcgattctgattttgaaatctgaaaagtttgctggtaagacggcttatcttggcgccattaaggatgccaagttccgcaaaattgtccgtcccggtgatgtcttgaagttgcatgtccaaatggtcaagcaacggtccaacatgggaacggtgagttgtcaggcgatggtcggtgacaaggcagcctgcacaactgatttaacctttatcgttggtgcaactgattcaaaatagagatctc

SEQ ID NO: 74 - DNA, 31 bp, Artificial sequence
  Synthetic construct, Nucleic Acid Sequence encoding PCR primer left-arm-up
attcagatctccagttagtaggagtgattag

SEQ ID NO: 75 - DNA, 31 bp, Artificial sequence
  Synthetic construct, Nucleic Acid Sequence encoding PCR primer left-arm-down
tttactcgagcaagcaatgatacaatctgtt

SEQ ID NO: 76 - DNA, 31 bp, Artificial sequence
  Synthetic construct, Nucleic Acid Sequence encoding PCR primer right-arm-up
tttacccgggcgtgaaaggagcgactaacta

SEQ ID NO: 77 - DNA, 31 bp, Artificial sequence
  Synthetic construct, Nucleic Acid Sequence encoding PCR primer right-arm-down
atcctgtacacgaccattcatctgaaaggcc

SEQ ID NO: 78 - DNA, 31 bp, Artificial sequence
  Synthetic construct, Nucleic Acid Sequence encoding PCR primer PclpL-up
cttgctcgagtaaattaaaattggtctggaa

SEQ ID NO: 79 - DNA, 31 bp, Artificial sequence
  Synthetic construct, Nucleic Acid Sequence encoding PCR primer PclpL-down
cacgcccgggtaaaaatttatatgtgtaact

SEQ ID NO: 80 - DNA, 21 bp, Artificial sequence
  Synthetic construct, Nucleic Acid Sequence encoding PCR primer PfabZ1chromosome-up
cgaccacgggtgcgttattta

SEQ ID NO: 81 - DNA, 21 bp, Artificial sequence
  Synthetic construct, Nucleic Acid Sequence encoding PCR primer PfabZ1chromosome-down
aagcacaaatgctttaatatc

SEQ ID NO: 82 - DNA, 347 bp, Artificial sequence
  Synthetic construct, Nucleic Acid Sequence encoding cydA promoter region
tcgcgggctaaacttgttaaatgaggtacttttttaataacggcatcagttgtttattgtatgaactaatttgttaactgattgagtacgatgattgatttttatgatgcgacaagctcagttattcgcgggagtcacgcgtgattttaccagcagatgccagattgcaaaatgtgaacttacaaacgaataagcgtactaatagtggccttgaaaattagcggctttgactattttgggaataaaagttgagattgattgaaatttcacttatcacttgctattatgaaaggtgaataaagtgtttccgctttcgtagggaataaattaataaaggggggaccatt

SEQ ID NO: 83 - DNA, 282 bp, Artificial sequence
  Synthetic construct, Nucleic Acid Sequence encoding atpB promoter region
gtaaatgagaagtaggccgtcattgcgcgtgccaagaatgaaaataaagtcaaaataatgaaaatccaacgatttgaaagcttaatgaaagcttgatattgttggatttttattgattgacgaaatgttgaaattattttcaattttttcgacggtggtggtattattacctttgtattttgattaggggtgtctctaatctaccatttcaggttacgataaaattgacgttgactagctcaaaggttaaggttatcgtagcaccgaaattaaaggaaagag

SEQ ID NO: 84 - DNA, 351 bp, Artificial sequence
  Synthetic construct, Nucleic Acid Sequence encoding agrB promoter region
cgcgtacttaagtagcttaaatcagtgttcataaataactttatgtaagaggagattgcttgatagatctccttttttcgttgattttcaccatttgatattaatctattgagtaatgtgaattagtgaaattaagtctagtttgacttgtaatttaaggaaaaattaagggtgagtatcgatgaaactgatttatattaacgatctttttacatgaaactttagtttcgtatgaataattaaactgatgtatctttaaatgtttatttctagtcttaaaacaatattgaataattaatctatttatgtattcttttcaattaattaatactgttttaaactgttgataga

SEQ ID NO: 85 - DNA, 29 bp, Artificial sequence
  Synthetic construct, Nucleic Acid Sequence encoding PCR primer LDH EcoRV F
gacgtcatgaccacccgccgatccctttt

SEQ ID NO: 86 - DNA, 30 bp, Artificial sequence
  Synthetic construct, Nucleic Acid Sequence encoding PCR primer LDH AatIIR
gatatccaacaccagcgaccgacgtattac

SEQ ID NO: 87 - DNA, 6509 bp, Artificial sequence
  Synthetic construct, Nucleic Acid Sequence encoding pFP988
tcgaggccccgcacatacgaaaagactggctgaaaacattgagcctttgatgactgatgatttggctgaagaagtggatcgattgtttgagaaaagaagaagaccataaaaataccttgtctgtcatcagacagggtattttttatgctgtccagactgtccgctgtgtaaaaaataggaataaaggggggttgttattattttactgatatgtaaaatataatttgtataaggaattgtgagcggataacaattcctacgaaaatgagagggagaggaaacatgattcaaaaacgaaagcggacagtttcgttcagacttgtgcttatgtgcacgctgttatttgtcagtttgccgattacaaaaacatcagccggatcccaccatcaccatcaccattaagaattcctagaaactccaagctatctttaaaaaatctagtaaatgcacgagcaacatcttttgttgctcagtgcattttttattttgtacactagatatttcttctccgcttaaatcatcaaagaaatctttatcacttgtaaccagtccgtccacatgtcgaattgcatctgaccgaattttacgtttccctgaataattctcatcaatcgtttcatcaattttatctttatactttatattttgtgcgttaatcaaatcataatttttatatgtttcctcatgatttatgtctttattattatagtttttattctctctttgattatgtctttgtatcccgtttgtattacttgatcctttaactctggcaaccctcaaaattgaatgagacatgctacacctccggataataaatatatataaacgtatatagatttcataaagtctaacacactagacttatttacttcgtaattaagtcgttaaaccgtgtgctctacgaccaaaactataaaacctttaagaactttctttttttacaagaaaaaagaaattagataaatctctcatatcttttattcaataatcgcatccgattgcagtataaatttaacgatcactcatcatgttcatatttatcagagctcgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttgcatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgctccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttcatgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcatgccacgcgtc

SEQ ID NO: 88 - DNA, 47 bp, Artificial sequence
  Synthetic construct, Nucleic Acid Sequence encoding PCR primer CmF
atttaaatctcgagtagaggatcccaacaaacgaaaattggataaag

SEQ ID NO: 89 - DNA, 29 bp, Artificial sequence
  Synthetic construct, Nucleic Acid Sequence encoding PCR primer CmR
acgcgttattataaaagccagtcattagg

SEQ ID NO: 90 - DNA, 58 bp, Artificial sequence
  Synthetic construct, Nucleic Acid Sequence encoding PCR primer P11 F-StuI
cctagcgctatagttgttgacagaatggacatactatgatatattgttgctatagcga

SEQ ID NO: 91 - DNA, 62 bp, Artificial sequence
  Synthetic construct, Nucleic Acid Sequence encoding PCR primer P11 R-SpeI
ctagtcgctatagcaacaatatatcatagtatgtccattctgtcaacaactatagcgctagg

SEQ ID NO: 92 - DNA, 38 bp, Artificial sequence
  Synthetic construct, Nucleic Acid Sequence encoding PldhL F-HindII
aagcttgtcgacaaaccaacattatgacgtgtctgggc

SEQ ID NO: 93 - DNA, 28 bp, Artificial sequence
  Synthetic construct, Nucleic Acid Sequence encoding PCR Primer PldhL R-BamHI
ggatcctcatcctctcgtagtgaaaatt

SEQ ID NO: 94 - protein, 147 aa, Lactobacillus plantarum strain WCFS1
MSVLEASEIMQLIPNRYPILFMDRVDELNPGESIVVTKNVTINESFFQGHFPGNPVMPGVLIIEALAQAASILILKSEKFAGKTAYLGAIKDAKFRKIVRPGDVLKLHVQMVKQRSNMGTVSCQAMVGDKAACTTDLTFIVGATDSK

SEQ ID NO: 95 - protein, 151 aa, Lactobacillus sakei subsp. sakei 23K
MTLLNTTEIMALIPNRYPIIYIDTVESLVPGEEVVAIKNVTINEQFMRGYRPDSPQMPNTLMIEALAQTASILILKSPEFFGKTAYLGAAKNVLFHQTVRPGDQIVFTVKLTKKKENMGVVQTNATVNGQMVCEAELTFVVAPRDDLLGKK

SEQ ID NO: 96 - protein, 147 aa, Lactobacillus plantarum strain JDM1; identical to SEQ ID NO: 94

SEQ ID NO: 97 - protein, 151 aa, Lactococcus lactis subsp. lactis IL1403
MTKKYAMTATEVMEVIPNRYPIMFIDYVDEISENKIVATKNVTINEEVFNGHFPGNPTFPGVLILESLAQAGSILILKKEEFQGKMAYIGGIDKAKFRQKVTPGDVMKLEFEITKFRGKVGTADAAAYVDGKKVTTCQFTFIVDEAAEQTN

SEQ ID NO: 98 - protein, 148 aa, Leuconostoc citreum KM20
MPVLTTTEIMDLIPNRYPILYMDYVEEMVPDESIVAVKNVTINEQFFQGHFPGNPVMPGVLIIESLAQAASILILSSPQFKGKTAYMTGIDDAKFKKMVVPGDVLKLHVTFGKLRANMGSVIVEAKVDGKTATSAELMFVVAPDETNE

SEQ ID NO: 99 - protein, 147 aa, Lactobacillus plantarum subsp. plantarum ATCC 14917; identical to SEQ ID NO: 94

SEQ ID NO: 100 - protein, 145 aa, Lactobacillus ultunensis DSM 16047
MNIKLFVNQNKAVDQVTINAAEIKQLTGNQSPLSLLDQVLEIDPGKSLVGLKNVSANESYFAGHFPGNPVMPGVLIIQTGIEAVQVLNGAKWHGKLSEIKKARFRKMVKPGDQLEIKISKKDSEIYEAKAMLNDDVACSVELLFS

SEQ ID NO: 101 - protein, 147 aa, Lactobacillus delbrueckii subsp. bulgaricus ATCC 11842
MTVLDSSQIQEIIPHRYPMLLIDKVIDLVPGESAVAIRNVTNNEAVFQGHFPGNPVLPGVLLVESLAQTGAVALLSADRFKGQTAYFGGIKNAKFRQIVKPGDQVKLEVTLEKVKGHIGLGQGIAWVDGKKACTAELTFMISGEKNV

SEQ ID NO: 102 - protein, 144 aa, Enterococcus faecalis V583
MKKVMTATEIMEMIPNRYPICYIDYVDEIIPNEKIIATKNVTINEEFFQGHFPGNPTMPGVLIIEALAQVGSILILKMDQFEGETAYIGGINKAKFRQKVVPGDVLKLHFEIVKLRDFVGIGKATAYVEDKKVCECELTFIVGR

SEQ ID NO: 103 - protein, 148 aa, Lactobacillus brevis ATCC 367
MSVLTAAEIMTLIPNRYPILFMDRVDELNPGESITCTKNVTINEEFFQGHFPGNPVMPGVLIIESLAQAASILILKSEQFQGETAYLGAIKQAKFRKVVRPGDVLSLYVEMVKQRSNMGTVKCTASVGEKVACSADLTFIVAAADDKI

SEQ ID NO: 104 - protein, 149 aa, Pediococcus pentosaceus ATCC 25745
MSILNTTEIMELIPNRYPILFMDYVDELEPGKSIVATKNVTINEEFFQGHFPGNPVMPGVLIIESLAQAASILILKSEEFAGKTAYLGAINGAKFRQIVRPGDVLKLHVEMIKKKRNMGVVETFAMVGDKKVCQAELTFIVGATDKKDK

SEQ ID NO: 105 - protein, 148 aa, Lactobacillus helveticus DPC 4571
MSVLDAAEIMDLIPNRYPILFMDKVDELNPGESIVCTKNVTINEEFFQGHFPGNPVMPGVLIIESLAQAASILILKTEKYQGKTAYLGAIDSAKFRKVVRPGDVLKLHVTMEKQRDNMGKVKCEAKVEDKVACSAELTFIVPDPKKKI

SEQ ID NO: 106 - protein, 148 aa, Lactobacillus salivarius UCC118
MAIMDAQEIMDMIPNRYPICYIDYVDELVPGEKIIATKNVTINESFFRGHFPGNPVMPGVLLIETLAQAASILILKSPEFVGKTAYLGSISKAKFRKVVRPGDVLKLNVEMKKKHENMGIVDTQVIVNGKKACTAELMFIVADRDKKL

SEQ ID NO: 107 - DNA, 444 bp, Lactobacillus plantarum WCFS1
atgagtgtgttagaagcaagtgaaattatgcaattaatccccaaccggtacccaattttattcatggaccgggtggatgaattaaatccgggtgaatcgatcgtggtgacgaaaaatgtcacgattaatgagtcatttttccaagggcactttcccggtaacccggtcatgccgggcgtgttgattattgaagctttggcgcaagccgcgtcgattctgattttgaaatctgaaaagtttgctggtaagacggcttatcttggcgccattaaggatgccaagttccgcaaaattgtccgtcccggtgatgtcttgaagttgcatgtccaaatggtcaagcaacggtccaacatgggaacggtgagttgtcaggcgatggtcggtgacaaggcagcctgcacaactgatttaacctttatcgttggtgcaactgattcaaaatag

SEQ ID NO: 108 - DNA, 456 bp, Lactobacillus sakei subsp. sakei 23K
atgacactcttaaatacaactgagattatggcgctaattccaaatcggtacccgattatttatatcgatactgttgagtcgttagtacctggtgaagaagtggtggcaatcaagaacgtcacgattaatgaacagttcatgcgtggctatcgtcccgattcaccacagatgccaaatacattaatgattgaagccttggcacagacagcttcaatattaattctaaaatcaccagaattctttgggaagacagcttacctaggcgctgctaaaaacgttttgttccaccaaacggttcggcccggtgatcaaatcgtcttcacggttaaattaactaagaaaaaagaaaatatgggagttgtccaaaccaatgcgactgttaatggtcaaatggtttgtgaagcggagctaacctttgttgtggccccgcgtgatgatctcctcggaaaaaagtag

SEQ ID NO: 109 - DNA, 444 bp, Lactobacillus plantarum JDM1
atgagtgtgttagaagcaagtgaaattatgcaattaatccccaaccggtacccaattttattcatggaccgggtggatgaattaaatccgggtgaatcgatcgtggtgacgaaaaatgtcacgatcaatgagtcatttttccaagggcactttcccggtaacccggtcatgccgggcgtgttgattattgaagctttggcgcaagccgcgtcgattctgattttgaaatctgaaaagtttgctggtaagacggcttatcttggcgccattaaggatgccaagttccgcaaaattgtccgtcccggtgatgtcttgaagttgcatgtccaaatggtcaagcaacggtccaacatgggaacggtgagttgtcaggcgatggtcggtgacaaggcagcctgcacaactgatttaacctttatcgttggtgcaactgattcaaaatag

SEQ ID NO: 110 - DNA, 456 bp, Lactococcus lactis subsp. lactis Il1403
ttagtttgtttgttcagctgcttcatcaacgatgaaagtgaactgacaagtggttacttttttaccatcaacataagctgcggcatcagcagttcctacttttccacggaattttgtaatttcaaattcaagtttcattacatcaccaggagtgactttttgacggaattttgctttatcaattccaccaatataagccatttttccttgaaattcttcttttttgagaatcaaaattgaaccagcttgagcgagtgattcaagaatcaaaacaccagggaaagttggattaccagggaaatgtccattaaaaacttcttcgttaatcgttacatttttcgttgcaacaattttattttcagaaatttcatcaacgtagtcaataaacatgataggatagcggttcggaataacttccatcacttctgtagcagtcatagcgtattttttagtcat

SEQ ID NO: 111 - DNA, 447 bp, Leuconostoc citreum KM20
atgccagttcttacaacaacagaaattatggatcttattcccaatcgttatcccattctctatatggattacgttgaggagatggtaccagacgaatcaattgtagcggttaaaaacgtcacaattaatgaacaatttttccaaggtcattttccaggcaatccagtaatgccaggtgttctaattattgaatcactcgcccaagcagcctcaatcttgattttgtcttcaccccaatttaaaggtaagacagcttatatgacaggtattgatgacgccaagttcaagaaaatggttgtacctggtgatgttttgaagttgcatgttacttttggtaagcttcgcgcaaatatggggagcgtgattgtggaagcaaaagttgacgggaagacagcaacatcggcagagctgatgttcgttgttgcaccagatgaaactaatgaatga

SEQ ID NO: 112 - DNA, 444 bp, Lactobacillus plantarum subsp. plantarum ATCC 14917
ctattttgaatcagttgcaccaacgataaaggttaaatcagttgtgcaggctgccttgtcaccgaccatcgcctgacaactcaccgttcccatgttggaccgttgcttgaccatttggacatgcaacttcaagacatcaccgggacggacaattttgcggaacttggcatccttaatggcgccaagataagccgtcttaccagcaaacttttcagatttcaaaatcagaatcgacgcggcttgcgccaaagcttcaataatcaacacgcccggcatgaccgggttaccgggaaagtgcccttggaaaaatgactcattaatcgtgacatttttcgtcaccacgatcgattcacccggatttaattcatccacccggtccatgaataaaattgggtaccggttggggattaattgcataatttcacttgcttctaacacactcat

SEQ ID NO: 113 - DNA, 438 bp, Lactobacillus ultunensis DSM 16047
ctaagaaaaaagcaactccacactgcaagctacatcgtcatttaacatagcttttgcttcataaatttcactatcttttttactaattttgatctccaattgatcaccaggcttaaccattttacgaaagcgtgccttcttaatttcactaagctttccatgccacttagctccattaagaacctgaactgcttcaataccagtttgaataatcaagacacctggcattactgggtttcctggaaaatgtccagcaaagtaactttcattggcactgacatttttcaaaccaactaatgatttacctggatcaatctctaaaacttgatcaagtaagcttagtggagattgattacccgttaactgcttaatttcagcagcatttattgtcacctgatccaccgccttattttgatttacaaataatttgatattcaa

SEQ ID NO: 114 - DNA, 444 bp, Lactobacillus delbrueckii subsp. bulgaricus ATCC 11842
atgactgtattggattccagccaaatacaagaaattatcccccaccgctatcccatgcttttgattgacaaggtcatcgacctggttcccggcgaaagcgccgtggccatccgcaacgtgaccaataatgaggcggttttccagggacatttcccgggaaatcctgtcttgcccggggtcttgctcgtggaatccctggcccaaaccggggccgtggccctgttaagcgccgaccgcttcaaggggcagacggcctattttggcggtatcaaaaacgctaagttccgccagatagttaagcccggcgaccaggtcaagctggaagtgactttggaaaaggtcaagggccatatcggcctggggcagggaattgcctgggtcgacgggaagaaggcctgcacggcggaattgaccttcatgatttcaggtgagaaaaatgtttga

SEQ ID NO: 115 - DNA, 435 bp, Enterococcus faecalis V583
atgaaaaaagtaatgactgcaacagaaattatggaaatgattcctaatcgctatccgatttgttatattgattatgtggatgaaattattccaaatgaaaagattattgcaacaaaaaatgtgacaattaacgaagaatttttccaaggacatttccctggaaatccaacaatgccaggcgttttgattattgaagcattggcacaagtaggttcgattttaatcttaaaaatggatcaatttgaaggtgaaacagcctatattggcggtatcaacaaagccaaattccgtcaaaaagtggtccctggtgatgtcttgaaattacattttgaaatcgtcaaattacgtgactttgtcggcatcggcaaagcgactgcttacgtggaagataaaaaggtctgcgaatgtgaattgacgtttattgtgggacgataa

SEQ ID NO: 116 - DNA, 447 bp, Lactobacillus brevis ATCC 367
ttaaattttatcgtcagcagctgctacgataaaggttaagtcagccgaacacgcgaccttctcgccgacgcttgcggtacacttaaccgttcccatattactccgttgcttgaccatttcaacatataatgacaaaacatcccctggacggacaacttttctgaacttagcctgtttaatggcgcccagataagccgtctctccttgaaattgttccgactttaaaatcaaaatagatgcggcttgggccagcgactcaatgatcaagacgcctggcataaccggattaccgggaaaatggccttgaaaaaattcttcgttgatcgtgacatttttcgtacacgtaatggattctcccggattcaattcgtccacccgatccatgaataaaatagggtaacgattgggaatcaacgtcataatttcggcagccgtcaaaacactcat

SEQ ID NO: 117 - DNA, 450 bp, Pediococcus pentosaceus ATCC 25745
ttgagtattttaaatacaacagagattatggaactaattcctaatcgttaccccattctattcatggactatgttgatgaattagaacctggaaaatcaatcgtggcgactaaaaacgtcacaatcaacgaagaatttttccaaggacattttcctggtaacccggttatgcctggagttttaatcattgaatctctagcacaagctgcatcaattctaattctaaaatcagaagaatttgcaggtaagacagcatatctaggtgccattaatggtgctaaatttagacagatcgtccgtcctggtgatgttttaaaacttcatgttgaaatgatcaagaaaaagagaaacatgggtgttgttgaaacatttgcaatggtcggtgataaaaaagtttgccaagcagaactaacattcattgttggagcaactgataagaaagataaatag

SEQ ID NO: 118 - DNA, 447 bp, Lactobacillus helveticus DPC 4571
ttagattttcttcttaggatcagggacgataaacgttaactctgcagaacaggcaaccttatcttcgacctttgcttcacacttgactttgcccatattgtcacgttgtttttccatggtgacgtgtagtttaaggacatcgcccggacgaacgacttttctgaatttggcactgtcaattgcccccagataagccgttttgccttgatatttctctgtctttaaaatcaaaattgaagcggcttgggcaagtgactcaatgatcaacacaccaggcatgactggattgccaggaaaatggccttggaaaaattcttcattaattgtcacgttcttggtacaaacaattgactcaccaggatttaattcgtcgaccttatccataaacaggattgggtaacggttcggaatcaaatccataatttcagctgcatctaatacactcat

SEQ ID NO: 119 - DNA, 447 bp, Lactobacillus salivarius UCC118
gtggcaattatggatgcacaggaaataatggatatgattcctaatcgctatccgatctgttacattgactatgttgatgagctagtacctggtgagaaaattatcgcaacaaaaaatgtaacaattaatgaatcttttttcagaggacattttccaggaaatcctgtaatgccgggagttttactaattgaaactttagctcaagctgcgtcaatacttattttgaaatctccagaatttgtagggaaaacagcttatttaggttctataagtaaagctaagtttagaaaagttgtcagaccgggcgatgttttaaaattaaatgtcgaaatgaaaaagaaacacgagaacatggggatagtagatactcaagttatcgtgaatggaaagaaagcttgtacagctgaattaatgtttatagttgcggatagagacaagaagttgtag

SEQ ID NO: 120 - protein, 172 aa, Escherichia coli BL21
MVDKRESYTKEDLLASGRGELFGAKGPQLPAPNMLMMDRVVKMTETGGNFDKGYVEAELDINPDLWFFGCHFIGDPVMPGCLGLDAMWQLVGFYLGWLGGEGKGRALGVGEVKFTGQVLPTAKKVTYRIHFKRIVNRRLIMGLADGEVLVDGRLIYTASDLKVGLFQDTSAF

SEQ ID NO: 121 - DNA, 519 bp, Escherichia coli BL21
tcagaaggcagacgtatcctggaacagaccgactttcaggtcgctggcggtatagatcagacgaccatcaaccagcacttcgccatccgccaggcccataatcagacgacggttaacaatgcgtttaaagtgaatacggtaggtcacttttttcgctgtcggcagtacctgaccagtgaatttcacttcgccaacgcccagcgcgcggcctttaccttcgccgcccagccagccgaggtagaaccctaccagctgccacattgcgtccaggcccaggcatcccggcataaccggatcgccaataaagtggcatccgaagaaccacagatccggattgatatccagttctgcttcaacataccctttgtcgaagttaccacccgtttcggtcattttgaccacacggtccatcatcagcatgttcggtgctggcaattgcgggcctttagcgccaaacagttcaccgcgaccagaggcaagaaggtcttcttttgtataggattcgcgtttatctaccat

SEQ ID NO: 122 - protein, 144 aa, Lactobacillus reuteri
MTNKTLDITEIQKILPHRYPMLLIDQVDELIPGKKAIARRNVTINEEVFNGHFPKNPVLPGALIVESLAQTGAVALLSQEEFQGKTAYFGGIRSAEFRKVVRPGDTLKLEVNLEKVHKNIGIGKGIATVDGKKACTAELTFMIG

SEQ ID NO: 123 - DNA, 435 bp, Lactobacillus reuteri
atgactaataaaactttagatataactgaaattcaaaaaatccttcctcatcgttacccaatgttactaattgaccaagttgatgaattaatccccggtaagaaggcaatcgcacggcgtaatgtcacgatcaatgaagaggtttttaatggccatttccccaaaaatccagttttaccaggagcattgattgttgaatcattggcgcaaacaggtgccgtcgctctcttatcacaagaagagttccaaggaaaaacagcctattttggtggaattcgatcagcagaatttcgtaaagtagttcgccctggtgacacattaaagttagaagtcaacctagaaaaagtgcataaaaacattggaattggtaaaggcattgcaacggtcgatggcaaaaaagcctgtacagccgaattaacttttatgattgggtag

SEQ ID NO: 124 - protein, 171 aa, Agrobacterium radiobacter K84
MTTRQSSFNYEEILSCGRGELFGPGNAQLPLPPMLMVHRITDISETGGAFDKGYIRAEYDVRPDDWYFPCHFAGNPIMPGCLGLDGMWQLTGFFLGWLGEPGRGMALSTGEVKFKGMVRPDTKLLEYGIDFKRVMRGRLVLGTADGYLKADGEVIYQASDLRVGLSKDKAA

SEQ ID NO: 125 - DNA, 516 bp, Agrobacterium radiobacter K84
atgacgacgagacaatccagcttcaactatgaggaaatcctgtcctgcggccgcggcgaattgttcggcccgggcaatgcgcagcttcccctaccaccgatgctgatggtccatcgcattacagatatttccgaaaccggtggtgctttcgacaagggttacattcgcgctgaatatgacgtgcgtcccgacgactggtacttcccctgccattttgccggcaatccgatcatgccgggctgcctcggccttgacggcatgtggcagctgaccggcttcttcctcggctggctcggcgagcctggccgcggcatggcgctgtcgaccggcgaagtgaagttcaagggcatggttcgtccagacacgaagctcctcgaatacggcatcgacttcaagcgcgtcatgcgcggccgtcttgttctcgggactgccgatggctacttgaaagccgacggcgaagttatttatcaggcgagcgacctgcgcgtcggcctgtcaaaggacaaggctgcctga

SEQ ID NO: 126 - protein, 263 aa, Streptococcus mutans UA159
MDFKEILYNVDNGVATLTLNRPEVSNGFNIPICEEILKAIDIAKKDDTVQILLINANGKVFSVGGDLVEMQRAVDADDVQSLVRIAELVNKISFALKRLPKPVVMSTDGAVAGAAANIAVAADFCIASDKTRFIQAFVNVGLAPDAGGLFLLTRAIGITRATQLAMTGEALNAEKALEYGIVYKVCEPEKLEKITDRVITRLKRGSVNSYKAIKEMVWQSSFAGWQEYEDLELELQKSLAFTNDFKEGVRAYTEKRRPKFTGK

SEQ ID NO: 127 - DNA, 789 bp, Streptococcus mutans UA159
atggattttaaggaaattctgtacaatgtggataatggtgtggcgactttaacgctgaatcgtccggaggtttctaatggatttaatatccctatttgtgaggaaattttgaaggccattgatattgctaaaaaggatgacacagtacaaattttactgattaatgccaatgggaaagtcttttcagttggtggcgatctggttgagatgcaaagagctgttgatgcagatgatgtacaatctcttgttcgcattgcagaacttgtcaataaaatttcttttgctttaaaacgtttacctaagccggttgtcatgagtacagatggtgcagttgcaggtgctgcagctaatatagcggtagctgcagacttttgtattgccagtgacaaaacacgctttattcaagcctttgtgaatgtcggtttggcccctgatgccggaggacttttcttattaacgagagccattggtattactcgtgcaacacaacttgccatgaccggtgaagctttaaatgcagagaaagctttggaatacggtattgtttacaaagtctgtgagccagagaaactagaaaaaataacagatcgtgtcattacacgtttgaaacgtggctcagttaattcttataaagccattaaagaaatggtttggcaaagttcatttgcaggttggcaggaatatgaggatctagaattagaattgcaaaagtcattagcatttacaaatgattttaaagagggagtgcgtgcttatacagagaaacgccgtcctaaatttacaggaaag

SEQ ID NO: 128 - protein, 147 aa, Lactobacillus plantarum PN0512; identical to SEQ ID NO: 94

SEQ ID NO: 129 - DNA, 444 bp, Lactobacillus plantarum PN0512; identical to SEQ ID NO: 107